(19) United States
(12) Patent Application Publication
(10) Pub. No.: US 2022/0140258 A1
(43) Pub. Date: May 5, 2022

(54) IRIDIUM COMPLEX, NITROGEN-CONTAINING TRIDENTATE CARBENE CHELATE, AND ORGANIC LIGHT-EMITTING DIODE

(71) Applicants: National Tsing Hua University, Hsinchu City (TW); City University of Hong Kong, Kowloon (HK)

(72) Inventors: Yun Chi, Hsinchu City (TW); Wun-Shan Tai, Hsinchu City (TW); Premkumar Gnanasekaran, Hsinchu City (TW); Ling-Yang Hsu, Hsinchu City (TW)

(73) Assignees: National Tsing Hua University, Hsinchu City (TW); City University of Hong Kong, Kowloon (HK)

(21) Appl. No.: 17/147,420

(22) Filed: Jan. 12, 2021

(30) Foreign Application Priority Data
Oct. 30, 2020 (TW) 109137772

(51) Int. Cl. H01L 51/00; C07F 15/00; C07D 471/04; C09K 11/06

(52) U.S. Cl. CPC H01L 51/0085; C07F 15/0033; C07D 471/04; C09K 11/06; H01L 51/5016; C09K 2211/1007; C09K 2211/1029; C09K 2211/1059; C09K 2211/185

(57) ABSTRACT

An iridium complex has the structure:

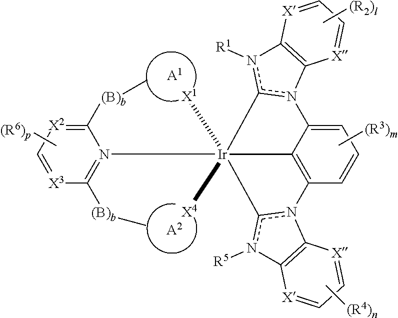

wherein X′ and X″ independently represent carbon or nitrogen; X¹, X², X³, and X⁴ independently represent carbon or nitrogen; R¹ and R⁵ independently represent substituted or unsubstituted C₁-C₁₂ alkyl; R², R³, and R⁴ independently represent hydrogen, C₁-C₁₂ alkyl, substituted or unsubstituted C₆-C₁₂ aryl, or —CₘF₂ₘ₊₁, m is from 1 to 3; each of m and n is from 1 to 3; A¹ and A² independently represent an unsaturated 5-membered or 6-membered ring; B is —O—, —NR— or —CR₂—, A² may join with —NR— or —CR₂— to form a C₉-C₁₄ N-heteroaromatic or aromatic ring; b is 0 or 1; R⁶ is hydrogen, fluorine, substituted or unsubstituted C₁-C₁₂ alkyl, substituted or unsubstituted C₁-C₆ alkoxyl, substituted or unsubstituted C₆-C₁₂ aryl, substituted or unsubstituted C₁-C₆ amino, or —CₓF₂ₓ₊₁, x is from 1 to 3; and p is from 1 to 3.

### CROSS-REFERENCE TO RELATED APPLICATION

This application claims the priority benefit of Taiwan application no. 109137772, filed on Oct. 30, 2020. The entirety of the above-mentioned patent application is hereby incorporated by reference herein and made a part of this specification.

### BACKGROUND OF THE INVENTION

### Field of the Invention

The invention relates to a metal complex and an application thereof, and particularly relates to an iridium complex suitable for an organic light-emitting diode (OLED) and to a nitrogen-containing tridentate carbene chelate suitable for forming the iridium complex.

### Description of Related Art

Organic light-emitting diode devices have attracted much attention in the display industry, especially in the flat-panel display industry, because the organic light-emitting diode devices may operate at a low driving voltage and may produce high light-emitting efficiency.

In order to develop full-color flat-panel displays, the main object of current OLED research is to develop and synthesize simple light-emitting materials with high efficiency. At present, it is known that the traditional tris-bidentate coordinated iridium complexes have suitable light emission characteristics, but their structural rigidity, both of the chemical and physical stability, and ease of synthesis thereof are still insufficient, in comparison to the bis-tridentate iridium complexes claimed in the present invention.

### SUMMARY OF THE INVENTION

The invention provides an iridium complex, a nitrogen-containing tridentate carbene chelate, and an application thereof. The iridium complex prepared by the invention has sufficiently high rigidity and stability and is easy to synthesize.

The invention provides an iridium complex having a structure represented by general formula (I):

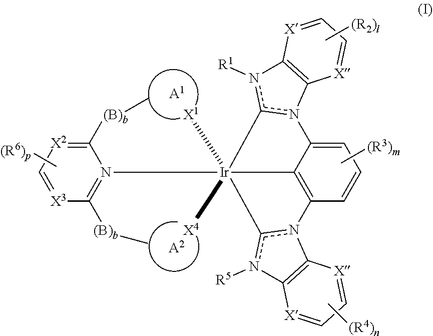

wherein

one of X′ and X″ is nitrogen and the other is carbon; X¹, X², X³, and X⁴ are each independently carbon or nitrogen; R¹ and R⁵ are each independently substituted or unsubstituted C₁-C₁₂ alkyl; R², R³, and R⁴ are each independently hydrogen, substituted or unsubstituted C₁-C₁₂ alkyl, substituted or unsubstituted C₆-C₁₂ aryl, or —CₘF₂ₘ₊₁, and m is an integer of 1 to 3; 1, m, and n are each independently an integer of 1 to 3; when 1 is equal to or greater than 2, two or more R²'s may be connected to each other to form a C₃-C₈ aromatic ring or a nitrogen-containing heteroaromatic ring; when m is equal to or greater than 2, two or more R³'s may be connected to each other to form a C₃-C₈ aromatic ring or a nitrogen-containing heteroaromatic ring; when n is equal to or greater than 2, two or more R⁴'s may be connected to each other to form a C₃-C₈ aromatic ring or a nitrogen-containing heteroaromatic ring; A¹ and A² are each independently an unsaturated 5-membered ring or an unsaturated 6-membered ring; B is —O—, —NR—, or —CR₂—, R is hydrogen or substituted or unsubstituted C₁-C₁₂ alkyl, and —NR— or —CR₂— may optionally be connected to A² to form a substituted or unsubstituted C₉-C₁₄ aromatic ring or a nitrogen-containing heteroaromatic ring; b is 0 or 1; R⁶ is hydrogen, fluorine, substituted or unsubstituted C₁-C₁₂ alkyl, substituted or unsubstituted C₁-C₆ alkoxy, substituted or unsubstituted C₆-C₁₂ aryl, substituted or unsubstituted C₁-C₆ amino, or —CₓF₂ₓ₊₁, and x is an integer of 1 to 3; p is an integer of 1 to 3; and when p is equal to or greater than 2, two or more RP's may be connected to each other to form a C₃-C₈ aromatic ring or a nitrogen-containing heteroaromatic ring.

The invention provides an organic light-emitting diode including two electrodes and a light-emitting layer disposed between the two electrodes, wherein the light-emitting layer includes the iridium complex above.

The invention provides an iridium complex having a structure represented by general formula (I-1) or (I-2):

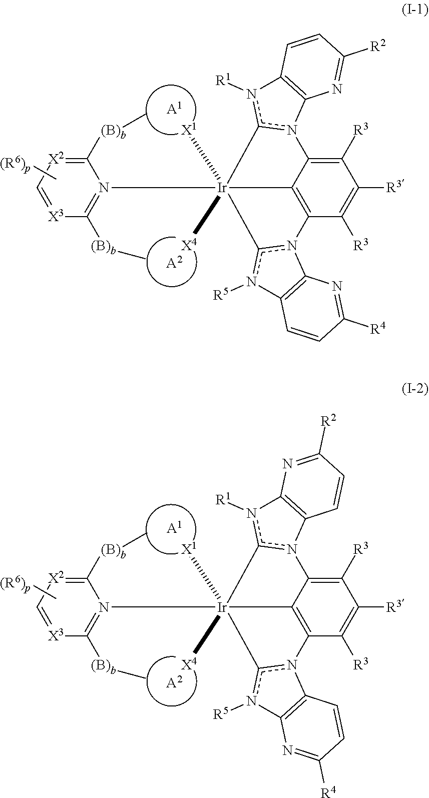

wherein X¹, X², X³, and X⁴ are each independently carbon or nitrogen; R¹ and R⁵ are each independently substituted or unsubstituted C₁-C₁₂ alkyl; R², R³, and R⁴ are each independently hydrogen, substituted or unsubstituted C₁-C₁₂ alkyl, or substituted or unsubstituted C₆-C₁₂ aryl; R^{3′} is an electron withdrawing group or an electron donating group, wherein the electron withdrawing group includes —CₘF₂ₘ₊₁, m is an integer of 0 to 3, and the electron donating group includes methyl, tert-butyl, or C₁-C₆ alkoxy; A¹ and A² are each independently an unsaturated 5-membered ring or an unsaturated 6-membered ring; B is —O—, —NR—, or —CR₂—, R is hydrogen or substituted or unsubstituted C₁-C₁₂ alkyl, and —NR— or —CR₂— may optionally be connected to A² to form a substituted or unsubstituted C₉-C₁₄ aromatic ring or a nitrogen-containing heteroaromatic ring; b is 0 or 1; R⁶ is hydrogen, fluorine, substituted or unsubstituted C₁-C₁₂ alkyl, substituted or unsubstituted C₁-C₆ alkoxy, substituted or unsubstituted C₆-C₁₂ aryl, substituted or unsubstituted C₁-C₆ amino, or —CₓF₂ₓ₊₁, and x is an integer of 1 to 3; p is an integer of 1 to 3; when p is equal to or greater than 2, two or more R⁶'s may be connected to each other to form a C₃-C₈ aromatic ring or a nitrogen-containing heteroaromatic ring; in general formula (I-1), at least one of R² and R³ is not hydrogen, and at least one of R³ and R⁴ is not hydrogen; and in general formula (I-2), R² and R⁴ are both not hydrogen, such that steric encumbrance is provided to protect the adjacent N atoms from being affected by the external environment.

The invention provides a nitrogen-containing tridentate carbene chelate having a structure represented by general formula (II):

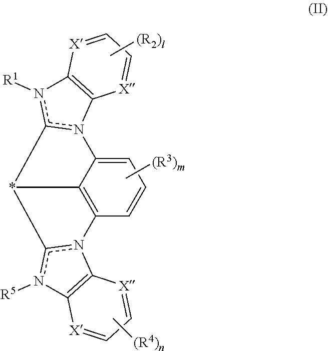

wherein
* represents a bonding site;
one of X′ and X″ is nitrogen, and the other is carbon; R¹ and R⁵ are each independently substituted or unsubstituted C₁-C₁₂ alkyl; R², R³, and R⁴ are each interpedently hydrogen, substituted or unsubstituted C₁-C₁₂ alkyl, substituted or unsubstituted C₆-C₁₂ aryl, or —CₘF₂ₘ₊₁, and m is an integer of 0 to 3; 1, m, and n are each independently an integer of 1 to 3; when 1 is equal to or greater than 2, two or more R²'s may be connected to each other to form a C₃-C₈ aromatic ring or a nitrogen-containing heteroaromatic ring; when m is equal to or greater than 2, two or more R³'s may be connected to each other to form a C₃-C₈ aromatic ring or a nitrogen-containing heteroaromatic ring; and when n is equal to or greater than 2, two or more R⁴'s may be connected to each other to form a C₃-C₈ aromatic ring or a nitrogen-containing heteroaromatic ring.

To sum up, the blue phosphorescent material provided by the invention may provide both the high efficiency and short photoluminescence lifetime, and through such material, a related OLED device may therefore exhibit favorable performance in efficiency.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 shows the single crystal X-ray diffraction structural diagram of complex (IA-40) synthesized in Example 1 of the invention.

FIG. 2 shows the respectively absorption spectra of conventional complexes (1) and (2) and complexes (IA-40) and (IA-80) of the invention dissolved in a dichloromethane solvent and the emission spectra thereof in the corresponding deoxygenated solution state.

FIG. 3 shows the respective emission spectra of conventional complexes (1) and (2) and complexes (IA-40) and (IA-80) of the invention dispersed in polymethyl methacrylate (PMMA) matrix.

FIG. 4 shows the respective absorption spectra of complexes (IA-40), (IA-42), (IA-48), (IA-52), (IA-60), (IA-64), (IA-74), (IA-80), and (IA-130) mentioned in the invention dissolved in a dichloromethane solvent and the emission spectra thereof in the corresponding deoxygenated solution state.

FIG. 5 shows the respective emission spectra of complexes (IA-40), (IA-42), (IA-48), (IA-52), (IA-60), (IA-64), (IA-74), and (IA-80) mentioned in the invention dispersed in polymethyl methacrylate matrix.

### DESCRIPTION OF THE EMBODIMENTS

The invention provides a blue phosphorescent material exhibiting both the short photoluminescence lifetime and high efficiency, and through such material, a related organic light-emitting diode (OLED) may exhibit favorable performance in efficiency.

To be more specific, in order to ultimately achieve a blue shifted emission, the energy level of a lowest unoccupied molecular orbital (LUMO) of a metal phosphorescent material that is shown to emit in the region of violet or near ultraviolet light is continuously reduced in the invention, giving the blue emission by an expected red-shifting effect may thus be achieved in this way.

For instance, an electron-withdrawing nitrogen atom is added to the benzo[d]imidazol-2-ylidene chelate of Ir(pmb)₃, in giving the imidazo[4,5-b]pyridin-2-ylidene chelate of Ir(pmp)₃ in the invention. Ir(pmb)₃ having either mer- or fac-arrangement of chelates and having a highest emission peak at 389 nm (mer-) and 395 nm (fac-) may be changed to Ir(pmp)₃, to which the emission peak is shifted to 418 nm (mer-) and 465 nm (fac-).

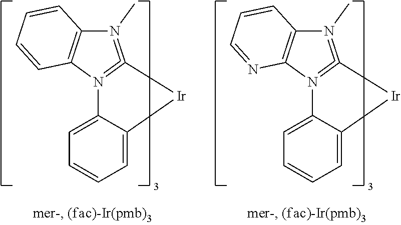

Through comparing emission wavelengths and photophysical characteristics of the two, it may be seen that the emission color is redshifted from purple to deep blue through the fine control of molecular structures as predicted by the fundamental molecular orbital theory. In this way, light-emitting efficiency of the complex is significantly improved, and that the complex may then be used as a blue emitting material featuring high efficiency. In these new carbene complexes, a metal-to-ligand charge transfer (MLCT) transition contribution may be increased due to the decrease in the relative emission energy, and the photoluminescence lifetime may thus be effectively shortened.

The following embodiments are used to further illustrate the invention. But the embodiments are provided only for description and are presented as examples and are not intended to limit the scope of the invention.

### [Structure of Iridium Complex]

The invention provides an iridium complex having a structure represented by general formula (I):

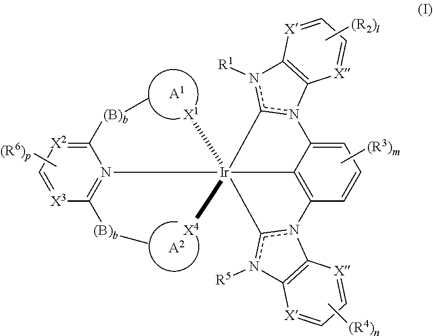

wherein
one of X′ and X″ is nitrogen and the other is carbon; X¹, X², X³, and X⁴ are each independently carbon or nitrogen; R¹ and R⁵ are each independently substituted or unsubstituted C₁-C₁₂ alkyl; R², R³, and R⁴ are each independently hydrogen, substituted or unsubstituted C₁-C₁₂ alkyl, substituted or unsubstituted C₆-C₁₂ aryl, or —CₘF₂ₘ₊₁, and m is an integer of 1 to 3; 1, m, and n are each independently an integer of 1 to 3; A¹ and A² are each independently an unsaturated 5-membered ring or an unsaturated 6-membered ring; B is —O—, —NR—, or —CR₂—, R is hydrogen or substituted or unsubstituted C₁-C₁₂ alkyl, and —NR— or —CR₂— may optionally be connected to A² to form a substituted or unsubstituted C₉-C₁₄ aromatic ring or a nitrogen-containing heteroaromatic ring; b is 0 or 1; R⁶ is hydrogen, fluorine, substituted or unsubstituted C₁-C₁₂ alkyl, substituted or unsubstituted C₁-C₆ alkoxy, substituted or unsubstituted C₆-C₁₂ aryl, substituted or unsubstituted C₁-C₆ amino, or —CₓF₂ₓ₊₁, and x is an integer of 1 to 3; and p is an integer of 1 to 3

In general formula (I), when m is equal to or greater than 2, two or more R³'s may be connected to each other to form a C₃-C₈ aromatic ring or a nitrogen-containing heteroaromatic ring. Similarly, when 1 is equal to or greater than 2, two or more R²'s may be connected to each other to form a C₃-C₈ aromatic ring or a nitrogen-containing heteroaromatic ring. When n is equal to or greater than 2, two or more R⁴'s may be connected to each other to form a C₃-C₈ aromatic ring or a nitrogen-containing heteroaromatic ring. When p is equal to or greater than 2, two or more R⁶'s may be connected to each other to form a C₃-C₈ aromatic ring or a nitrogen-containing heteroaromatic ring.

The aromatic ring or nitrogen-containing heteroaromatic ring may include aromatic hydrocarbon or aromatic heterocycle. Specific examples of the aromatic ring or nitrogen-containing heteroaromatic ring include benzene, pyridine, pyrazine, pyrimidine, pyridazine, triazine, pyrrole, furan, thiophene, selenophene, tellurophene, imidazole, thiazole, selenazole, tellurazole, thiadiazole, oxadiazole, and pyrazole.

In an embodiment, when b is 0, the two tridentate chelates of the iridium complex both have a complete conjugated structure.

In an embodiment, when b is 1, the right tridentate chelate of the iridium complex has an extended conjugation, and the left tridentate chelate has a partially interrupted conjugation.

In an embodiment, the iridium complex has a structure represented by general formula (IA):

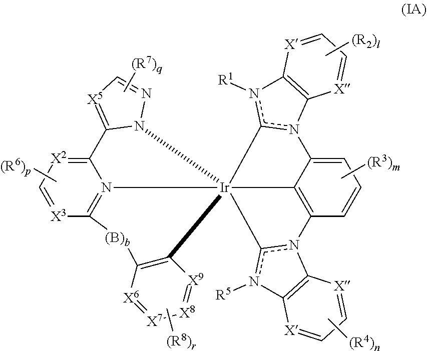

wherein
   X⁵, X⁶, X⁷, X⁸, and X⁹ are each independently carbon or nitrogen; R⁷ and R⁸ are each independently hydrogen, substituted or unsubstituted C₁-C₁₂ alkyl, substituted or unsubstituted C₁-C₆ alkoxy, substituted or unsubstituted C₆-C₁₂ aryl, or —CₓF₂ₓ₊₁, and x is an integer of 1 to 3; q is an integer of 1 to 2; r is an integer of 1 to 4; when q is equal to or greater than 2, two or more R⁶'s may be connected to each other to form a C₃-C₈ aromatic ring or a nitrogen-containing heteroaromatic ring; and when r is equal to or greater than 2, two or more R⁷'s may be connected to each other to form a C₃-C₈ aromatic ring or a nitrogen-containing heteroaromatic ring.

In an embodiment, the iridium complex has a structure represented by one of formula (IA-1) to formula (IA-144):

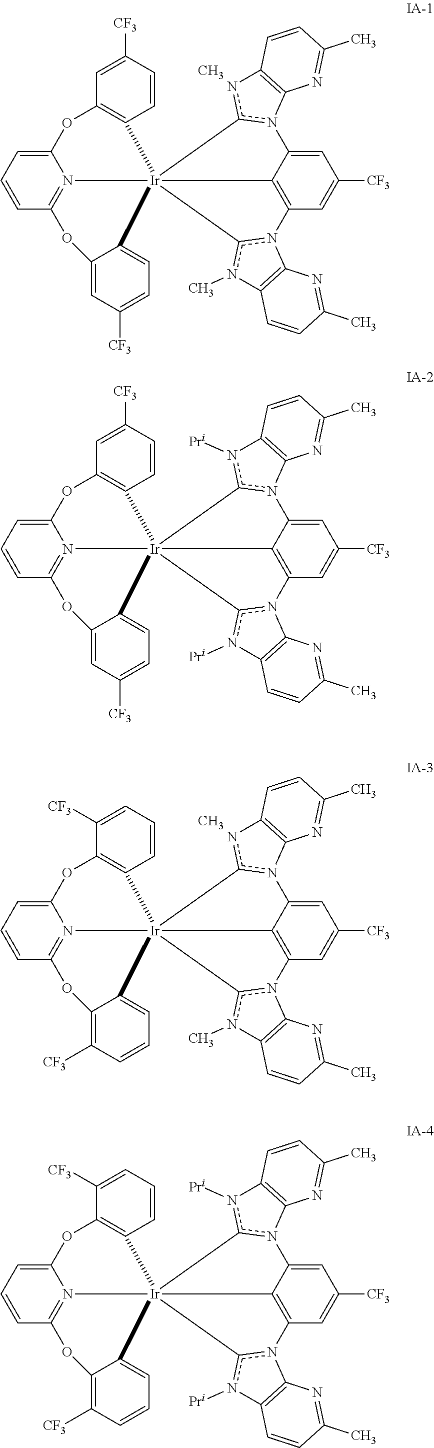

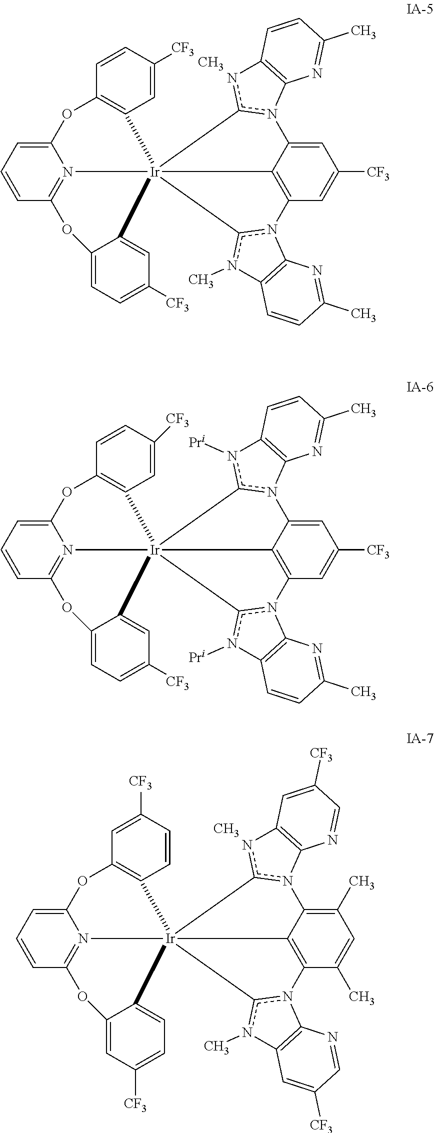

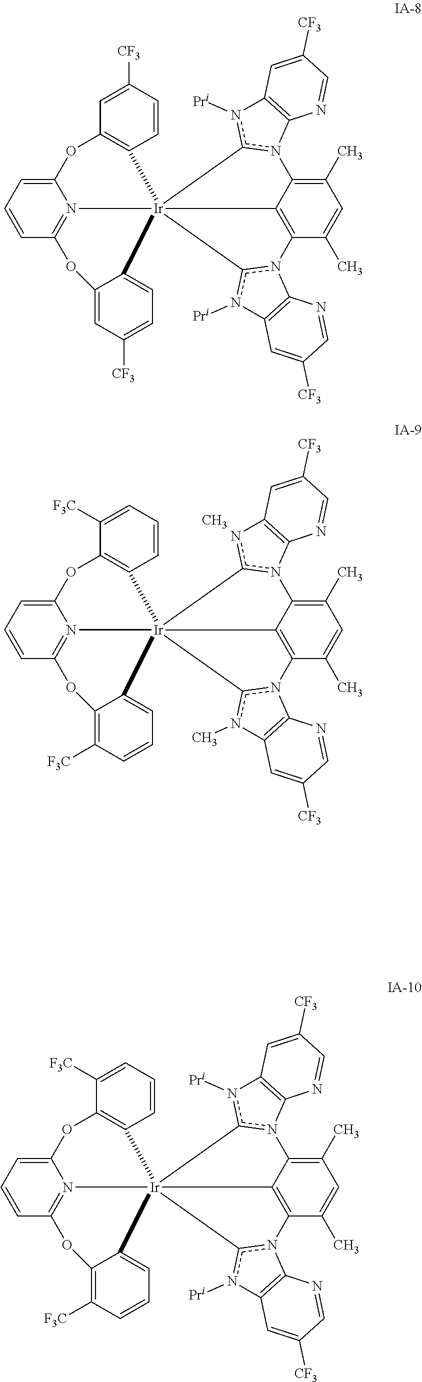

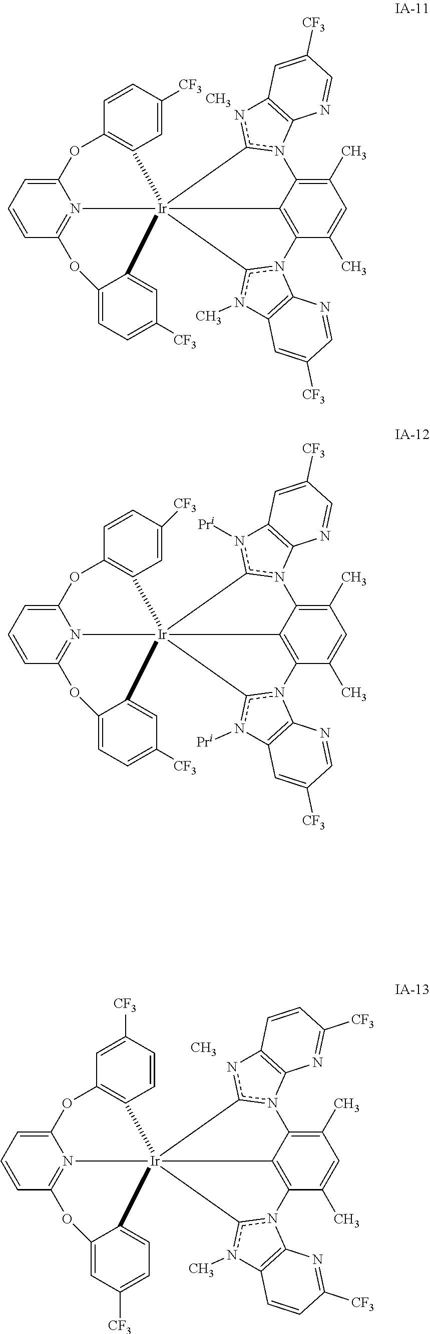

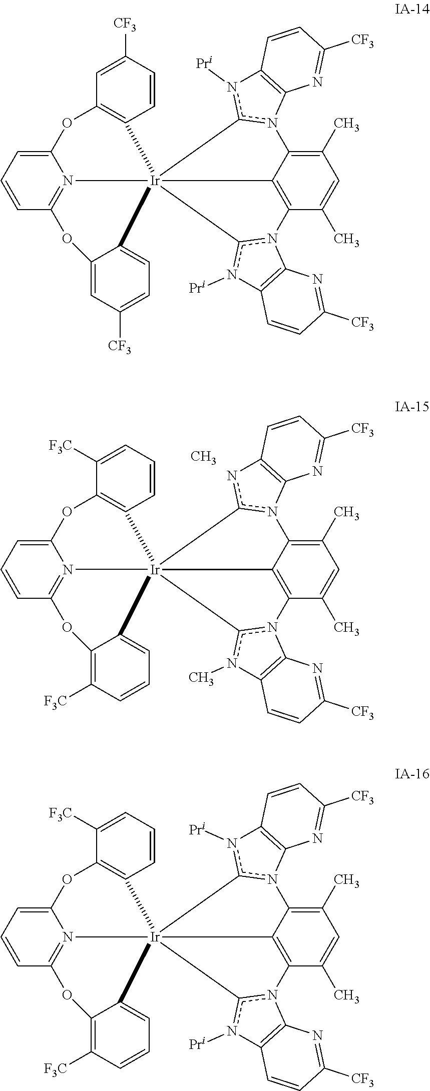

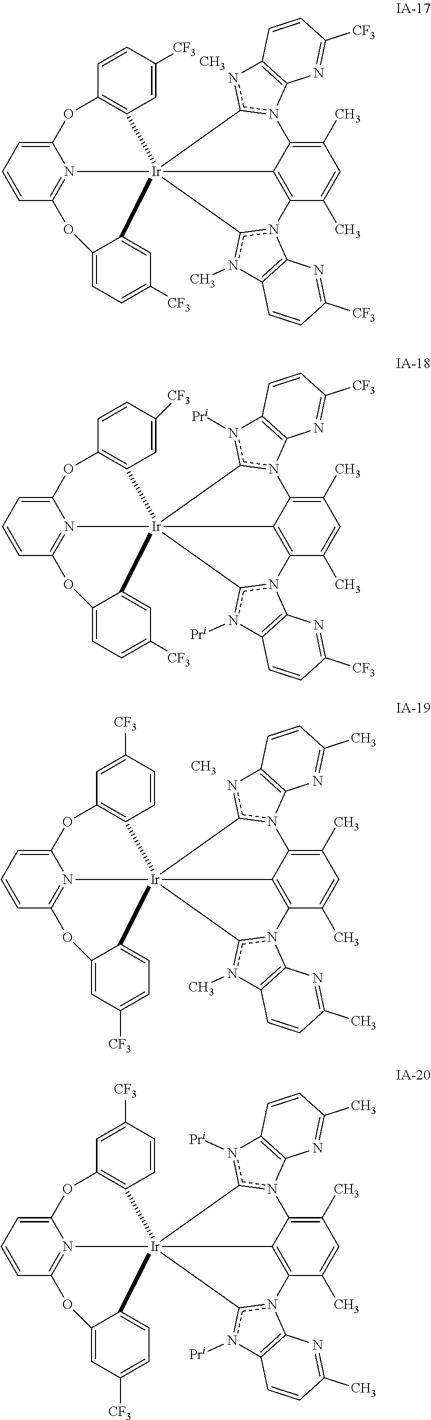

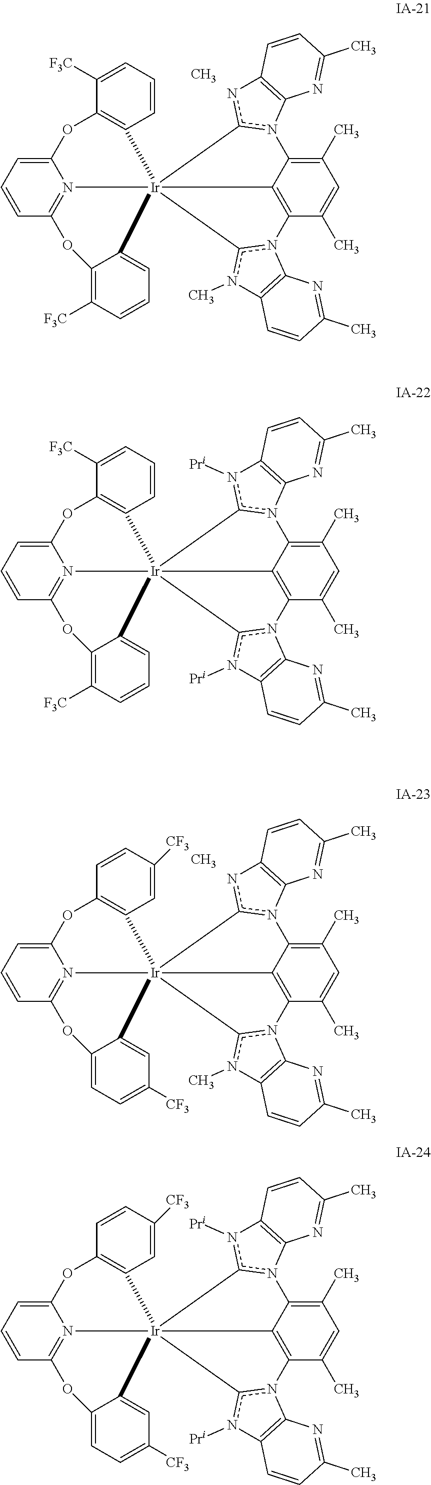

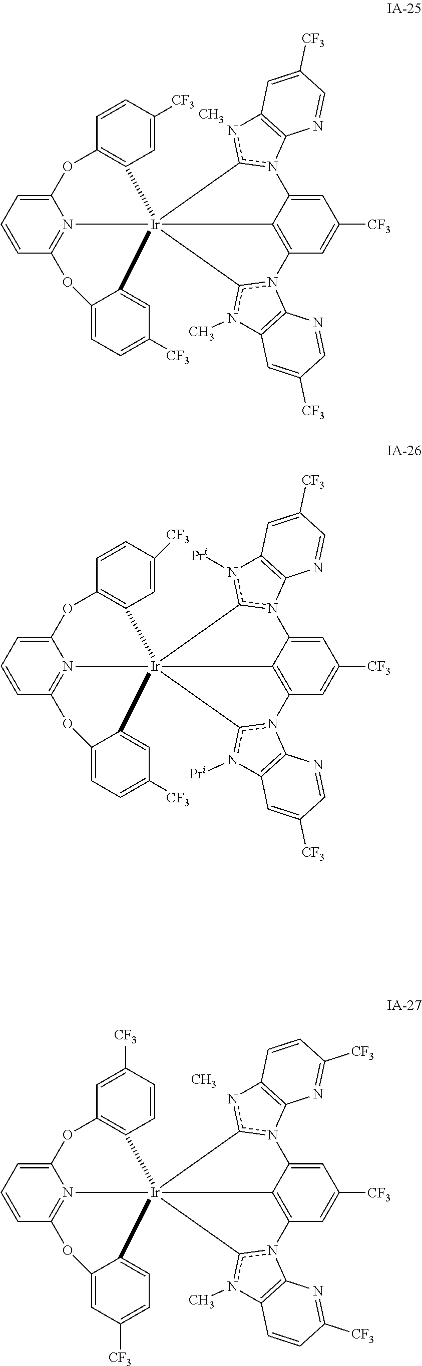

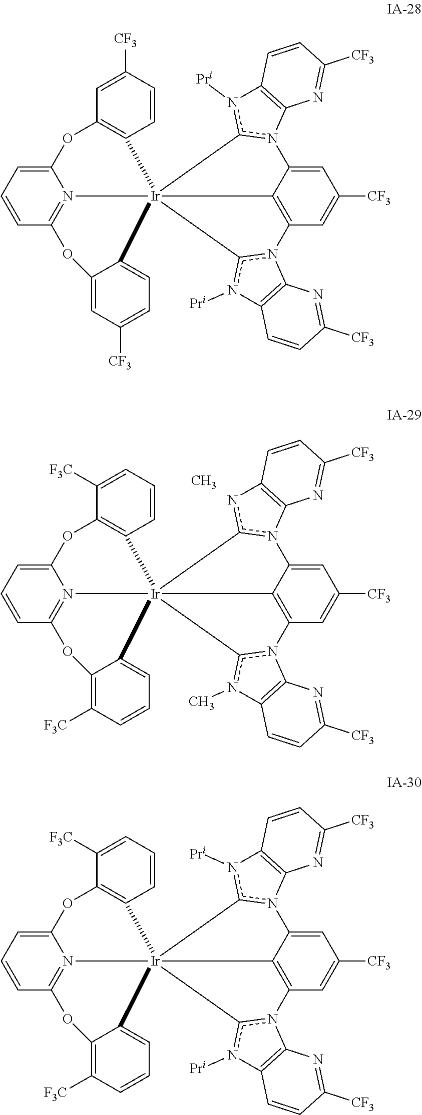

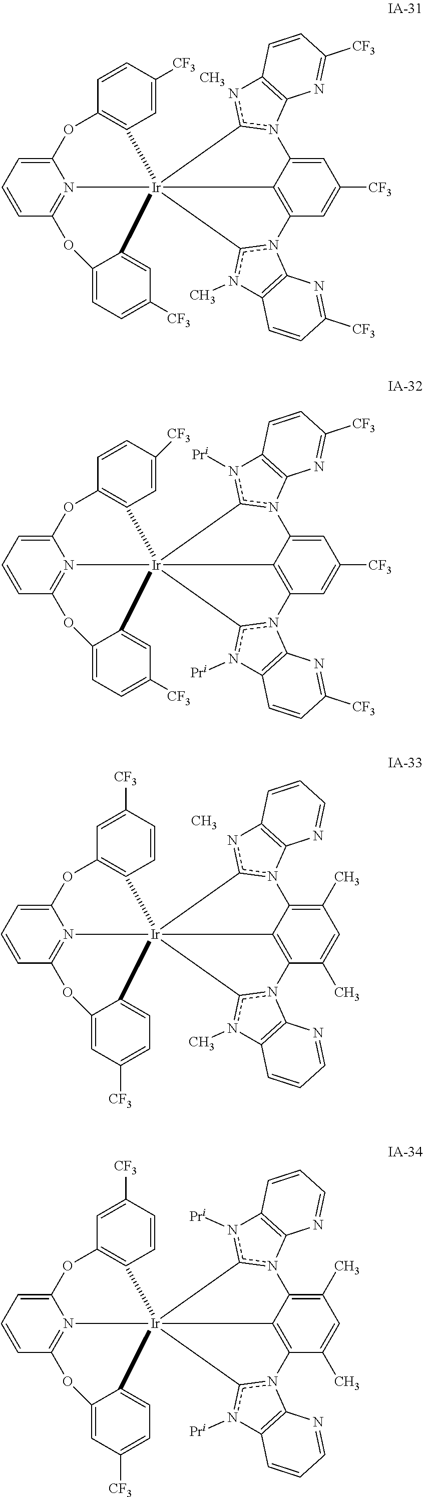

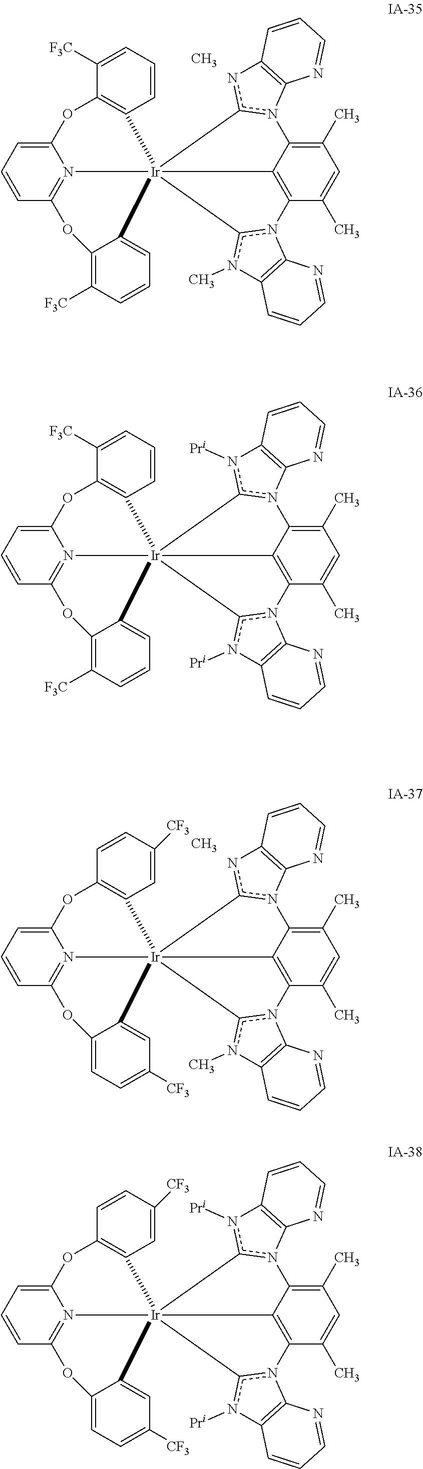

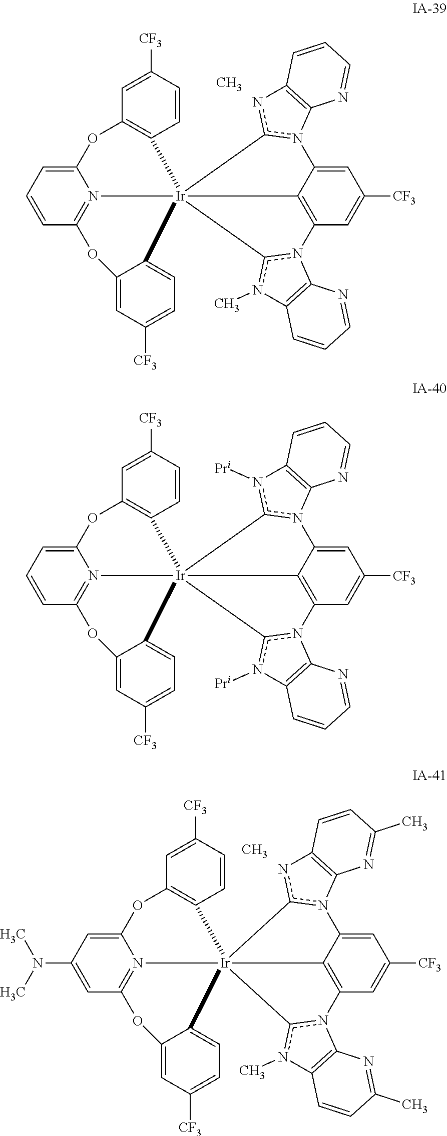

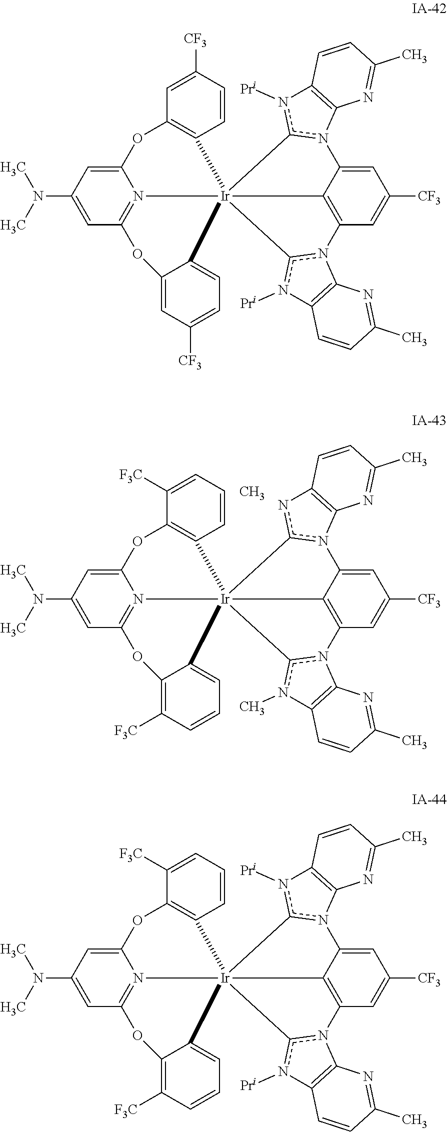

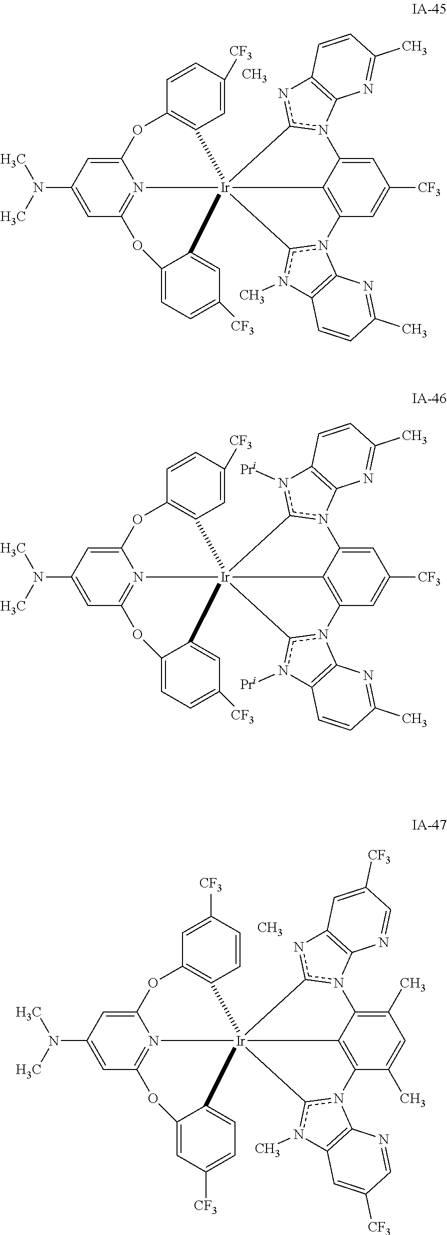

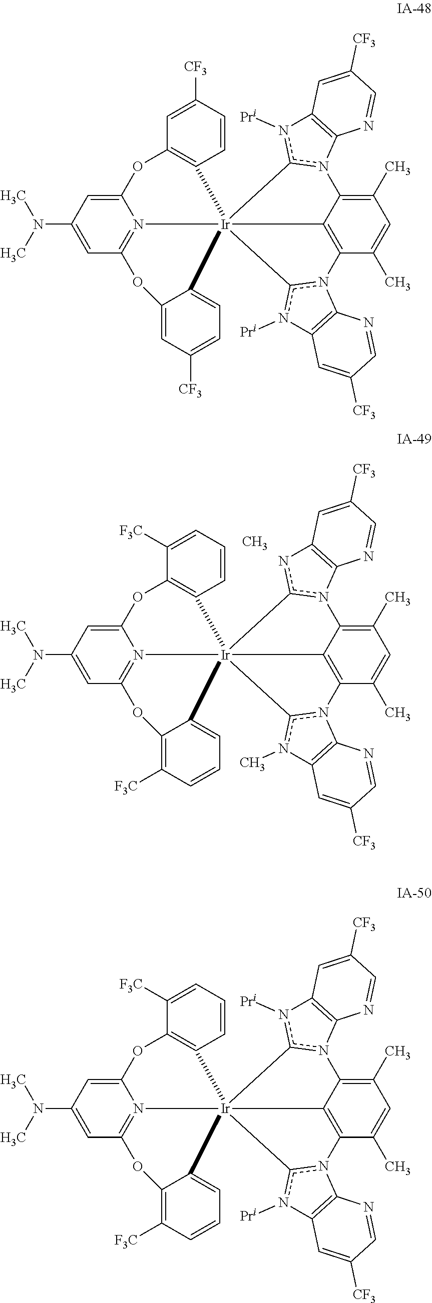

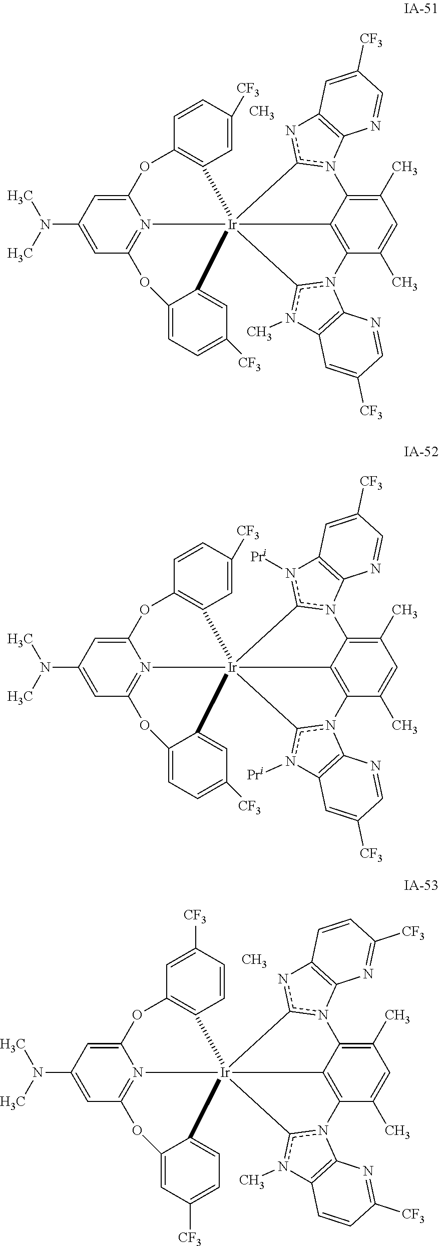

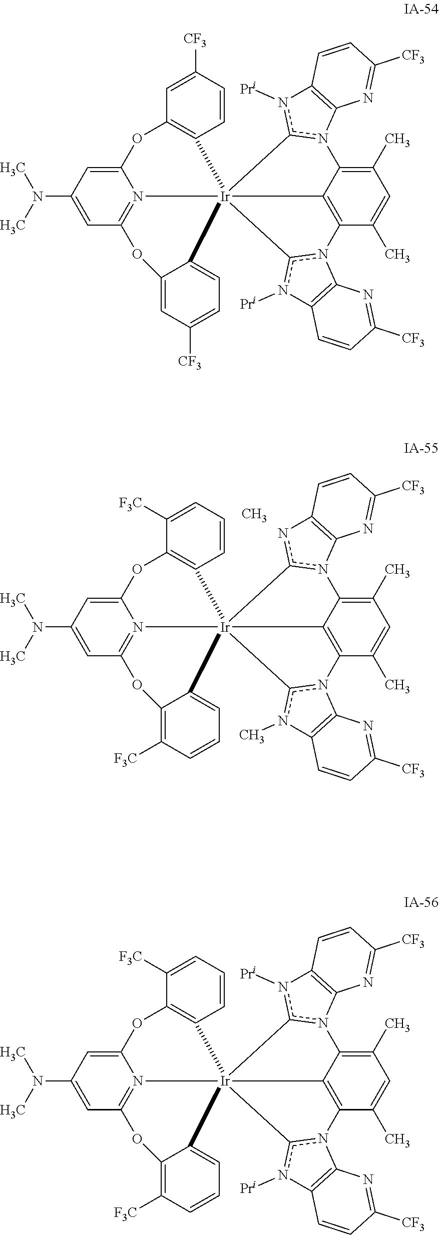

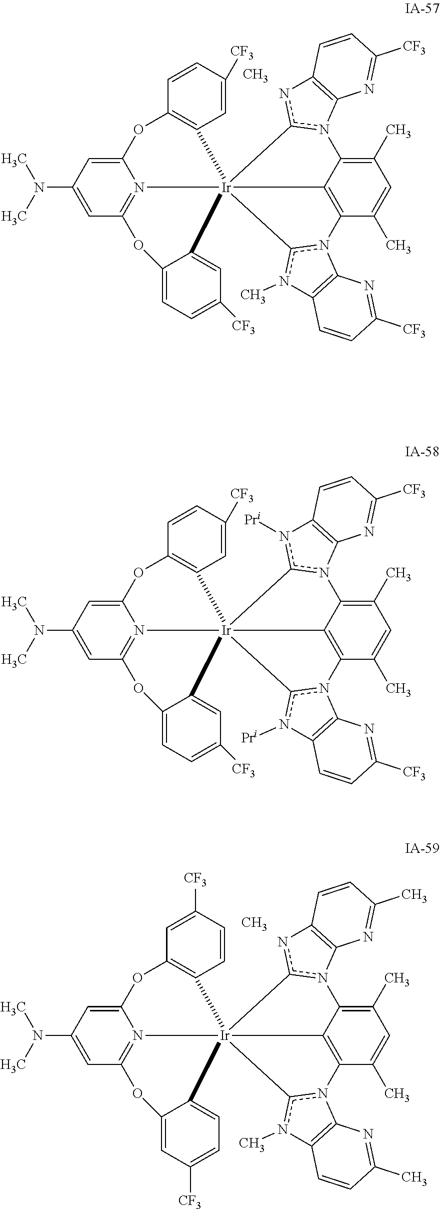

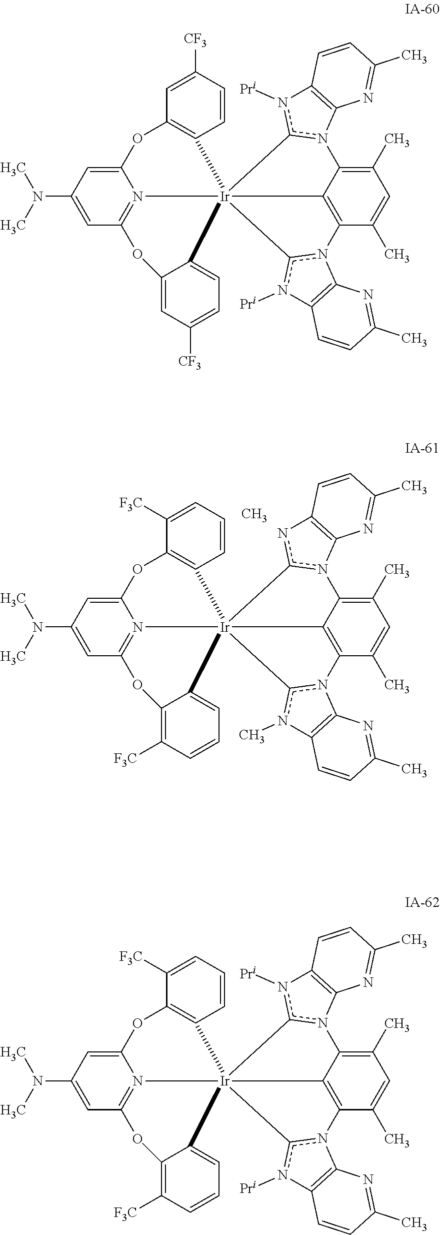

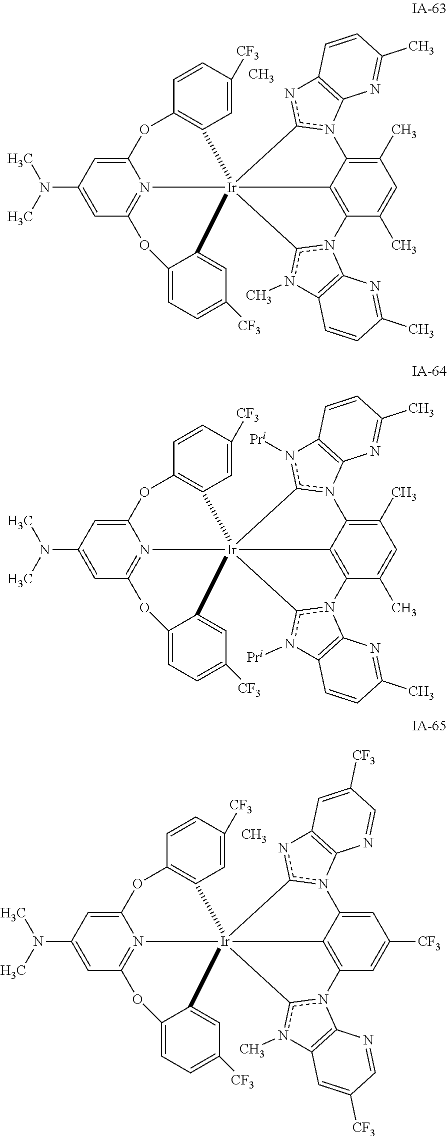

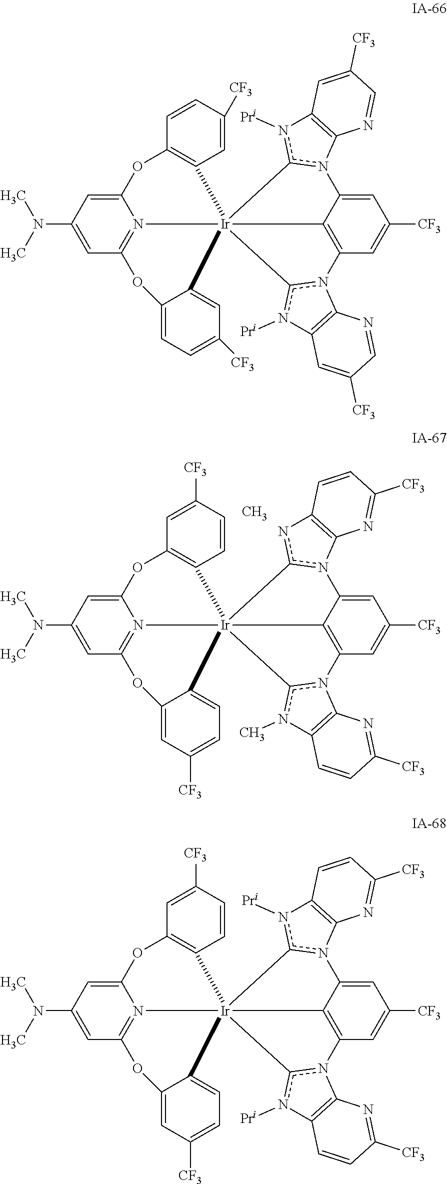

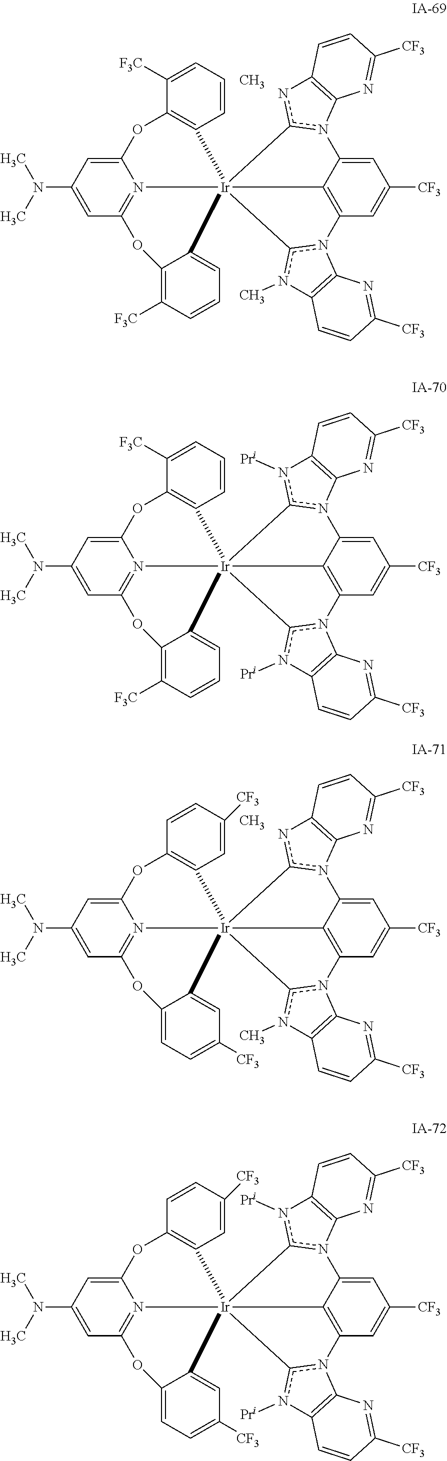

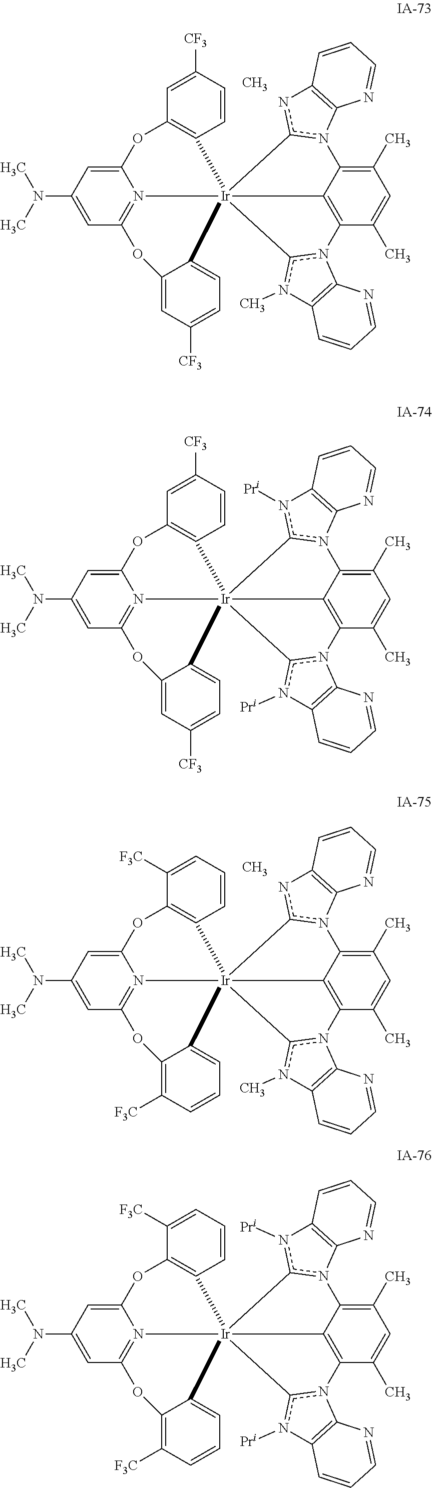

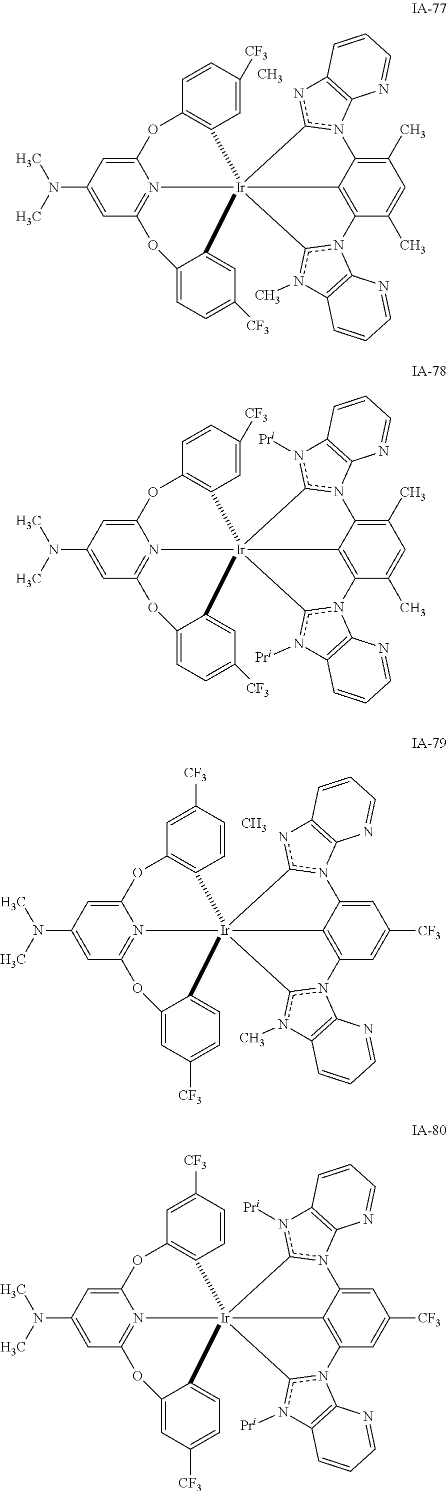

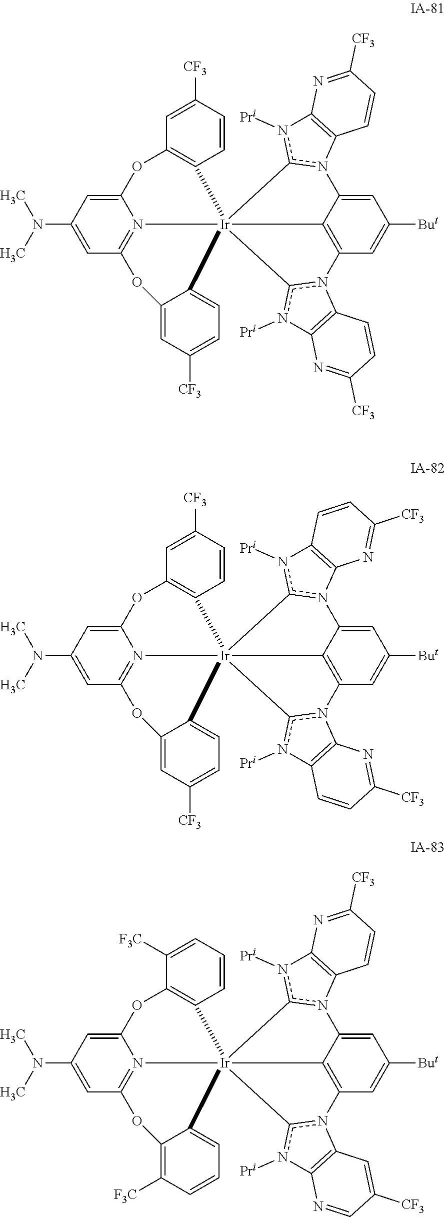

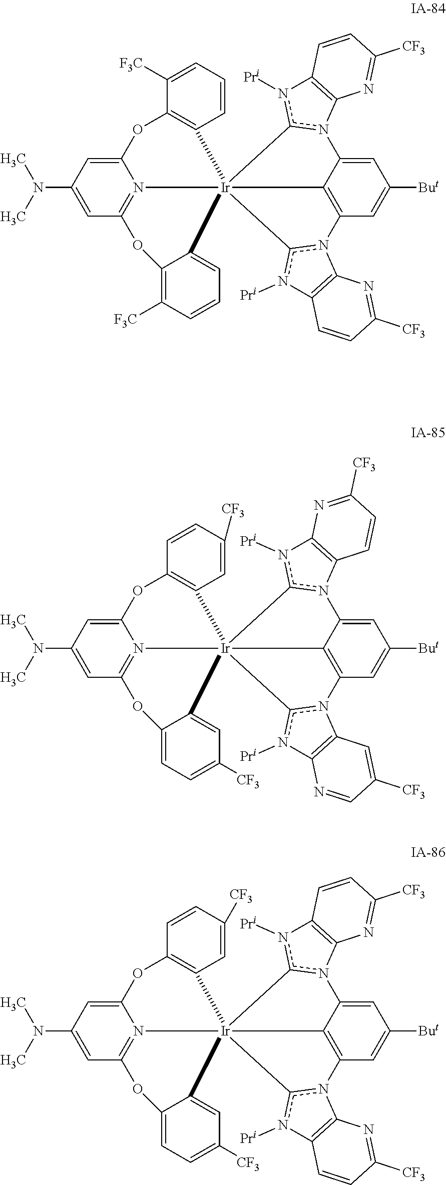

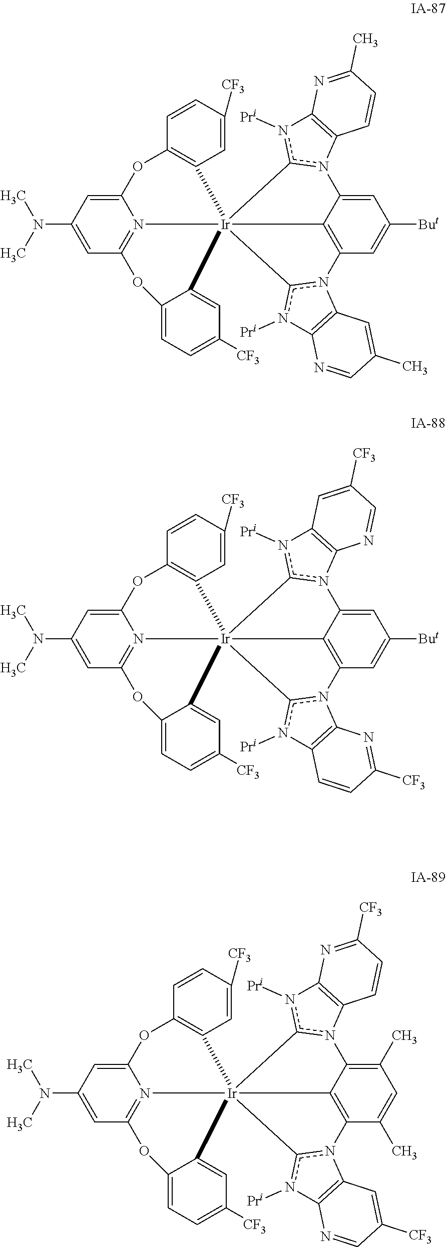

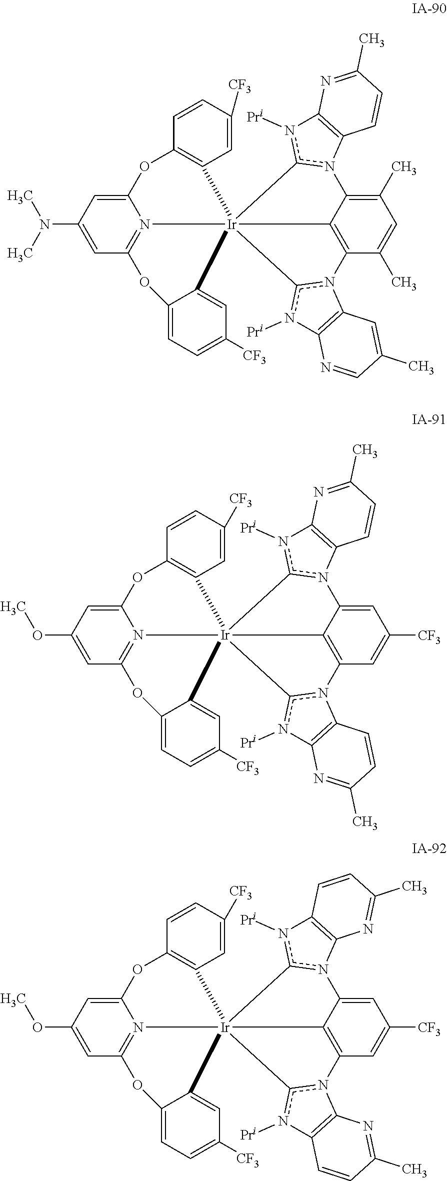

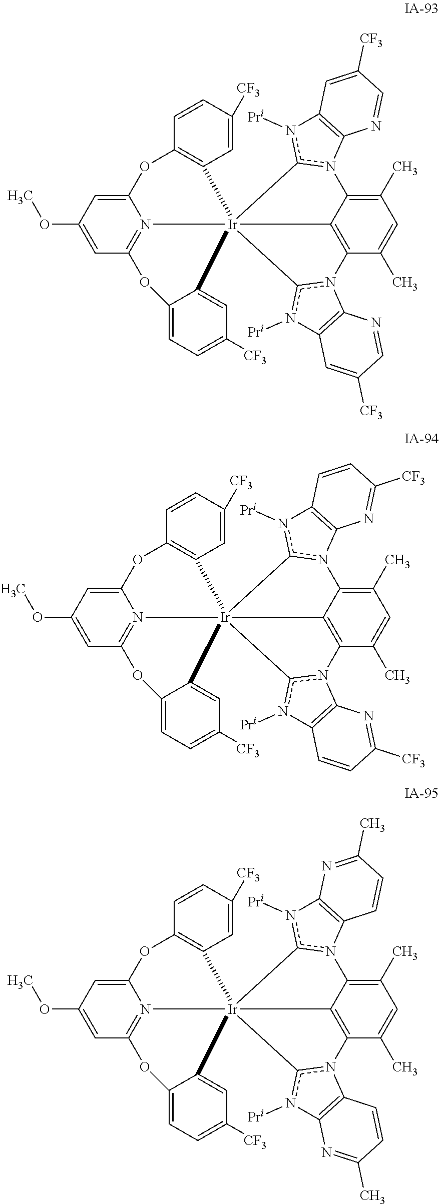

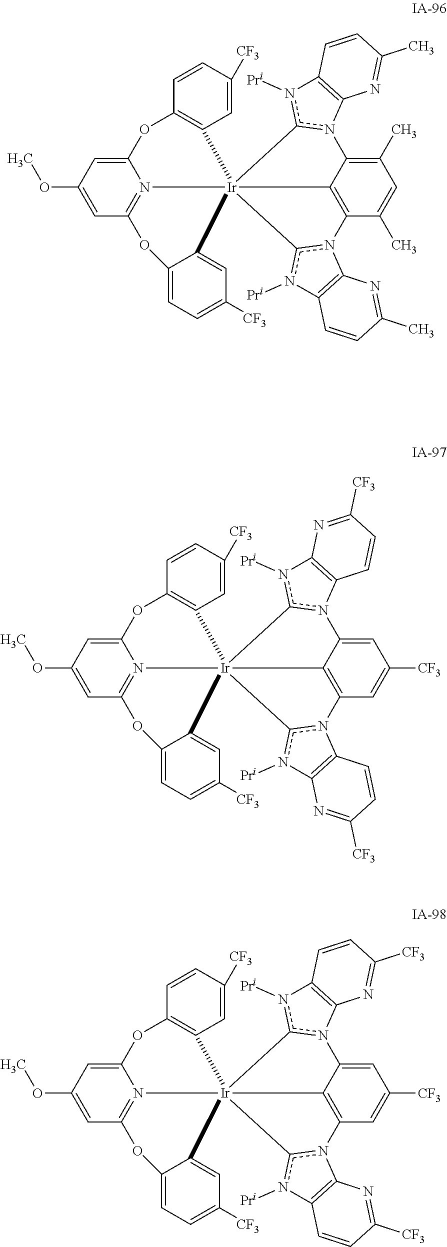

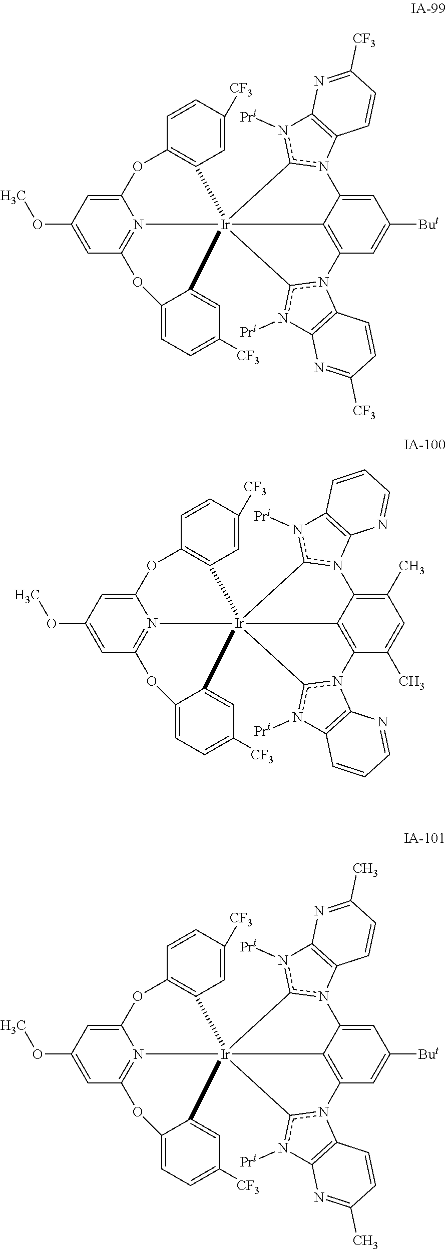

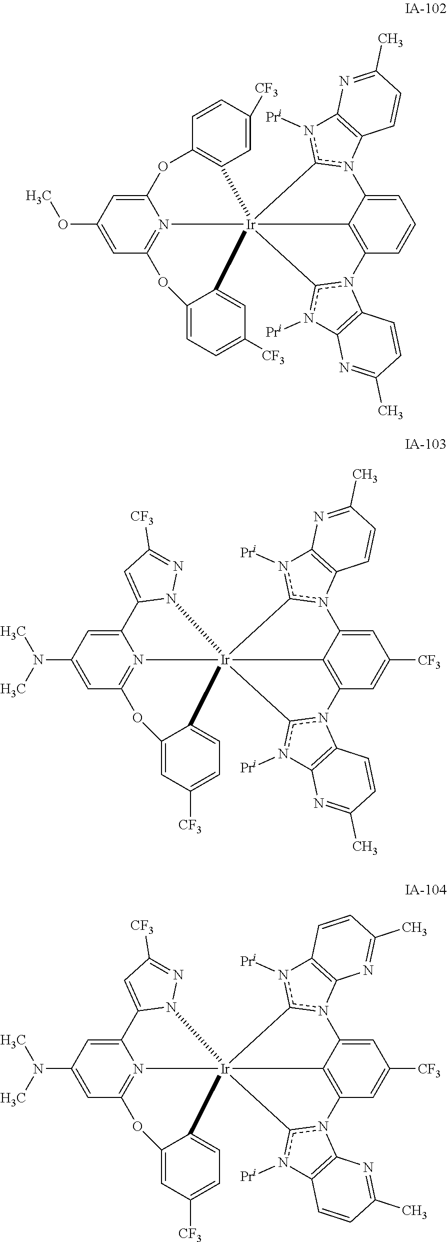

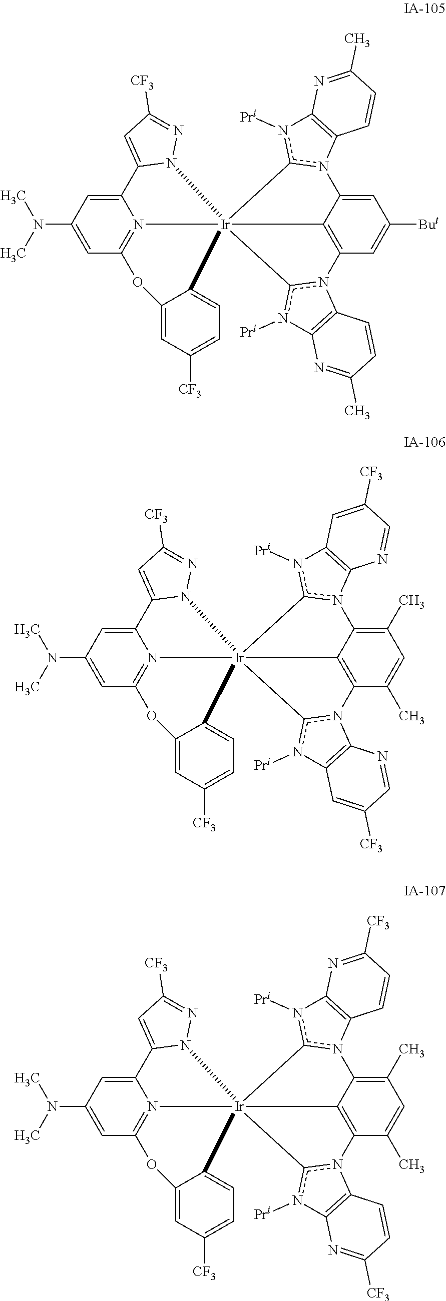

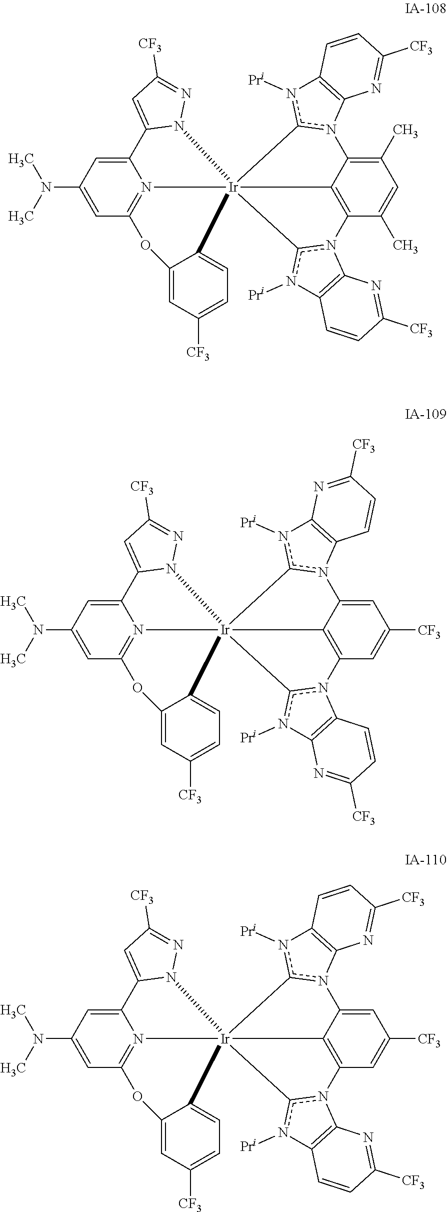

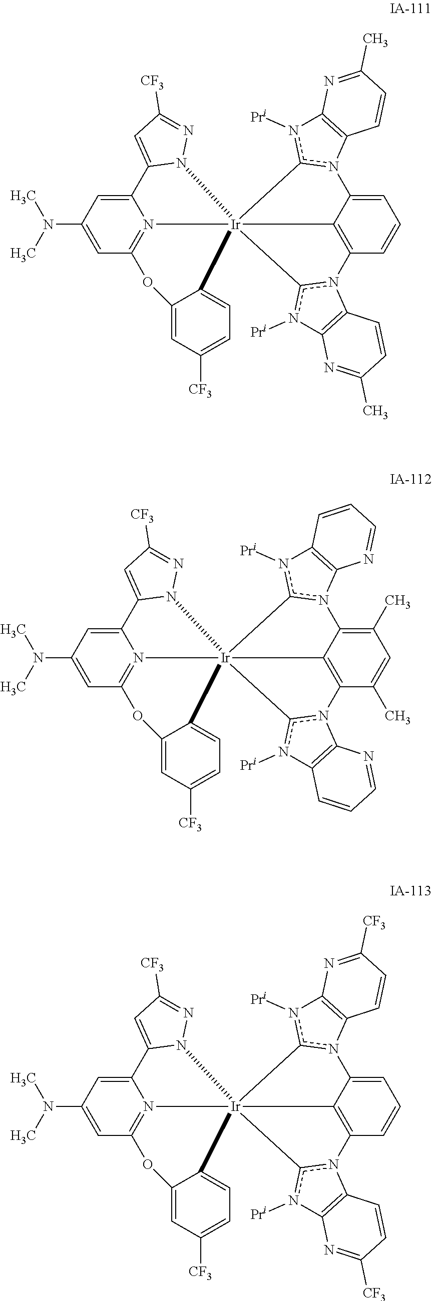

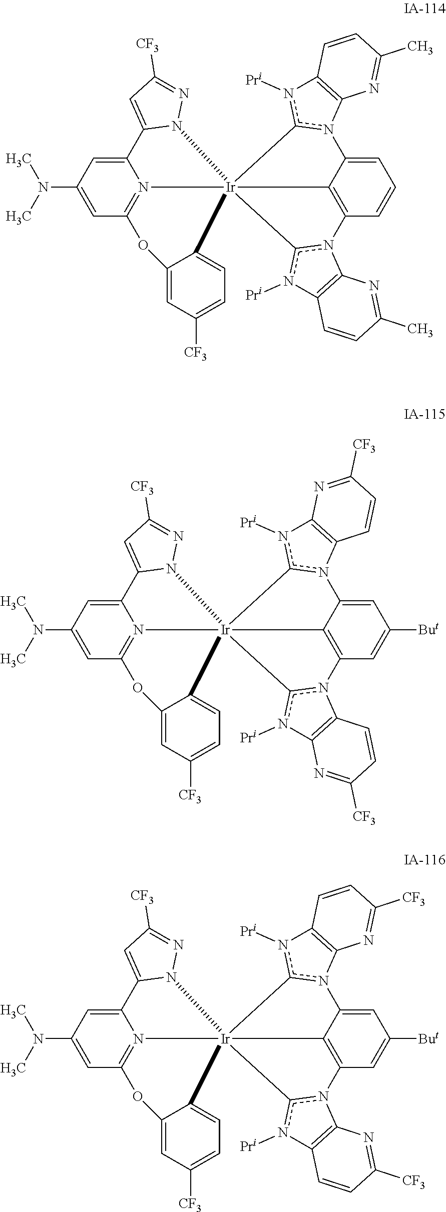

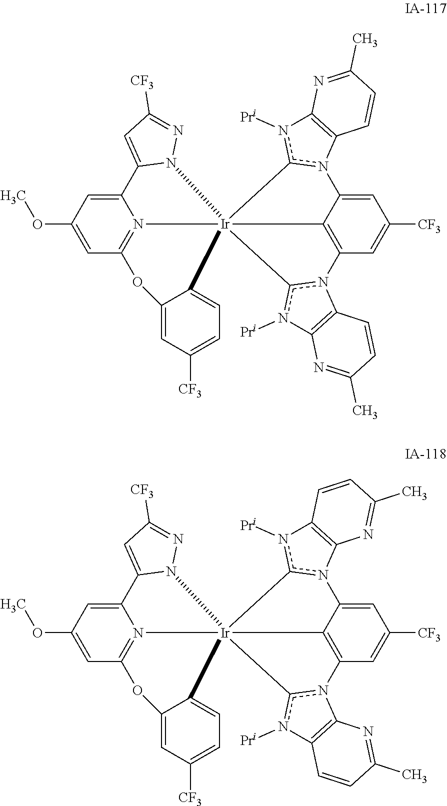

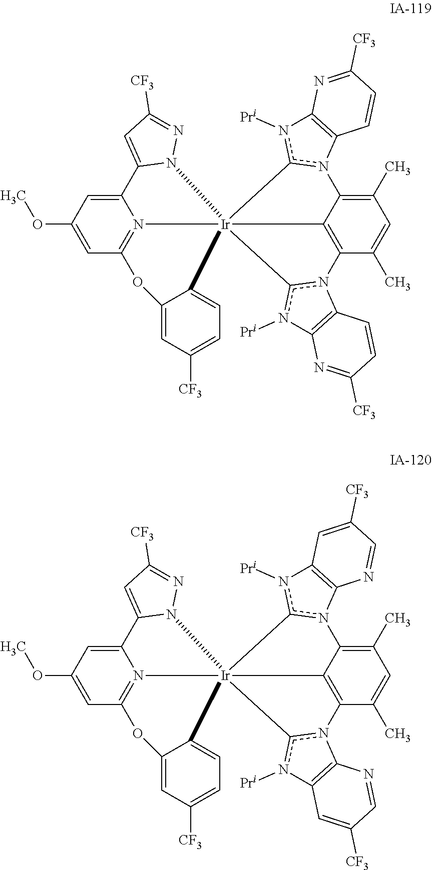

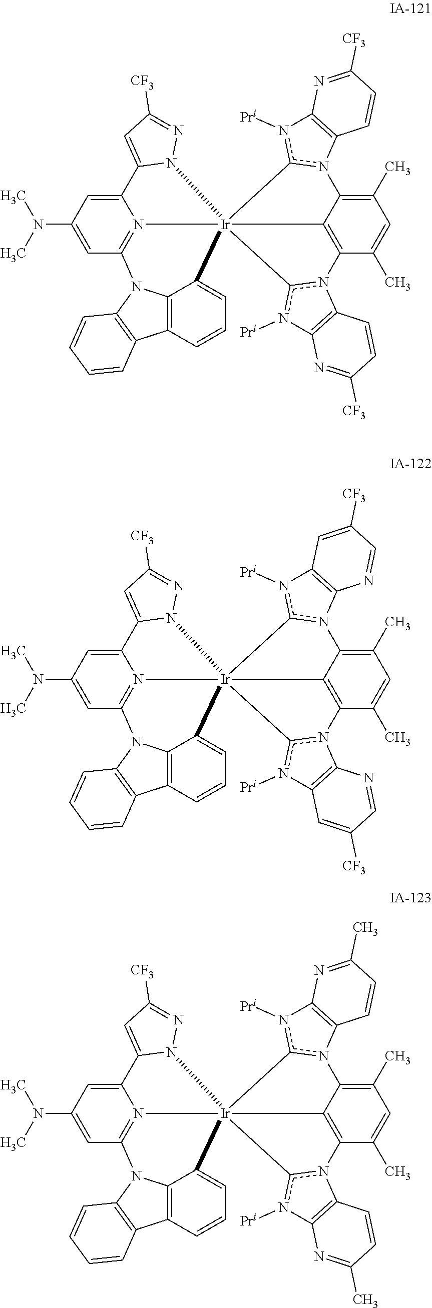

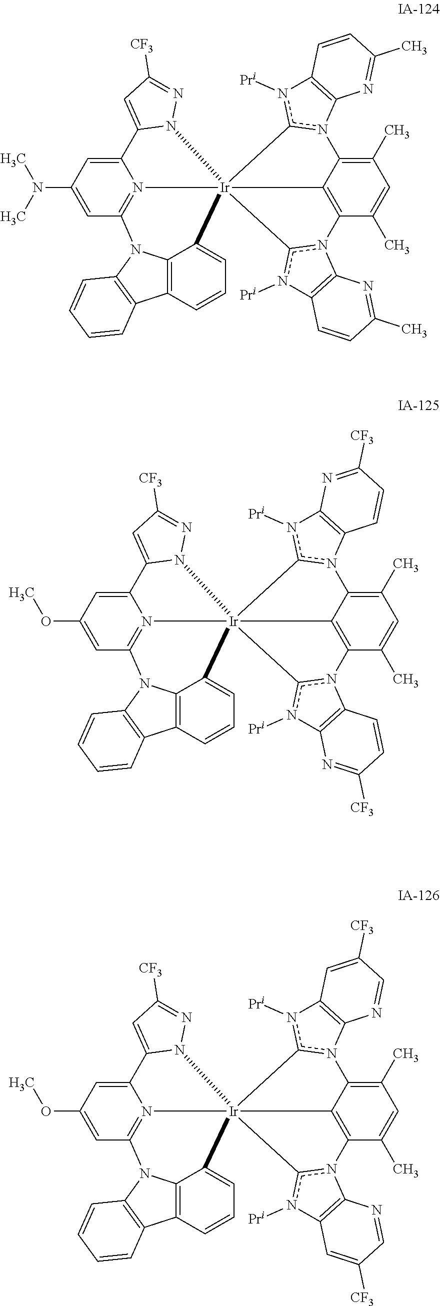

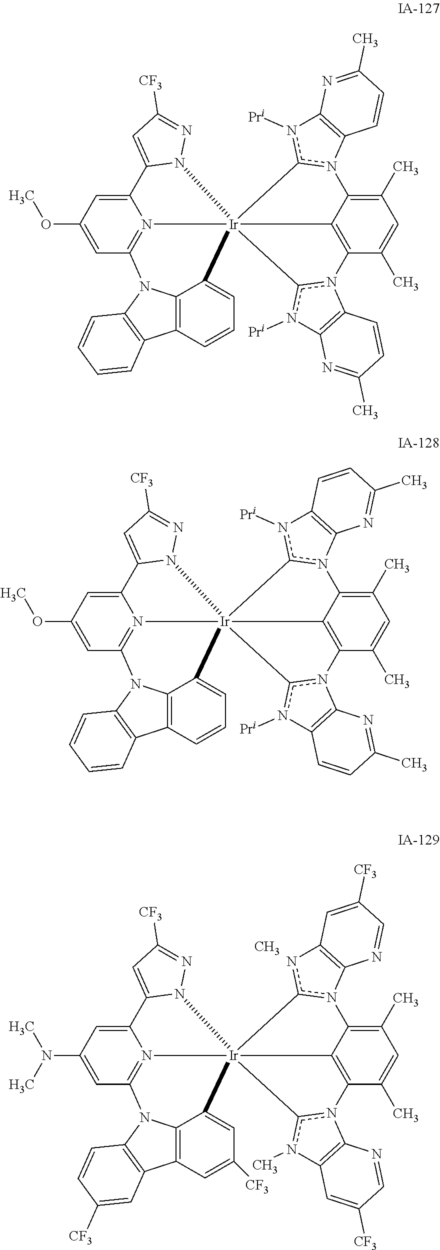

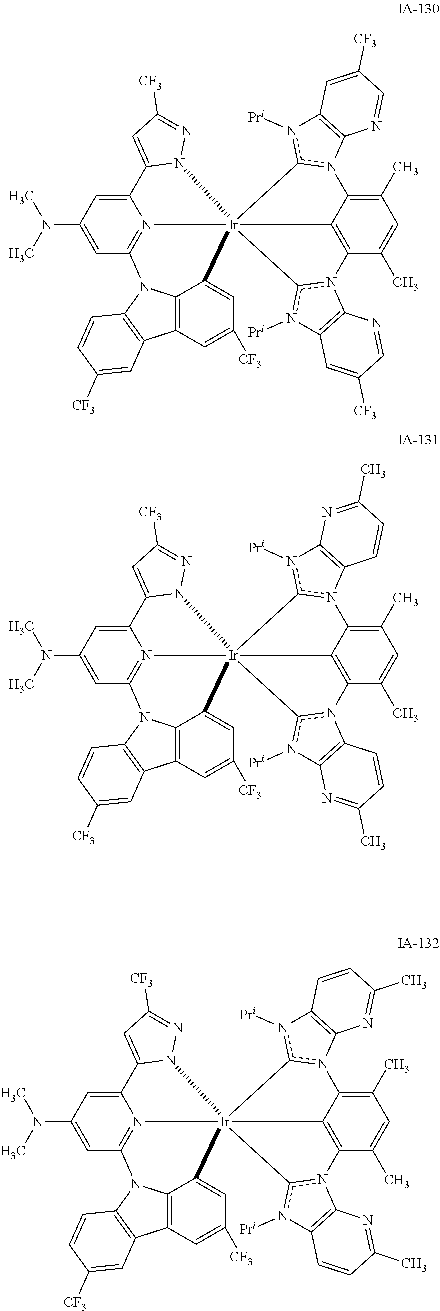

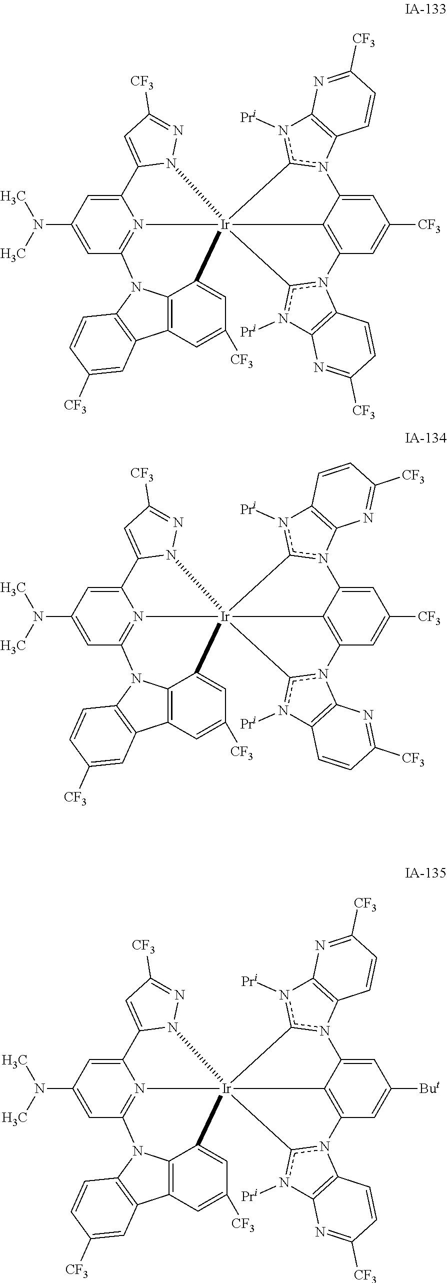

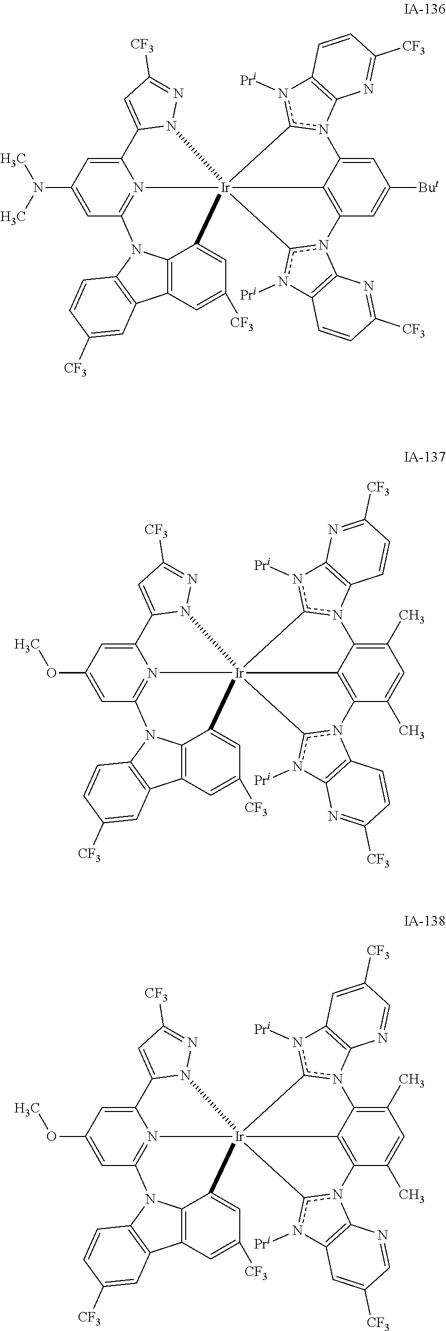

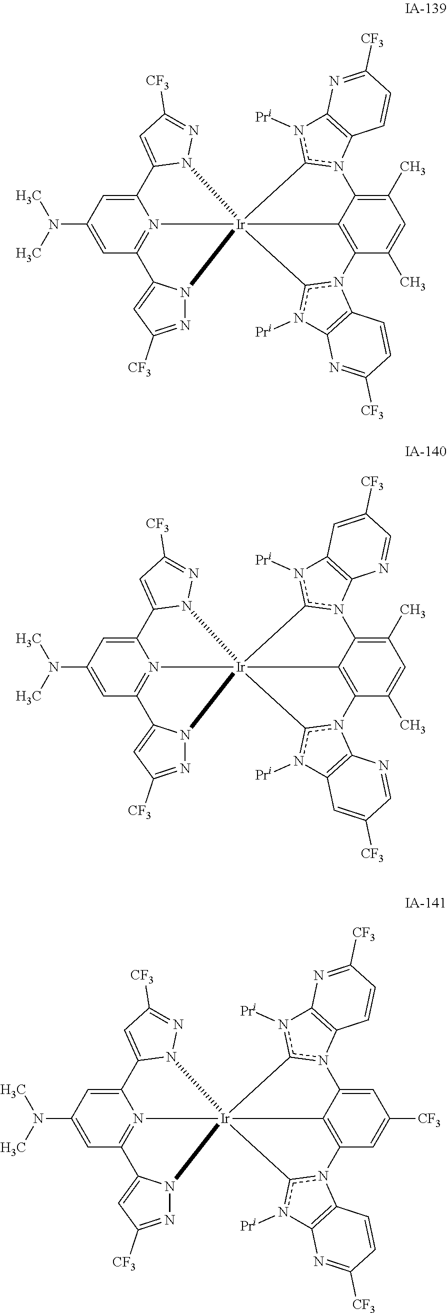

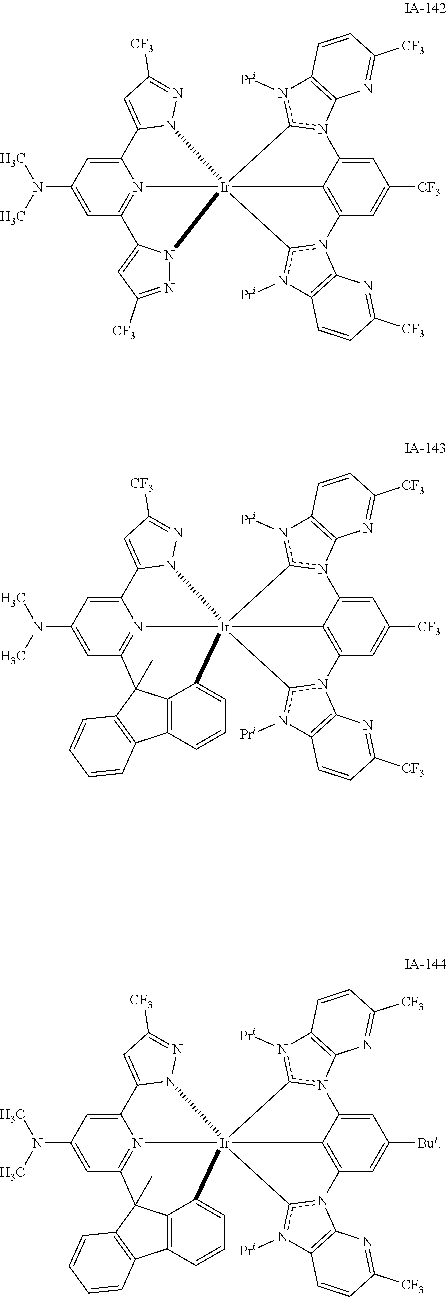

In an embodiment, the emission wavelength of the iridium complex occurs in the region between 430 nm and 550 nm, such as between 390 nm and 480 nm.

The invention provides an organic light-emitting diode including two electrodes and a light-emitting layer disposed between the two electrodes, wherein the light-emitting layer includes the iridium complex.

The invention provides an iridium complex having a structure represented by general formula (I-1) or (1-2):

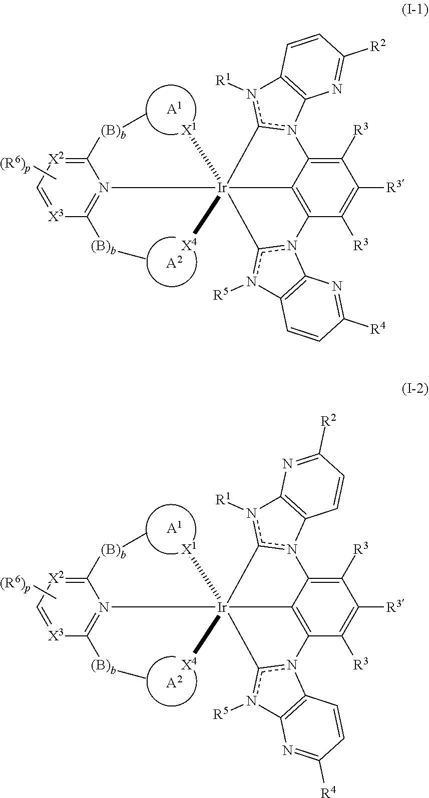

wherein X¹, X², X³, and X⁴ are each independently carbon or nitrogen; R¹ and R⁵ are each independently substituted or unsubstituted C₁-C₁₂ alkyl; R², R³, and R⁴ are each independently hydrogen, substituted or unsubstituted C₁-C₁₂ alkyl, or substituted or unsubstituted C₆-C₁₂ aryl; R^{3′} is an electron withdrawing group or an electron donating group, wherein the electron withdrawing group includes —CₘF₂ₘ₊₁, m is an integer of 0 to 3, and the electron donating group includes methyl, tert-butyl, or C₁-C₆ alkoxy; A¹ and A² are each independently an unsaturated 5-membered ring or an unsaturated 6-membered ring; B is —O—, —NR—, or —CR₂—, R is hydrogen or substituted or unsubstituted C₁-C₁₂ alkyl, and —NR— or —CR₂— may optionally be connected to A² to form a substituted or unsubstituted C₉-C₁₄ aromatic ring or a nitrogen-containing heteroaromatic ring; b is 0 or 1; R⁶ is hydrogen, fluorine, substituted or unsubstituted C₁-C₁₂ alkyl, substituted or unsubstituted C₁-C₆ alkoxy, substituted or unsubstituted C₆-C₁₂ aryl, substituted or unsubstituted C₁-C₆ amino, or —CₓF₂ₓ₊₁, and x is an integer of 1 to 3; p is an integer of 1 to 3; and when p is equal to or greater than 2, two or more R⁶'s may be connected to each other to form a C₃-C₈ aromatic ring or a nitrogen-containing heteroaromatic ring.

In general formula (I-1), at least one of R² and R³ is not hydrogen and at least one of R³ and R⁴ is not hydrogen so as to create steric encumbrance to protect the N atoms between R² and R³ or between R³ and R⁴, thereby stabilizing the structure of the iridium complex and increasing the thermal decomposition temperature.

In general formula (I-2), both R² and R⁴ are not hydrogen, and adjacent N atoms are protected by steric encumbrance, thereby stabilizing the structure of the iridium complex and increasing the thermal decomposition temperature.

In general formula (I-1) or (1-2), R^{3′} is an electron withdrawing group or an electron donating group, which may change the light-emitting wavelength of the iridium complex.

In an embodiment, the iridium complex has a structure represented by general formula (I-1A) or (I-2A):

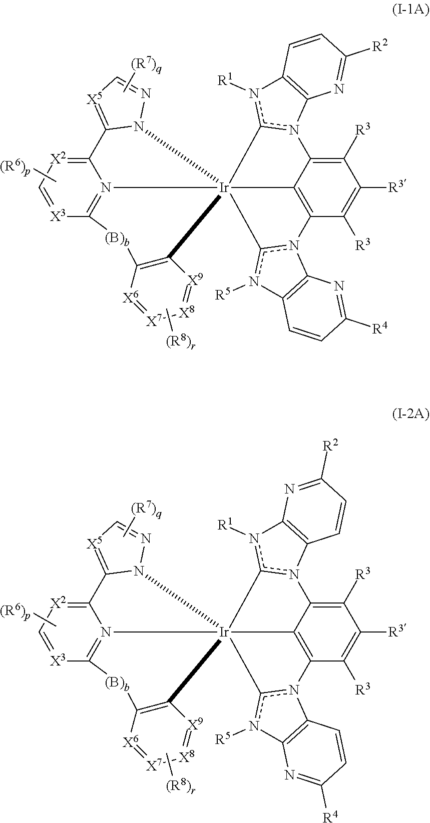

wherein X⁵, X⁶, X⁷, X⁸, and X⁹ are each independently carbon or nitrogen; R⁷ and R⁸ are each independently hydrogen, substituted or unsubstituted C₁-C₁₂ alkyl, substituted or unsubstituted C₁-C₆ alkoxy, substituted or unsubstituted C₆-C₁₂ aryl, or —CₓF₂ₓ₊₁, and x is an integer of 1 to 3; q is an integer of 1 to 2; r is an integer of 1 to 4; when q is equal to or greater than 2, two or more R⁶'s may be connected to each other to form a C₃-C₈ aromatic ring or a nitrogen-containing heteroaromatic ring; and when r is equal to or greater than 2, two or more R⁷'s may be connected to each other to form a C₃-C₈ aromatic ring or a nitrogen-containing heteroaromatic ring.

### [Structure of Nitrogen-Containing Tridentate Carbene Chelate]

The invention provides a nitrogen-containing tridentate carbene chelate having a structure represented by general formula (II):

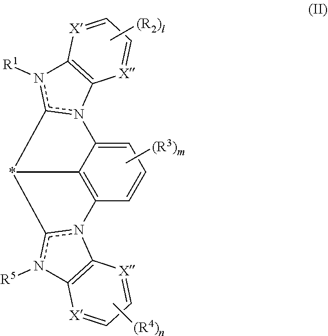

wherein
* represents a bonding site;
one of X′ and X″ is nitrogen, and the other is carbon; R¹ and R⁵ are each independently substituted or unsubstituted C₁-C₁₂ alkyl; R², R³, and R⁴ are each interpedently hydrogen, substituted or unsubstituted C₁-C₁₂ alkyl, substituted or unsubstituted C₆-C₁₂ aryl, or —CₘF₂ₘ₊₁, and m is an integer of 0 to 3; 1, m, and n are each independently an integer of 1 to 3; when 1 is equal to or greater than 2, two or more R²'s may be connected to each other to form a C₃-C₈ aromatic ring or a nitrogen-containing heteroaromatic ring; when m is equal to or greater than 2, two or more R³'s may be connected to each other to form a C₃-C₈ aromatic ring or a nitrogen-containing heteroaromatic ring; and when n is equal to or greater than 2, two or more R⁴'s may be connected to each other to form a C₃-C₈ aromatic ring or a nitrogen-containing heteroaromatic ring.

In an embodiment, the nitrogen-containing tridentate carbene chelate has a structure represented by general formula (II-1) or (II-2):

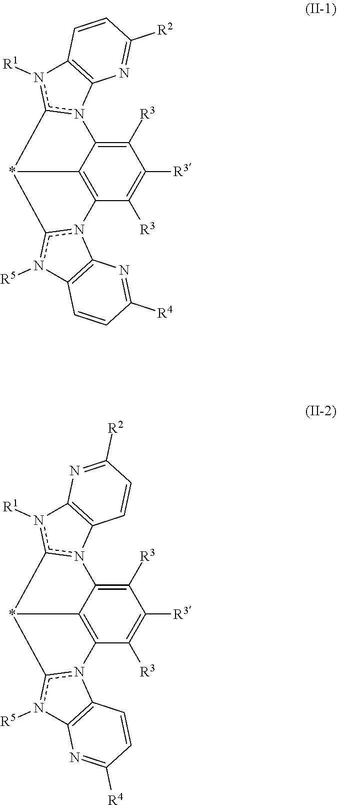

wherein R², R³, and R⁴ are each independently hydrogen, substituted or unsubstituted C₁-C₁₂ alkyl, or substituted or unsubstituted C₆-C₁₂ aryl; R^{3′} is an electron withdrawing group or an electron donating group, the electron withdrawing group includes —CₘF₂ₘ₊₁, m is an integer of 0 to 3, and the electron donating group includes methyl, tert-butyl, or C₁-C₆ alkoxy.

In general formula (II-1), at least one of R² and R³ is not hydrogen and at least one of R³ and R⁴ is not hydrogen, and the N atoms between R² and R³ or between R³ and R⁴ are protected by steric encumbrance, thereby stabilizing the structure of the iridium complex and increasing the thermal decomposition temperature.

In general formula (II-2), both R² and R⁴ are not hydrogen, and adjacent N atoms are protected by steric encumbrance, thereby stabilizing the structure of the iridium complex and increasing the thermal decomposition temperature.

### [Preparation of Mono-Negative Charged Nitrogen-Containing Tridentate Carbene Chelate]

### Example 1

In an embodiment, taking complex (IA-40) of the invention as an example, the right-side carbene chelate of the complex of the invention may be prepared by the following reaction protocols.

The adopted synthetic procedures for (D1) were depicted in Scheme 1.

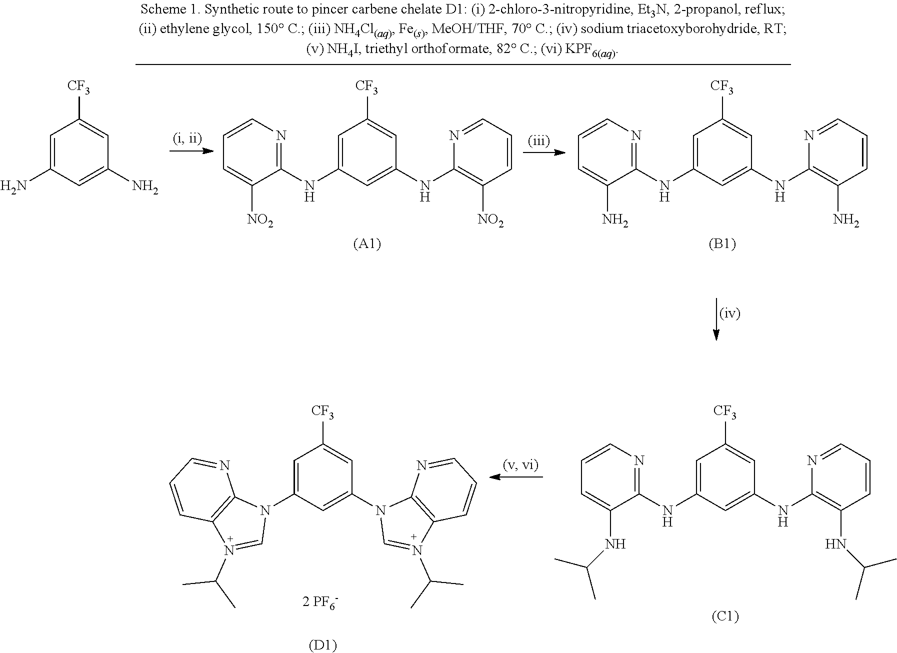

### (i, ii) Synthesis of N¹,N³-bis(3-nitropyridin-2-yl)-5-(trifluoromethyl)benzene-1,3-diamine (A1)

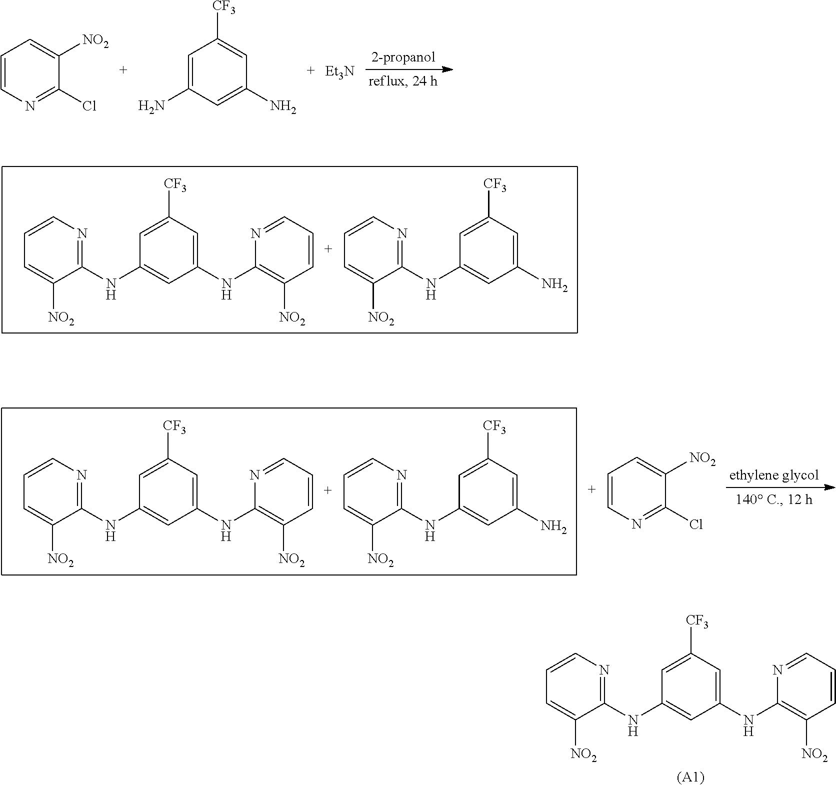

2-Chloro-3-nitropyridine (3.25 g, 20.5 mmol), 5-(trifluoromethyl)benzene-1,3-diamine (1.44 g, 8.20 mmol), and Et₃N (3.43 mL g, 24.6 mmol) in 45 mL of 2-propanol were refluxed for 24 h. Afterward, the reaction mixture was concentrated to dryness. To this mixture was added more 2-chloro-3-nitropyridine (1.95 g, 12.3 mmol) and ethylene glycol (45 mL). The mixture was heated at 150° C. for 12 h. After cooling down the mixture, the brown precipitate was collected and washed with deionized water to afford brown powder (A1); yield: 3.39 g, 98%.

Spectral data of A1: ¹H NMR (400 MHz, CDCl₃): δ 10.27 (s, 2H), 8.59˜8.55 (m, 4H), 8.28 (s, 1H), 7.82 (s, 2H), 6.96˜6.93 (m, 2H). ¹⁹F NMR (376 MHz, CDCl₃): δ −62.76 (s, 3 F).

### (iii) Synthesis of N²,N²′-(5-(trifluoromethyl)-1,3-phenylene)bis(pyridine-2,3-diamine) (B1)

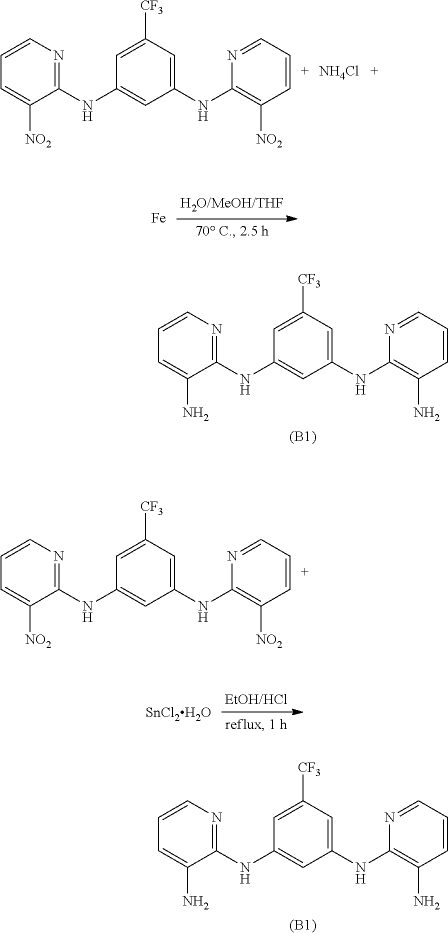

Iron fine powder (2.25 g, 40.3 mmol) was added to a cold (0° C.) solution of NH₄Cl (2.16 g, 40.3 mmol) in deionized water (60 mL). A solution of N¹,N³-bis(3-nitropyridin-2-yl)-5-(trifluoromethyl)benzene-1,3-diamine (A1) (3.39 g, 8.07 mmol) in a mixture of MeOH/THF (1:1, 60 mL) was added dropwise. The mixture was stirred at 70° C. for 2.5 h. After cooled to RT, it was filtered, washed and concentrated in vacuo. The aqueous phase obtained was extracted with ethyl acetate. Next, the organic solution was dried over anhydrous Na₂SO₄ and evaporated to dryness under vacuum to afford a brown powder (B1); yield: 2.86 g, 98%.

Spectral data of B1: ¹H NMR (400 MHz, CDCl₃): δ 7.82 (dd, J=4.9 Hz, 1.4 Hz, 2H), 7.46 (s, 1H), 7.06 (s, 2H), 7.01 (dd, J=7.6 Hz, 1.4 Hz, 2H), 6.79 (dd, J=7.6 Hz, 4.9 Hz, 2H), 6.43 (br, 2H), 3.47 (br, 4H). ¹⁹F NMR (376 MHz, CDCl₃): δ −62.76 (s, 3 F).

### (iv) Synthesis of N²,N²′-(5-(trifluoromethyl)-1,3-phenylene)bis(N³-isopropylpyridine-2,3-diamine) (C1)

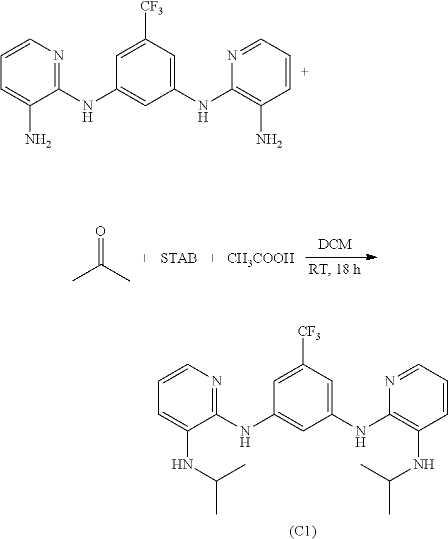

N²,N^{2′}-(5-(Trifluoromethyl)-1,3-phenylene)bis(pyridine-2,3-diamine) (B1) (1.75 g, 4.86 mmol) in acetone (1.08 mL, 14.6 mmol) was slowly added to a solution of acetic acid (1.11 mL, 19.4 mmol) and dichloromethane (25 mL). Sodium triacetoxyborohydride (3.09 g, 14.6 mmol) was added and stirred at RT for 18 h. After then, the mixture was quenched with 2N HCl_{(aq)} and, extracted with ethyl acetate. Ethyl acetate solution was washed with 1N NaOH_{(aq)} followed by brine, dried over anhydrous Na₂SO₄ and concentrated to dryness. The crude product was purified by column chromatography, eluting with a mixture of ethyl acetate and hexane (1:1) to afford brown powder (C1); yield: 1.89 g, 88%.

Spectral data of C1: ¹H NMR (400 MHz, CDCl₃): δ 7.77 (dd, J=4.9 Hz, 1.4 Hz, 2H), 7.37 (s, 1H), 6.97˜6.95 (m, 4H), 6.88 (dd, J=7.8 Hz, 4.9 Hz, 2H), 6.33 (s, 2H), 3.60˜3.51 (m, 2H), 3.28 (br, 2H), 3.58˜3.52 (m, 2H), 1.21 (d, J=6.2 Hz, 12H). ¹⁹F NMR (376 MHz, DMSO-d₆): δ −62.85 (s, 3 F).

### (v) Synthesis of hexafluorophosphate salt of 3,3′-(5-(trifluoromethyl)-1,3-phenylene)bis-(1-isopropyl-3H-imidazo[4,5-b]pyridin-1-ium) (D1)

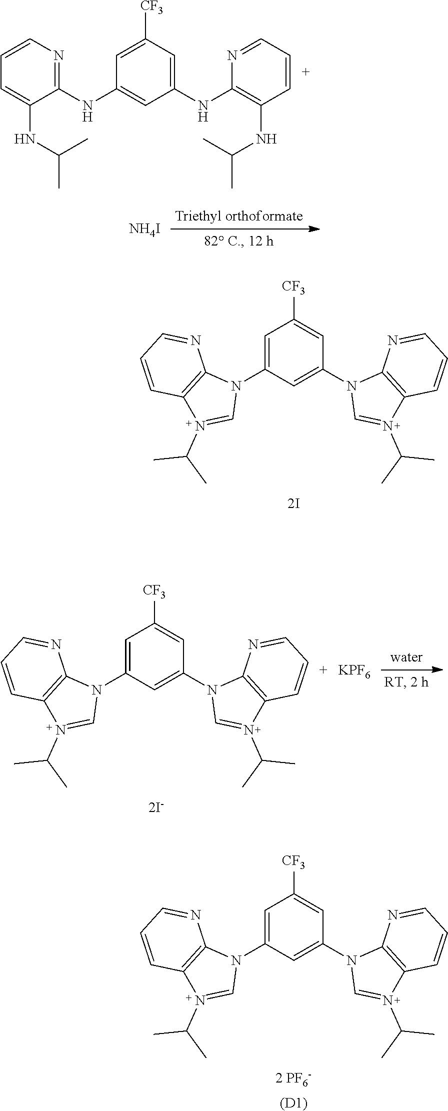

A mixture of NH₄I (1.33 g, 9.17 mmol), N²,N^{2′}-(5-(trifluoromethyl)-1,3-phenylene) bis(N³-isopropylpyridine-2,3-Diamine) (C) (1.63 g, 3.67 mmol) in triethyl orthoformate (20 mL) was heated at 82° C. for 12 hours. After cooled to RT, the resulting brown solid was filtered, washed with diethyl ether three times and dried in vacuo. It was next dissolved in ethanol. The white precipitation was immediately formed upon addition of an aqueous solution of KPF₆ (6.75 g, 36.7 mmol). It was collected by filtration, washed with diethyl ether (20 mL) and evaporated to dryness under vacuum to afford colorless solid (D1); yield: 2.41 g, 87%.

Spectral data of D1: ¹H NMR (400 MHz, acetone-d₆): δ 10.49 (s, 2H), 9.19˜9.18 (m, 1H), 8.91 (dd, J=4.8 Hz, 1.4 Hz, 2H), 8.88 (dd, J=8.5 Hz, 1.4 Hz, 2H), 8.75 (d, J=1.4 Hz, 2H), 7.98 (dd, J=4.8 Hz, 8.5 Hz, 2H), 5.51˜5.41 (m, 2H), 1.91 (d, J=6.8 Hz, 12H). ¹⁹F NMR (376 MHz, acetone-d₆): δ −63.26 (s, 3 F), −72.55 (d, J=711 Hz, 12 F). FD MS: m/z 611.2 (M-PF₆)⁺.

### Example 2

The adopted synthetic procedures for (D2) were depicted in Scheme 2.

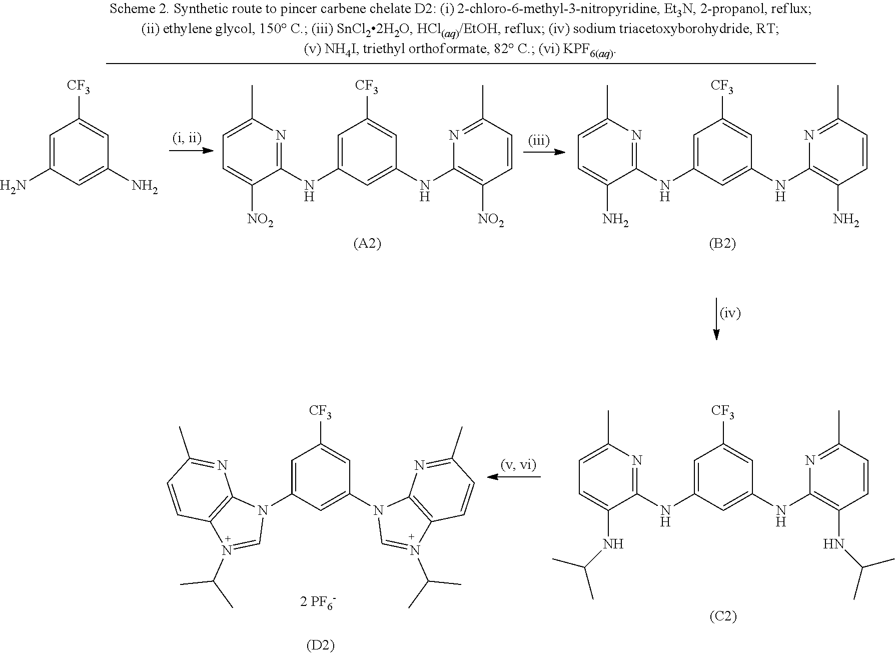

### N¹,N³-bis(6-methyl-3-nitropyridin-2-yl)-5-(trifluoromethyl)benzene-1,3-diamin (A2)

Except that 2-chloro-3-nitropyridine was replaced with 2-chloro-6-methyl-3-nitropyridine, the synthetic procedures of (A2) were similar to those of (A1); yield: 95%.

Spectral data of A2: brown solid; yield: 95%; ¹H NMR (500 MHz, CDCl₃): δ 10.39 (s, 2H), 8.95 (d, J=8.6 Hz, 2H), 8.26 (s, 1H), 7.99 (s, 2H), 6.78 (d, J=8.6 Hz, 2H), 2.57 (s, 6H). ⁹F NMR (470 MHz, acetone-d₆): δ −63.02 (s, 3 F).

### (iii) N²,N^{2′}-(5-(trifluoromethyl)-1,3-phenylene)bis(6-methylpyridine-2,3-diamine) (B2)

A mixture of N¹,N³-bis(6-methyl-3-nitropyridin-2-yl)-5-(trifluoromethyl) benzene-1,3-diamine (A2, 2.98 g, 6.65 mmol), Tin(II) chloride dehydrate (11.0 g, 53.2 mmol) in a mixture of HCl_{(aq)}/EtOH (2:3, 35 mL) were refluxed for 1 h. After cooled to RT, the mixture was quenched and extracted with ethyl acetate. The solution was neutralized with 1N NaOH_{(aq)} and washed with brine, dried over anhydrous Na₂SO₄ and concentrated to afford brown powder (B2); yield: 1.68 g, 65%.

Spectral data of B2: ¹H NMR (500 MHz, CDCl₃): δ 7.40 (s, 1H), 7.05 (s, 2H), 6.96 (d, J=7.7 Hz, 2H), 6.67 (d, J=7.7 Hz, 2H), 6.47 (br, 2H), 2.38 (s, 6H), 1.69 (br, 4H). ¹⁹F NMR (470 MHz, acetone-d₆): δ −63.01 (s, 3 F).

### N²,N^{2′}-(5-(trifluoromethyl)-1,3-phenylene)bis(N³-isopropyl-6-methylpyridine-2,3-diamine) (C2)

Except that (B2) was replaced with (B1), the synthetic procedures of (C2) were similar to those of (C1); yield: 64%.

Spectral data of C2: brown solid; ¹H NMR (500 MHz, CDCl₃): δ 7.44 (s, 1H), 7.04 (s, 2H), 6.95 (d, J=8.0 Hz, 2H), 6.73 (d, J=8.0 Hz, 2H), 3.51-3.46 (m, 2H), 2.40 (s, 6H), 1.72 (s, 4H), 1.17 (d, J=6.7 Hz, 12H). ¹⁹F NMR (470 MHz, acetone-d₆): δ −62.97 (s, 3 F).

### hexafluorophosphate salt of 3,3′-(5-(trifluoromethyl)-1,3-phenylene)bis(1-isopropyl-5-methyl-3H-imidazo[4,5-b]pyridin-1-ium) (D2)

Except that (C1) was replaced with (C2), the synthetic procedures of (D2) were similar to those of (C1); yield: 59%.

Spectral data of D2: yellow solid; ¹H NMR (500 MHz, acetone-d₆): δ 10.34 (s, 2H), 9.24 (s, 1H), 8.74-8.72 (m, 4H), 7.85 (d, J=8.6 Hz, 2H), 5.43-5.37 (m, 2H), 2.76 (s, 6H), 1.89 (d, J=6.7 Hz, 12H). ¹⁹F NMR (470 MHz, acetone-d₆): δ −63.28 (s, 3 F), −72.56 (d, J=708 Hz, 12 F). FD MS: m/z 639.2 (M-PF₆)⁺.

### Example 3

The adopted synthetic procedures for (D3) were depicted in Scheme 3.

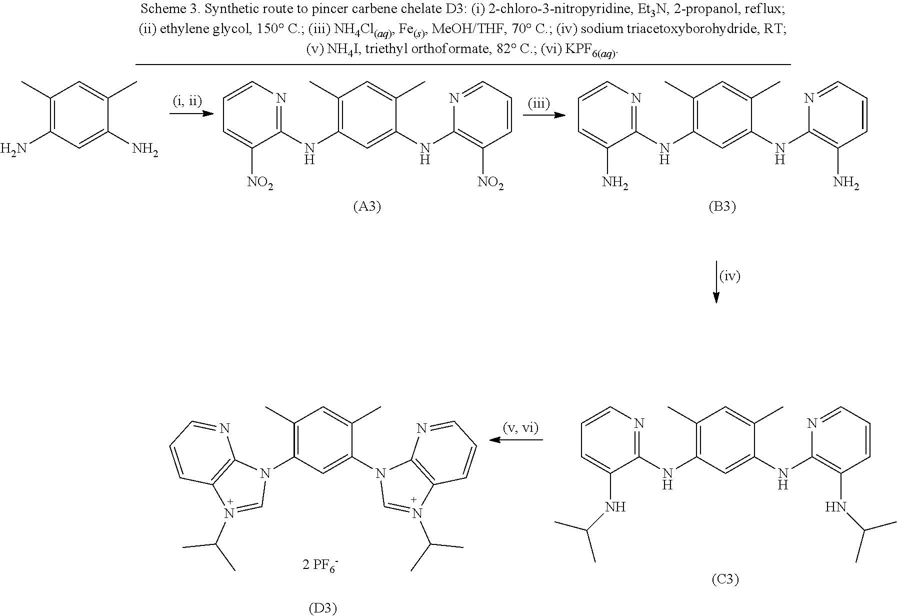

### 4,6-dimethyl-N¹,N³-bis(3-nitropyridin-2-yl)benzene-1,3-diamine (A3)

Except that 5-(trifluoromethyl)benzene-1,3-diamine was replaced with 3,5-dimethylpyridine-2,6-diamine, the synthetic procedures of (A3) were similar to those of (A1); yield: 86%.

Spectral data of A3: brown solid; ¹H NMR (400 MHz, CDCl₃): δ 9.89 (s, 2H), 8.52 (dd, J=8.4 Hz, 1.8 Hz, 2H), 8.44 (dd, J=4.6 Hz, 1.8 Hz, 2H), 8.20 (s, 1H), 7.21 (s, 1H), 6.79 (dd, J=8.4 Hz, 4.6 Hz, 2H), 2.31 (s, 6H).

### N,N^{2′}-(4,6-dimethyl-1,3-phenylene)bis(pyridine-2,3-diamine) (B3)

Except that (A1) was replaced with (A3), the synthetic procedures of (B3) were similar to those of (B1); yield: 81%.

Spectral data of B3: brown solid; ¹H NMR (400 MHz, CDCl₃): δ 7.72 (d, J=4.9 Hz, 2H), 7.10 (s, 1H), 7.01 (s, 1H), 6.94 (d, J=7.5 Hz, 2H), 6.69 (dd, J=7.5 Hz, 4.9 Hz, 2H), 5.93 (br, 2H), 3.48 (br, 2H), 2.19 (s, 6H), 1.73 (br, 2H).

### N²,N^{2′}-(4,6-dimethyl-1,3-phenylene)bis(N³-isopropylpyridine-2,3-diamine) (C3)

Except that (B1) was replaced with (B3), the synthetic procedures of (C3) were similar to those of (C1); yield: 61%.

Spectral data of C3: dark green solid; ¹H NMR (400 MHz, CDCl₃): δ 7.66 (dd, J=4.9 Hz, 1.4 Hz, 2H), 7.07 (s, 1H), 7.00 (s, 1H), 6.86 (dd, J=7.7 Hz, 1.4 Hz, 2H), 6.74 (dd, J=7.7 Hz, 4.9 Hz, 2H), 5.90 (br, 2H), 3.53-3.47 (m, 2H), 2.18 (s, 6H), 1.71 (br, 2H), 1.16 (d, J=6.2 Hz, 12H).

### hexafluorophosphate salt of 3,3′-(4,6-dimethyl-1,3-phenylene)bis(1-isopropyl-3H-imidazo[4,5-b]pyridin-1-ium) (D3)

Except that (C1) was replaced with (C3), the synthetic procedures of (D3) were similar to those of (C1); yield: 80%.

Spectral data of D3: light brown solid; ¹H NMR (500 MHz, acetone-d₆): δ 10.05 (s, 2H), 8.87 (dd, J=4.9 Hz, 1.1 Hz, 2H), 8.84 (dd, J=8.6 Hz, 1.1 Hz, 2H), 8.04 (s, 1H), 7.93 (dd, J 8.6 Hz, 4.9 Hz, 2H), 7.87 (s, 1H), 5.40-5.35 (m, 2H), 2.40 (s, 6H), 1.87 (d, J=6.7 Hz, 12H). ¹⁹F NMR (470 MHz, acetone-d₆): δ −72.39 (d, J=708 Hz, 12 F). FD MS: m/z 571.2 (M-PF₆).

### Example 4

The adopted synthetic procedures for (D4) were depicted in Scheme 4.

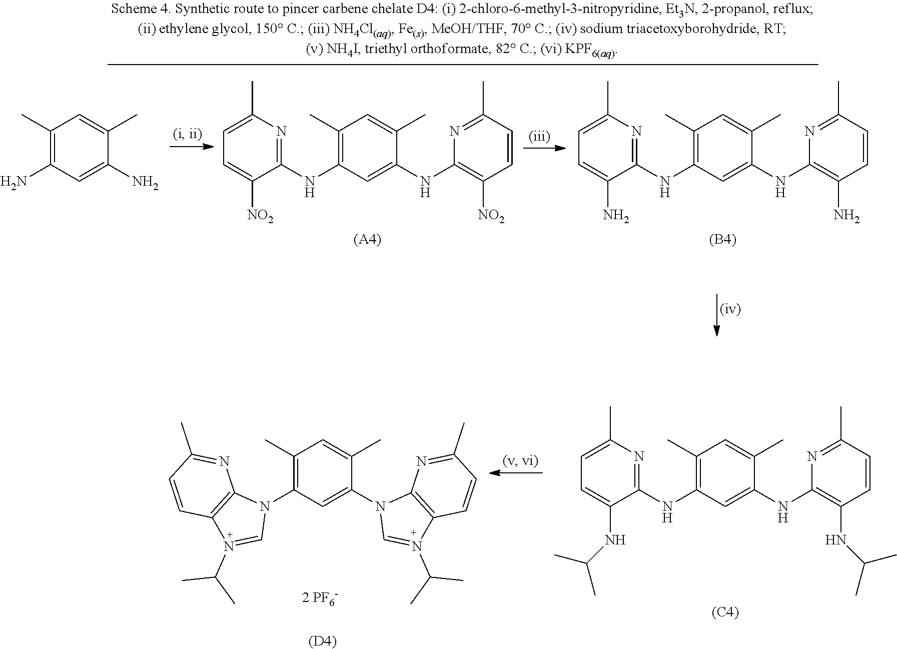

### 4,6-dimethyl-N¹,N³-bis(6-methyl-3-nitropyridin-2-yl)benzene-1,3-diamine (A4)

Except that 2-chloro-3-nitropyridine was replaced with 2-chloro-6-methyl-3-nitropyridine and 5-(trifluoromethyl)benzene-1,3-diamine was replaced with 3,5-dimethylpyridine-2,6-diamine, the synthetic procedures of (A4) were similar to those of (A1); yield: 95%.

Spectral data of A4: brown solid; ¹H NMR (400 MHz, CDCl₃): δ 10.06 (s, 2H), 8.55 (s, 1H), 8.40 (d, J=8.4 Hz, 2H), 7.16 (s, 1H), 6.63 (d, J=8.4 Hz, 2H), 2.42 (s, 6H), 2.32 (s, 6H).

### N²,N²′-(4,6-dimethyl-1,3-phenylene)bis(6-methylpyridine-2,3-diamine) (B4)

Except that (A1) was replaced with (A4), the synthetic procedures of (B4) were similar to those of (B1); yield: 90%.

Spectral data of B4: brown solid; ¹H NMR (400 MHz, CDCl₃): δ 6.95 (s, 1H), 6.89 (d, J=7.7 Hz, 2H), 6.59 (d, J=7.7 Hz, 2H), 5.97 (s, 1H), 3.31 (br, 2H), 2.31 (s, 6H), 2.23 (s, 6H), 1.68 (br, 4H).

### N²,N²′-(4,6-dimethyl-1,3-phenylene)bis(N³-isopropyl-6-methylpyridine-2,3-diamine) (C4)

Except that (B1) was replaced with (B4), the synthetic procedures of (C4) were similar to those of (C1); yield: 85%.

Spectral data of C4: orange solid; ¹H NMR (400 MHz, CDCl₃): δ 7.22 (s, 1H), 6.94 (s, 1H), 6.80 (d, J=7.8 Hz, 2H), 6.62 (d, J=7.8 Hz, 2H), 6.18 (br, 2H), 3.41-3.35 (m, 2H), 2.27 (s, 6H), 2.22 (s, 6H), 1.85 (br, 2H), 1.05 (d, J=6.2 Hz, 12H).

### hexafluorophosphate salt of 3,3′-(4,6-dimethyl-1,3-phenylene)bis(1-isopropyl-5-methyl-3H-imidazo[4,5-b]pyridin-1-ium) (D4)

Except that (C1) was replaced with (C4), the synthetic procedures of (D4) were similar to those of (D1); yield: 82%.

Spectral data of D4: light yellow solid; ¹H NMR (500 MHz, acetone-d₆): δ 9.88 (s, 2H), 8.68 (d, J=8.6 Hz, 2H), 8.03 (s, 1H), 7.84 (s, 1H), 7.80 (d, J=8.6 Hz, 2H), 5.34-5.29 (m, 2H), 2.72 (s, 6H), 2.38 (s, 6H), 1.85 (d, J=6.7 Hz, 12H). ¹⁹F NMR (470 MHz, acetone-d₆): δ −72.34 (d, J=708 Hz, 12 F). FD MS: m/z 599.2 (M-PF₆).

### Example 5

The adopted synthetic procedures for (D5) were depicted in Scheme 5.

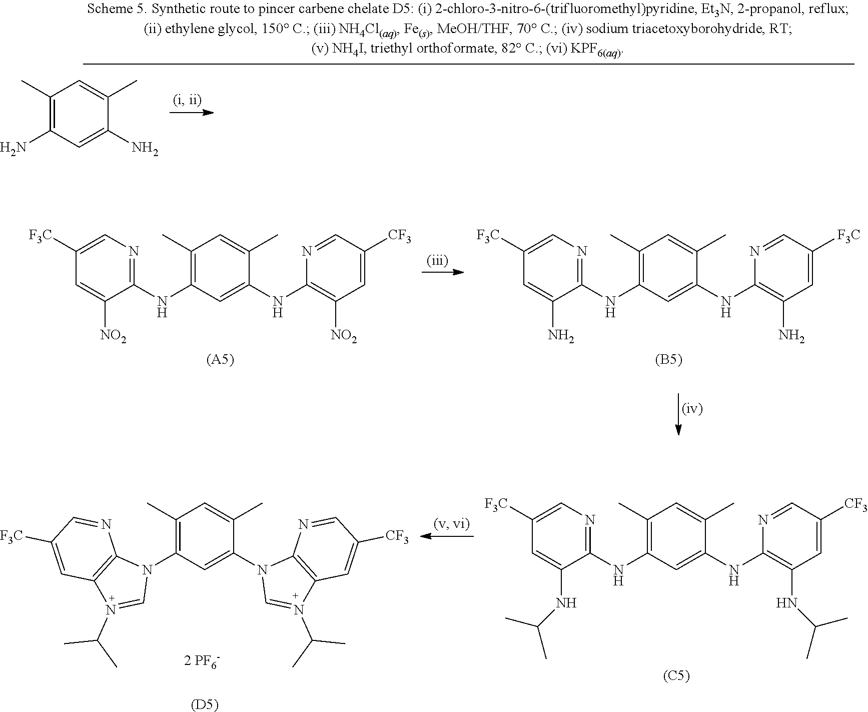

### 4,6-dimethyl-N¹,N³-bis(3-nitro-5-(trifluoromethyl)pyridin-2-yl)benzene-1,3-diamine (A5)

Except that 2-chloro-3-nitropyridine was replaced with 2-chloro-3-nitro-6-(trifluoromethyl)pyridine and 5-(trifluoromethyl)benzene-1,3-diamine was replaced with 3,5-dimethylpyridine-2,6-diamine, the synthetic procedures of (A5) were similar to those of (A1); yield: 98%.

Spectral data of A5: red solid; ¹H NMR (500 MHz, acetone-d₆): δ 10.16 (s, 2H), 8.80 (d, J=1.8 Hz, 2H), 8.72 (d, J=1.8 Hz, 2H), 8.10 (s, 1H), 7.30 (s, 1H), 2.33 (s, 6H). ¹⁹F NMR (470 MHz, acetone-d₆): δ −61.80 (s, 6 F).

### N²,N^{2′}-(4,6-dimethyl-1,3-phenylene)bis(5-(trifluoromethyl)pyridine-2,3-diamine) (B5)

Except that (A1) was replaced with (A5), the synthetic procedures of (B5) were similar to those of (B1); yield: 98%.

Spectral data of B5: brown solid; ¹H NMR (500 MHz, acetone-d₆): δ 7.79-7.78 (m, 3H), 7.19 (d, J=1.8 Hz, 2H), 7.10 (s, 2H), 7.07 (s, 1H), 4.86 (s, 4H), 2.20 (s, 6H). ¹⁹F NMR (470 MHz, acetone-d₆): δ −61.70 (s, 6 F).

### N²,N^{2′}-(4,6-dimethyl-1,3-phenylene)bis(N³-isopropyl-6-methylpyridine-2,3-diamine) (C5)

Except that (B1) was replaced with (B5), the synthetic procedures of (C5) were similar to those of (C1); yield: 59%.

Spectral data of C5: yellow solid; ¹H NMR (500 MHz, acetone-d₆): δ 7.74 (s, 2H), 7.57 (s, 1H), 7.20 (s, 2H), 7.06 (s, 1H), 6.99 (d, J=1.8 Hz, 2H), 4.55 (d, J=6.7 Hz, 2H), 3.79-3.73 (m, 2H), 2.16 (s, 6H), 1.28 (d, J=6.7 Hz, 12H). ¹⁹F NMR (470 MHz, acetone-d₆): δ −61.67 (s, 6 F).

### hexafluorophosphate salt of 3,3′-(4,6-dimethyl-1,3-phenylene)bis(1-isopropyl-6-(trifluoromethyl)-3H-imidazo[4,5-b]pyridin-1-ium) (D5)

Except that (C1) was replaced with (C5), the synthetic procedures of (D5) were similar to those of (C1); yield: 50%.

Spectral data of D5: light yellow solid; ¹H NMR (500 MHz, acetone-d₆): δ 10.25 (s, 2H), 9.35 (s, 2H), 9.23 (s, 2H), 8.09 (s, 1H), 7.91 (s, 1H), 5.55-5.49 (m, 2H), 2.43 (s, 6H), 1.90 (d, J=6.7 Hz, 12H). ¹⁹F NMR (470 MHz, acetone-d₆): δ −61.18 (s, 6 F), −72.30 (d, J=709 Hz, 12 F). FD MS: m/z 707.2 (M-PF₆)⁺.

### [Preparation of Iridium Complex]

### Example 6

In an example, the iridium complex in the invention may be prepared by the reaction:

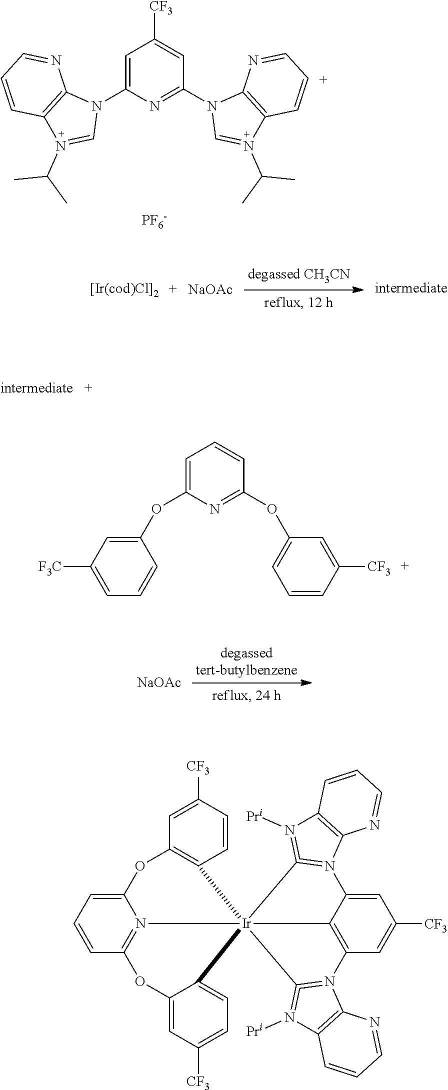

Preparation of Complex (IA-40):

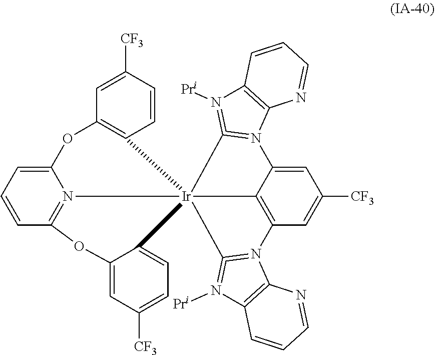

The functional di-imidazo[4,5-b]pyridin-2-ylidene chelate (1.0 mmol), [Ir(cod)(μ-Cl)]₂ (0.5 mmol) and NaOAc (5.0 mmol) were added in CH₃CN (80 mL) and the mixture was heated to reflux for 12 h. After removal of acetonitrile, the 2-pyrazolyl-6-phenyl pyridine class of chelate (1.0 mmol) and tert-butylbenzene (25 mL) were added, and the mixture was heated to reflux at 170° C. for another 24 h. After cooled to RT, tert-butylbenzene was evaporated in vacuo and the residue was dissolved in excess of ethyl acetate. This solution was washed with deionized water three times, dried over anhydrous Na₂SO₄ and concentrated to dryness. The crude product was purified by column chromatography, eluting with a 1:3 mixture of ethyl acetate and hexane to afford a light yellow bis-tridentate Ir(III) complex; yield: 8%.

Data of complex (IA-40): ¹H NMR (500 MHz, acetone-d₆): δ 8.86 (s, 2H), 8.55 (d, J=4.8 Hz, 2H), 8.41 (t, J=8.0 Hz, 1H), 8.19 (dd, J=8.0 Hz, 1.2 Hz, 2H), 7.62 (d, J=8.0 Hz, 4.8 Hz, 2H), 7.40 (dd, J=8.0 Hz, 4.8 Hz, 2H), 7.24 (d, J=1.2 Hz, 2H), 6.60 (dd, J=8.0 Hz, 1.2 Hz, 2H), 6.08 (d, J=8.0 Hz, 2H), 4.15˜4.10 (m, 2H), 1.17 (d, J=6.7 Hz, 12H). ¹⁹F NMR (470 MHz, acetone-d₆): δ −60.32 (s, 3 F), −62.27 (s, 6 F). FD MS: m/z 1053.1 (M)⁺. Anal. Calcd. For C₄₆H₃₃F₉IrN₅O₂: C, 50.19; H, 2.97; N, 9.31. Found: C, 50.12; H, 3.43; N, 9.23.

Selected crystal data of IA-40: C₃₈H₂₉F₉IrN₅O₂; M=950.86; monoclinic; space group=P2₁/n; a=11.3831(5) Å, b=21.3979(9) Å, c=15.0936(3) Å; β=106.6845(12°); V=3521.6(3) Å³; Z=4; ρ_{Calcd}=1.793 Mg·m⁻³; F(000)=1864, crystal size=0.202×0.195×0.060 mm³; λ(Mo−Kα)=0.71073 Å; T=150(2) K; μ=3.882 mm⁻¹; 28088 reflections collected, 10233 independent reflections (Rᵢₙₜ=0.0448), max. and min. transmission=0.7460 and 0.5466, data/restraints/parameters=10233/30/510, GOF=1.050, final R₁[I>2σ(I)]=0.0315 and wR₂ (all data)=0.0733.

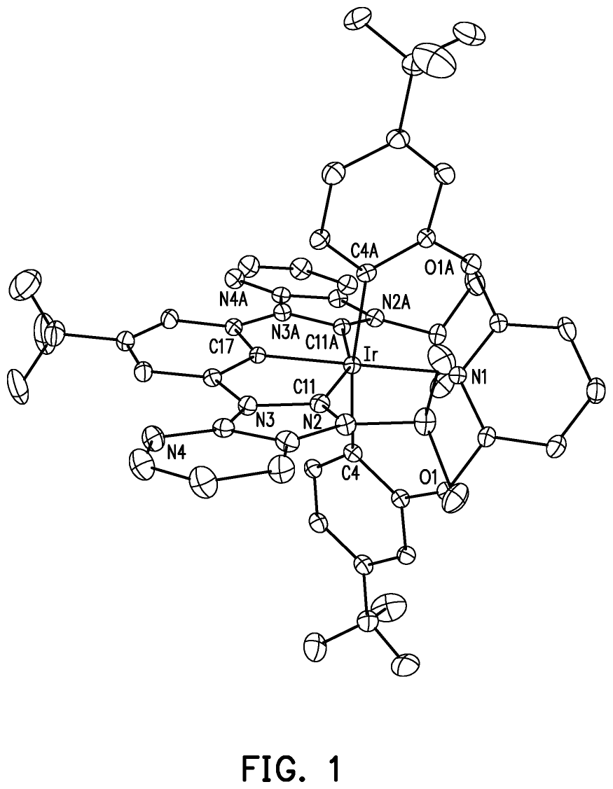

FIG. 1 depicts the single crystal X-ray diffraction structural diagram of complex (IA-40). The complex (IA-40) shown in FIG. 1 has two tridentate chelates residing on both sides of the central iridium metal to form an octahedral coordination structure. In particular, the partially interrupted conjugation on the left-side tridentate chelate forms a six membered coordination mode to the iridium metal, which is beneficial to reduce the angular strain upon coordination to the metal atom. This structural modification on chelate is expected to reduce the influence of structural distortion after the molecules are excited and to improve the emission quantum efficiency.

### Example 7

Preparation of Complex (IA-42):

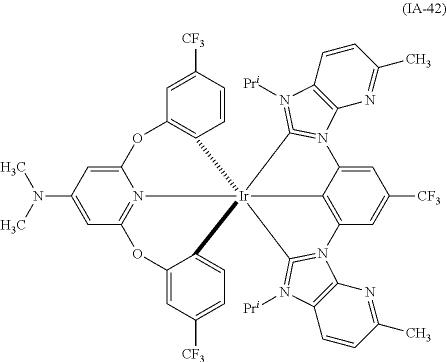

Except that D1 was replaced with D2 and 2,6-bis(3-(trifluoromethyl)phenoxy) pyridine was replaced with N,N-dimethyl-2,6-bis(3-(trifluoromethyl)phenoxy)pyridin-4-amine, the synthetic procedures of complex (IA-42) were similar to those of complex (IA-40); yield: 17%.

Data of complex (IA-42): ¹H NMR (500 MHz, acetone-d₆): δ 8.85 (s, 2H), 8.02 (d, J=8.6 Hz, 2H), 7.23 (d, J=8.6 Hz, 2H), 7.13 (s, 2H), 6.87 (s, 2H), 6.54 (d, J=7.3 Hz, 2H), 6.05 (d, J=7.3 Hz, 2H), 4.38-4.32 (m, 2H), 3.30 (s, 6H), 2.73 (s, 6H), 1.16 (d, J=6.7 Hz, 12H). ¹⁹F NMR (470 MHz, acetone-d₆): δ −60.21 (s, 3 F), −62.13 (s, 6 F). FD MS: m/z 1124.3 (M)⁺. Anal. Calcd. for C₄₈H₄₀F₉IrN₈O₂: C, 51.29; H, 3.59; N, 9.97. Found: C, 51.39; H, 3.67; N, 9.97.

### Example 8

Preparation of complex (IA-48):

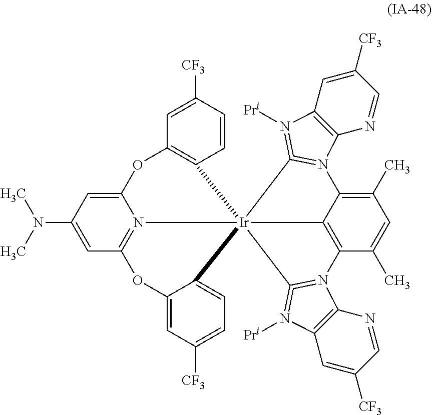

Except that D1 was replaced with D5 and 2,6-bis(3-(trifluoromethyl)phenoxy) pyridine was replaced with N,N-dimethyl-2,6-bis(3-(trifluoromethyl)phenoxy)pyridin-4-amine, the synthetic procedures of complex (IA-48) were similar to those of complex (IA-40); yield: 15%.

Data of complex (IA-48): ¹H NMR (500 MHz, acetone-d₆): δ 8.72 (d, J=1.2 Hz, 2H), 8.30 (d, J=1.2 Hz, 2H), 7.14 (d, J=1.2 Hz, 2H), 7.08 (s, 1H), 6.89 (s, 2H), 6.54 (dd, J=8.0 Hz, 1.2 Hz, 2H), 6.16 (d, J=8.0 Hz, 2H), 4.46-4.40 (m, 2H), 3.31 (s, 6H), 3.16 (s, 6H), 1.14 (d, J=7.4 Hz, 12H). ¹⁹F NMR (470 MHz, acetone-d₆): δ −60.79 (s, 6 F), −62.15 (s, 6 F). FD MS: m/z 1192.3 (M)⁺. Anal. Calcd. for C₄₉H₃₉F₁₂IrN₈O₂: C, 49.37; H, 3.30; N, 9.40. Found: C, 49.38; H, 3.74; N, 9.46.

### Example 9

Preparation of Complex (IA-52):

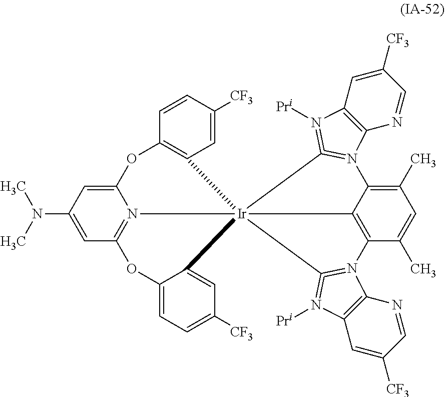

Except that D1 was replaced with D5 and 2,6-bis(3-(trifluoromethyl)phenoxy)pyridine was replaced with N,N-dimethyl-2,6-bis(4-(trifluoromethyl)phenoxy)pyridine-4-amine, the synthetic procedures of complex (IA-52) were similar to those of complex (IA-40); yield: 12%.

Data of complex (IA-52): ¹H NMR (500 MHz, acetone-d₆): δ 8.73 (d, J=1.2 Hz, 2H), 8.30 (d, J=1.2 Hz, 2H), 7.07 (s, 1H), 7.02 (d, J=8.0 Hz, 2H), 6.91 (dd, J=8.0 Hz, 1.8 Hz, 2H), 6.84 (s, 2H), 6.26 (d, J=1.8 Hz, 2H), 4.45-4.39 (m, 2H), 3.30 (s, 6H), 3.12 (s, 6H), 1.13 (d, J=6.7 Hz, 12H). ¹⁹F NMR (470 MHz, acetone-d₆): δ −60.77 (s, 6 F), −62.31 (s, 6 F). Anal. Calcd. for C₄₉H₃₉F₁₂IrN₈O₂: C, 49.37; H, 3.30; N, 9.40. Found: C, 49.69; H, 3.59; N, 9.24.

### Example 10

Preparation of Complex (IA-60):

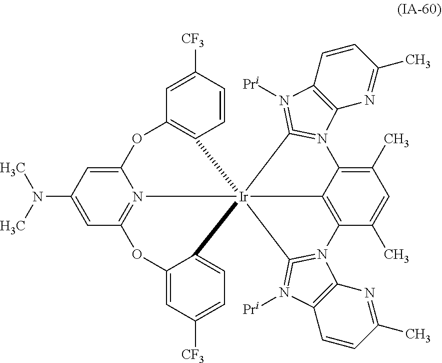

Except that D1 was replaced with D4 and 2,6-bis(3-(trifluoromethyl) phenoxy)pyridine was replaced with N,N-dimethyl-2,6-bis(3-(trifluoromethyl)phenoxy) pyridin-4-amine, the synthetic procedures of complex (IA-60) were similar to those of complex (IA-40); yield: 12%.

Data of complex (IA-60): ¹H NMR (500 MHz, acetone-d₆): δ 7.84 (d, J=8.0 Hz, 2H), 7.09 (d, J=4.3 Hz, 2H), 7.08 (d, J=2.4 Hz, 2H), 7.00 (s, 1H), 6.83 (s, 2H), 6.51 (d, J=8.0 Hz, 2H), 6.18 (d, J=8.0 Hz, 2H), 4.35-4.29 (m, 2H), 3.29 (s, 6H), 3.19 (s, 6H), 2.62 (s, 6H), 1.06 (d, J=6.7 Hz, 12H). ¹⁹F NMR (470 MHz, acetone-d₆): δ −62.00 (s, 3 F). FD MS: m/z 1084.3 (M)⁺. Anal. Calcd. for C₄₉H₄₅F₆IrN₈O₂: C, 54.28; H, 4.18; N, 10.34. Found: C, 54.31; H, 4.12; N, 10.37.

### Example 11

Preparation of Complex (IA-64):

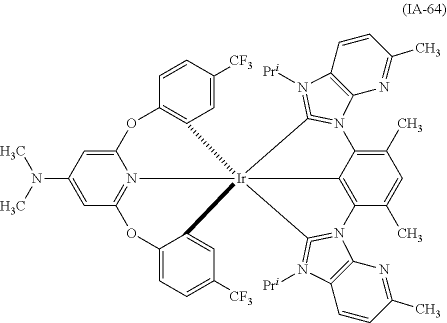

Except that D1 was replaced with D4 and 2,6-bis(3-(trifluoromethyl)phenoxy)pyridine was replaced with N,N-dimethyl-2,6-bis(4-(trifluoromethyl)phenoxy)pyridine-4-amine, the synthetic procedures of complex (IA-64) were similar to those of complex (IA-40); yield: 13%.

Data of complex (IA-64): ¹H NMR (500 MHz, acetone-d₆): δ 7.84 (d, J=8.0 Hz, 2H), 7.09 (d, J=4.3 Hz, 2H), 7.08 (d, J=2.4 Hz, 2H), 7.00 (s, 1H), 6.83 (s, 2H), 6.51 (d, J=8.0 Hz, 2H), 6.18 (d, J=8.0 Hz, 2H), 4.35-4.29 (m, 2H), 3.29 (s, 6H), 3.19 (s, 6H), 2.62 (s, 6H), 1.06 (d, J=6.7 Hz, 12H). ¹⁹F NMR (470 MHz, acetone-d₆): δ −62.00 (s, 3 F). FD MS: m/z 1084.3 (M)⁺. Anal. Calcd. for C₄₉H₄₅F₆IrN₈O₂: C, 54.28; H, 4.18; N, 10.34. Found: C, 54.31; H, 4.12; N, 10.37.

### Example 12

Preparation of Complex (IA-74):

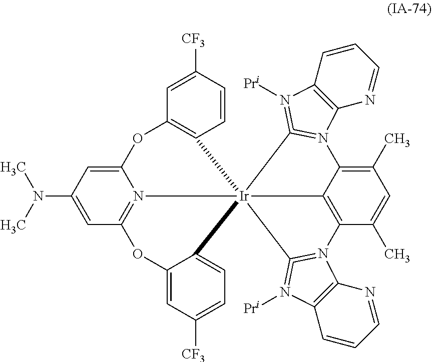

Except that D1 was replaced with D3 and 2,6-bis(3-(trifluoromethyl) phenoxy)pyridine was replaced with N,N-dimethyl-2,6-bis(3-(trifluoromethyl)phenoxy) pyridin-4-amine, the procedures to complex (IA-74) were similar to those of complex (IA-40); yield: 9%.

Data of complex (IA-74): ¹H NMR (500 MHz, DMSO-d₆): δ 8.37 (d, J=4.3 Hz, 2H), 8.09 (d, J=8.0 Hz, 2H), 7.27 (t, J=4.3 Hz, 2H), 7.13 (s, 2H), 6.97 (s, 1H), 6.82 (s, 2H), 6.56 (d, J=7.3 Hz, 2H), 6.01 (d, J=7.3 Hz, 2H), 4.22-4.16 (m, 2H), 3.19 (s, 6H), 3.12 (s, 6H), 0.98 (d, J=6.7 Hz, 12H). ¹⁹F NMR (470 MHz, DMSO-d₆): δ −60.09 (s, 3 F). FD MS: m/z 1056.4 (M)⁺. Anal. Calcd. for C₄₇H₄₁F₆IrN₈O₂: C, 53.45; H, 3.91; N, 10.61. Found: C, 53.37; H, 3.73; N, 10.62.

### Example 13

Preparation of Complex (IA-80):

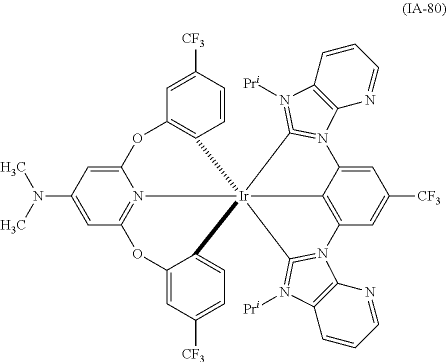

Except that 2,6-bis(3-(trifluoromethyl)phenoxy)pyridine was replaced with N,N-dimethyl-2,6-bis(3-(trifluoromethyl)phenoxy)pyridin-4-amine, the procedures to complex (IA-80) were similar to those of complex (IA-40); yield: 10%.

Data of complex (IA-80): ¹H NMR (500 MHz, DMSO-d₆, 353 K): δ 8.68 (s, 2H), 8.56 (d, J=4.9 Hz, 2H), 8.28 (d, J=8.0 Hz, 2H), 7.41 (dd, J=8.0 Hz, 4.9 Hz, 2H), 7.18 (d, J=1.2 Hz, 2H), 6.86 (s, 2H), 6.60 (d, J=8.0 Hz, 2H), 5.87 (d, J=8.0 Hz, 2H), 4.24˜4.19 (m, 2H), 3.21 (s, 6H), 1.08 (d, J=6.7 Hz, 12H). ¹⁹F NMR (470 MHz, DMSO-d₆, 353K): δ −58.32 (s, 3 F), −60.23 (s, 6 F). FD MS: m/z 1096.4 (M)⁺. Anal. Calcd. for C₄₈H₃₈F₉IrN₅O₂: C, 50.41; H, 3.31; N, 10.22. Found: C, 50.36; H, 3.55; N, 9.38.

### Example 14

In an example, the iridium complex in the invention may be prepared by the reaction:

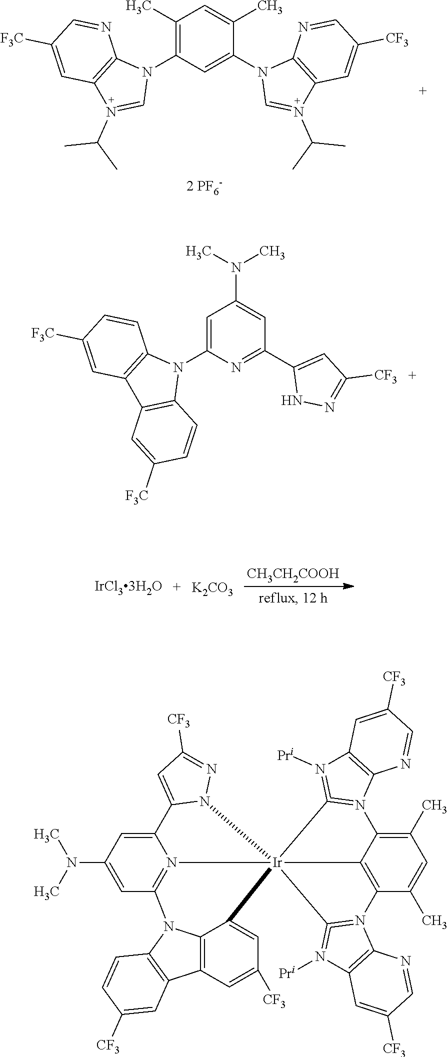

Preparation of complex (IA-130):

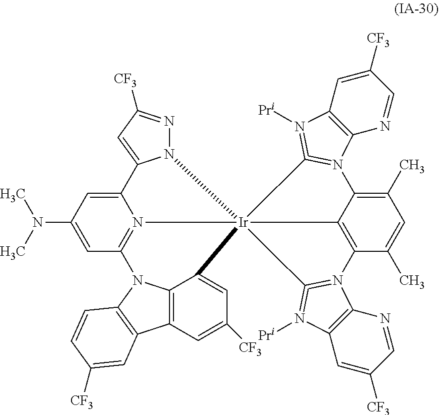

A mixture of D5 (345 mg, 0.37 mmol), 2-(3,6-bis(trifluoromethyl)-9H-carbazol-9-yl)-N,N-dimethyl-6-(3-(trifluoromethyl)-1H-pyrazol-5-yl)pyridin-4-amine (225 mg, 0.40 mmol), IrCl₃-3H₂O (143 mg, 0.41 mmol) and K₂CO₃ (1.12 g, 8.10 mmol) in propionic acid (30 mL) was refluxed under nitrogen for 12 h. After cooled to RT, propionic acid was evaporated in vacuo and the residue was dissolved in excess of ethyl acetate. This solution was washed with deionized water three times, dried over anhydrous Na₂SO₄ and concentrated to dryness. The crude product was purified by column chromatography, eluting with a mixture of ethyl acetate and hexane (1:5) to afford a white powder of IA-130 (101 mg, 20%).

Data of complex (IA-130): ¹H NMR (500 MHz, d₆-acetone): δ 8.76 (d, J=4.8 Hz, 1H), 8.73 (s, 2H), 8.60 (s, 1H), 8.24 (d, J=1.4 Hz, 2H), 7.88 (d, J=4.9 Hz, 2H), 7.64 (d, J=2.3 Hz, 1H), 7.55 (d, J=2.3 Hz, 1H), 7.17 (s, 1H), 7.12 (s, 1H), 6.37 (d, J=1.5 Hz, 1H), 4.66-4.56 (m, 2H), 3.45 (s, 6H), 3.15 (s, 6H), 1.19 (d, J=7.1 Hz, 6H), 0.89 (d, J=7.2 Hz, 6H). ¹⁹F NMR (470 MHz, d₆-acetone): δ −60.13 (s, 3F), −60.86 (s, 6F), −61.36 (s, 3F), −61.40 (s, 3F).

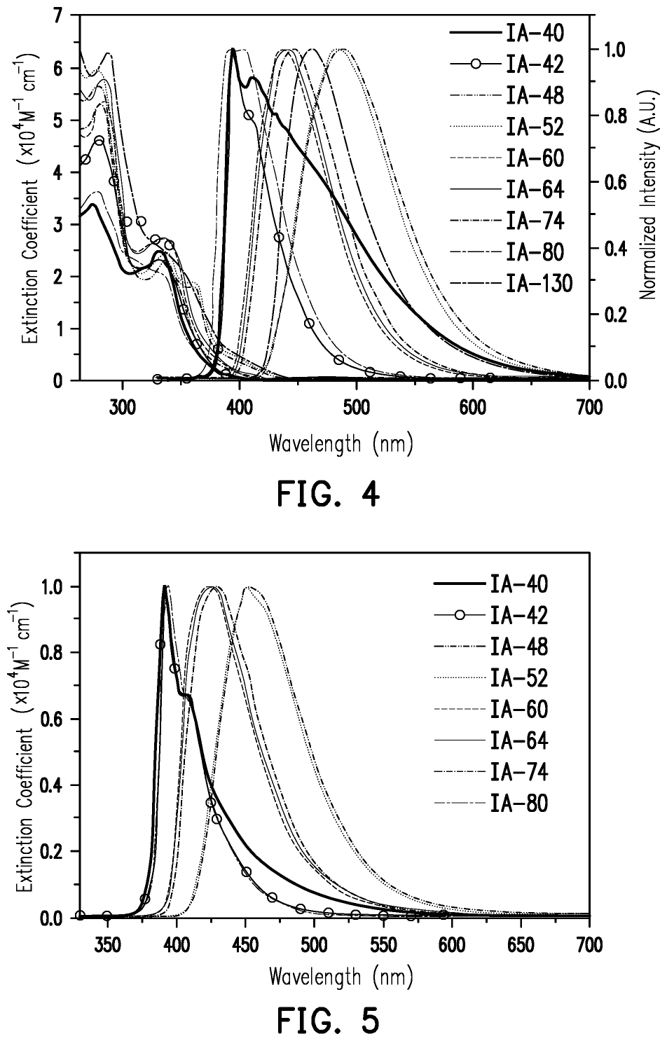

Both the absorption and emission spectra of complexes (IA-40), (IA-42), (IA-48), (IA-52), (IA-60), (IA-64), (IA-74), (IA-80), and (IA-130) mentioned in the invention were recorded methylene chloride at room temperature and are depicted in FIG. 4. The absorption peak positions (abs λₘₐₓ), emission peak positions (PL), quantum yields (Q.Y. %), observed lifetimes (τ_{obs}), radiation emission rate constants (kᵣ), non-radiation emission rate constants (kₙᵣ), and thermal decomposition temperatures (T_{d}) thereof are shown in Table 1. In particular, since the observed lifetime (τ_{obs}) is the reciprocal of the sum of the radiation emission rate constant (kᵣ) and the non-radiation emission rate constant (kₙᵣ), and the quantum yield is the ratio of the radiation rate constant to the sum of the radiation rate constant and the non-radiation rate constant, the radiation rate constant is (kᵣ=Q.Y. %/τ_{obs}); the non-radiation emission rate constant is

$$( {k}_{nr} = {k}_{r} ( \frac{1}{Q . Y . \%} - 1 ) ;$$

and the decomposition temperature is the temperature at which the complex loses 5% weight measured by heating the complex under nitrogen by 10° C. per minute while increasing the temperature continuously from 30° C. to 600° C.

TABLE 1

| | abs λₘₐₓ/nm | PL λₘₐₓ | P.L.Q.Y. | τ_{obs} | kᵣ | kₙᵣ | FWHM | T_{d} |
|---|---|---|---|---|---|---|---|---|
| | (ε × 10⁴ M⁻¹ cm⁻¹) ^{[a]} | (nm) ^{[a]} | (%) ^{[a,} ^{b]} | (μs) ^{[a]} | (10⁵ s⁻¹) | (10⁶ s⁻¹) | (cm⁻¹) ^{[c]} | (° C.) ^{[d]} |
| IA-40 | 275 (3.40), 333 (2.49) | 395, 412 (sh) | 1.1 | — | — | — | 5407 | 401 |
| IA-42 | 280 (4.61), 314 (3.07), 335 (2.76) | 439 | 6 | 0.20 | 3.1 | 4.8 | 2033 | 438 |
| IA-48 | 278 (5.67), 338 (2.31), 358 (1.81) | 488 | 33 | 0.34 | 9.8 | 2.0 | 3775 | 407 |
| IA-52 | 278 (5.98), 337 (2.48), 361 (1.91) | 487 | 57 | 0.52 | 11.0 | 0.8 | 3682 | 361 |
| IA-60 | 285 (5.38), 334 (2.71) | 449 | 16 | 0.19 | 8.3 | 4.3 | 2902 | 435 |
| IA-64 | 284 (5.80), 333 (2.76) | 450 | 9 | 0.10 | 9.0 | 9.1 | 2848 | 413 |
| IA-74 | 282 (5.30), 333 (2.32) | 455 | 39 | 0.40 | 9.7 | 1.5 | 3081 | 440 |
| IA-80 | 278 (3.62), 335 (2.03) | 402, 414 | 43.3 | 0.28 | 15.7 | 2.1 | 3320 | 423 |
| IA-130 | 254 (7.29), 287 (6.29), 349 (2.18) | 460 | 78 | 0.63 | 12.4 | 0.4 | 3271 | — |

^{[a]} UV-Vis spectra, PL spectra, lifetime and quantum yields were recorded in CH₂Cl₂ at a conc. of 10⁻⁵ M at RT.
^{[b]} Coumarin 102 (C102) in MeOH
(Q.Y. = 87% and λₘₐₓ = 480 nm) was employed as standard.
^{[c]} Full width at half maximum.
^{[d]} T_{d} stands for the temperature with 5% loss of weight.

The complexes (IA-40), (IA-42), (IA-48), (IA-52), (IA-60), (IA-64), (IA-74), and (IA-80) reported in the examples of the invention were dispersed in polymethylmethacrylate (PMMA) to afford thin film at concentration of 2 wt. %, to which the emission spectra thereof are shown in FIG. 5. The emission peak position (PL), the quantum yield (Q.Y. %), the observed lifetime (τ_{obs}), radiative lifetime (τ_{rad}), radiation emission rate constant (kᵣ), and non-radiation emission rate constant (kₙᵣ) are listed in Table 2. Herein, the radiative lifetime can be calculated from the observed lifetime divided by the emission quantum yield according to the equation (τ_{rad}=τ_{obs}/Q.Y.). Both the emission radiative rate constant (kᵣ) and non-radiative rate constant (kₙᵣ) are the reciprocal of observed lifetime (T_{obs}), radiative lifetime (τ_{rad}) and can be calculated from the photophysical data recorded, i.e., τ_{obs} and Q.Y. Therefore, the radiative rate constant is (kᵣ=Q.Y. %/τ_{obs}), and the non-radiative rate constant is

$$( {k}_{nr} = {k}_{r} ( \frac{1}{Q . Y . \%} - 1 ) ) .$$

In the measurement of the thermal decomposition temperature, the compound is heated by 10° C. per minute in nitrogen, the temperature is continuously increased from 30° C. to 600° C., and the temperature at which the weight loss of complex reaches 5% is recorded.

TABLE 2

| | PL λₘₐₓ | P.L.Q.Y. | τ_{obs} | τ_{rad} | kᵣ | kₙᵣ | FWHM |
|---|---|---|---|---|---|---|---|
| | (nm) ^{[a]} | (%) ^{[a,} ^{b]} | (μs) ^{[a]} | (μs) ^{[a]} | (10⁵ s⁻¹) | (10⁶ s⁻¹) | (cm⁻¹) ^{[c]} |
| IA-40 | 391, 408 (sh) | 17.4 | 0.48 ^{[d]} | 2.76 | 3.6 | 1.7 | 2108 |
| IA-42 | 436 | 23 | 0.71 | 3.1 | 3.2 | 1.1 | 1854 |
| IA-48 | 461 | 76 | 0.63 | 0.8 | 12.0 | 0.4 | 2976 |
| IA-52 | 460 | 89 | 0.59 | 0.7 | 15.1 | 0.2 | 3088 |
| IA-60 | 441 | 53 | 0.59 | 1.1 | 9.0 | 0.8 | 2333 |
| IA-64 | 441 | 48 | 0.49 | 1.0 | 9.8 | 1.1 | 2367 |
| IA-74 | 442 | 62 | 0.59 | 1.0 | 10.5 | 0.6 | 2411 |
| IA-80 | 394, 409 (sh) | 37.6 | 0.39 ^{[d]} | 1.0 | 9.6 | 1.6 | 1916 |

^{[a]} PL spectra, lifetime, and quantum yield were recorded in PMMA thin film with 2 wt. % of sample at RT.
^{[b]} Quantum yield was measured by integrated sphere.
^{[c]} Full width at half maximum.
^{[d]} the average of observed lifetime (av. τ_{obs}).

The compounds (IA-40) and (IA-80) (i.e., iridium complex with imidazo[4,5-b]pyridin-2-ylidene chelate) provided by the disclosure are compared in reference to the previously disclosed blue phosphors (1) and (2) (i.e., iridium complex formed by bis-tridentate imidazol-2-ylidene chelate), and in this way, both the nature and position of the chromophoric chelate in the corresponding metal complex and its influences on luminescence may thus be explored. It may be seen that this new type of iridium complex has a LUMO mainly localized at the imidazo[4,5-b]pyridin-2-ylidene chelate. In this case, the excited electrons may not enter the di-negative charged tridentate chelate. Because the dimethylamino group increases the LUMO energy level of this di-negative charged tridentate chelate; hence, the excited electrons can only enter the empty π*-orbital of the imidazo[4,5-b]pyridin-2-ylidene chelate. In this way, the emission spectrum is blue-shifted and blue phosphorescence with both high color purity and high efficiency can be achieved. The blue-emitting OLED devices with favorable performance characteristics may thus be produced.

The structures of conventional complexes (1) and (2) and complexes (I-40) and (I-80) of the invention are shown as follows:

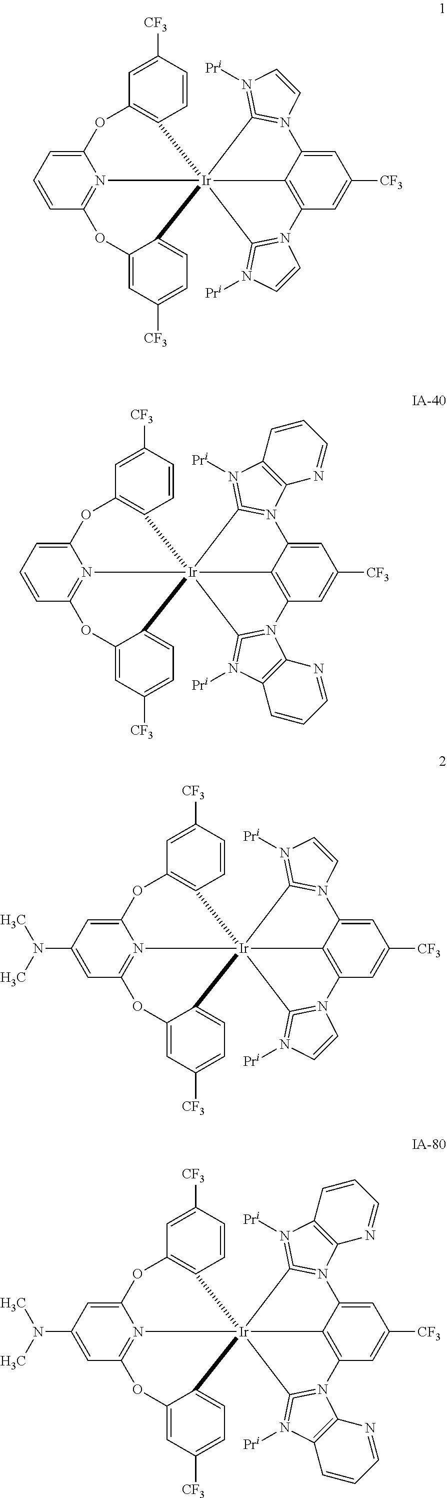

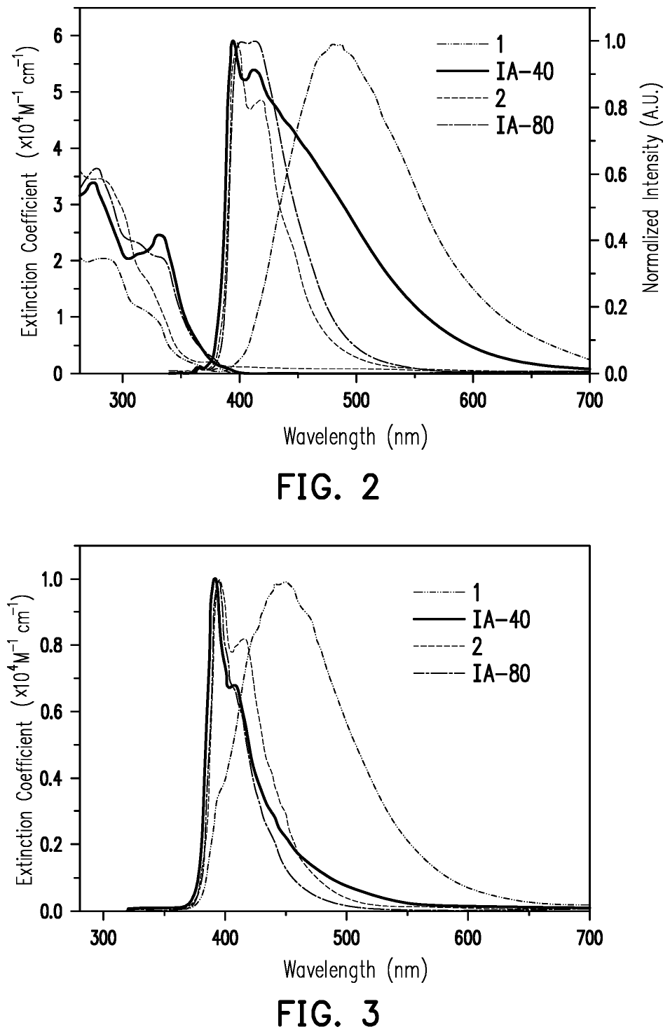

The absorption and emission spectrum of each of the complexes (1), (2), (IA-40), and (IA-80) are depicted in FIG. 2 and FIG. 3. Further, the absorption peak position (abs λₘₐₓ), emission peak position (PL), quantum yield (Q.Y. %), observed lifetime (τ_{obs}), thermal decomposition temperature (T_{d}), radiation rate constant (kᵣ), and non-radiation rate constant (kₙᵣ) are listed in Tables 3 and 4, respectively.

TABLE 3

| | abs λₘₐₓ/nm | PL λₘₐₓ | P.L.Q.Y. | τ_{obs} | kᵣ | kₙᵣ | FWHM | T_{d} |
|---|---|---|---|---|---|---|---|---|
| | (ε × 10⁴ M⁻¹ cm⁻¹) ^{[a]} | (nm) ^{[a]} | (%) ^{[a,} ^{b]} | (ns) ^{[a]} | (10⁵ s⁻¹) | (10⁶ s⁻¹) | (cm⁻¹) ^{[c]} | (° C.) ^{[d]} |
| 1 | 293 (1.98), 328 (0.98) | 482 | 4.7 | 573 | 0.8 | 1.7 | 5035 | 351 |
| IA-40 | 275 (3.40), 333 (2.49) | 395, 412 (sh) | 1.1 | — | — | — | 5407 | 401 |
| 2 | 278 (3.62), 335 (2.03) | 397, 418 (sh) | 5.9 | — | — | — | 2534 | 373 |
| IA-80 | 278 (3.62), 335 (2.03) | 402, 414 | 43.3 | 275 | 15.7 | 2.1 | 3320 | 423 |

^{[a]} UV-Vis spectra, PL spectra, lifetime and quantum yields were recorded in CH₂Cl₂ at a conc. of 10⁻⁵ M at RT.
^{[b]} Coumarin 102 (C102) in MeOH
(Q.Y. = 87% and λₘₐₓ = 480 nm) was employed as standard.
^{[c]} Full width at half maximum.
^{[d]} T_{d} stands for the temperature with 5% loss of weight.

TABLE 4

| | PL λₘₐₓ | P.L.Q.Y. | av. τ_{obs} | kᵣ | kₙᵣ | FWHM |
|---|---|---|---|---|---|---|
| | (nm) ^{[a]} | (%) ^{[a,} ^{b]} | (μs) ^{[a]} | (10⁵ s⁻¹) | (10⁶ s⁻¹) | (cm⁻¹) ^{[c]} |
| 1 | 450 | 24.7 | 6.85 | 0.36 | 0.1 | 4885 |
| IA-40 | 391, 408 (sh) | 17.4 | 0.48 ^{[d]} | 3.63 | 1.7 | 2108 |
| 2 | 394, 415 (sh) | 20.1 | 4.48 | 0.45 | 0.2 | 2692 |
| IA-80 | 394, 409 (sh) | 37.6 | 0.39 ^{[d]} | 9.64 | 1.6 | 1916 |

^{[a]} PL spectra, lifetime, and quantum yield were recorded in 2 wt. % doped PMMA thin film at RT.
^{[b]} Quantum yield was measured by integrated sphere.
^{[c]} Full width at half maximum.
^{[d]} kᵣ = Q.Y./τ_{obs} and kₙᵣ = (1 − Q.Y.)/τ_{obs}.

Therefore, in the disclosure, light-emitting efficiency is optimized, efficiency of the OLED device is enhanced, and the novel blue phosphorescent material is further developed. Since the imidazo[4,5-b]pyridin-2-ylidene chelate may provide the needed empty π*-orbital with lower energy and, hence, the corresponding LUMO energy level is also lower. Moreover, the dimethylamine in the bis-phenol-substituted pyridine chelate on (IA-80) increases the corresponding LUMO energy level of di-negative charged tridentate chelate. In this situation, the excited electrons may not enter the di-negative charged tridentate chelate, but may only enter the empty n-orbitals of the tridentate imidazo[4,5-b]pyridin-2-ylidene chelate. Eventually, the emission spectrum of this new complex is blue-shifted, and the emission lifetime is significantly shortened. The changes in emission properties all show that the complex is of great significance in the development of blue phosphorescent materials with high efficiency and adequate color purity.

In the structure of the new imidazo[4,5-b]pyridin-2-ylidene chelate, the nitrogen atom on the pyridine will induce certain instability, resulting in poor stability during sublimation. In order to address such problem, different substituents are placed on the benzene ring or pyridine respectively [complexes (IA-42), (IA-48), (IA-52), (IA-60), (IA-64), (IA-74), and (IA-130)] in order to protect the nitrogen atoms against the external perturbation, thereby effectively improved the thermal stability and enabling sublimation. In addition, by introducing electron donating groups (for example, methyl and other alkyl substituents) on the central benzene ring or inserting electron withdrawing groups (for example, trifluoromethyl and other perfluoroalkyl substituents) on the imidazo[4,5-b]pyridin-2-ylidene, the emission color may be adjusted to true blue with emission peak max. located at around 460 nm and, hence, the overall emission properties (quantum yield, emission lifetime, chromaticity etc.) may be optimized.

From FIGS. 4 and 5 and Tables 1 and 2, it may be seen that complexes (IA-42), (IA-48), (IA-52), (IA-60), (IA-64), (IA-74), and (IA-130) all have an emission peak max. located in the region between 390 nm and 480 nm, and have shorter photoluminescence lifetimes. Their emission properties show that they shall possess great significance in the development of blue phosphorescent materials with high efficiency and high color purity, and also have high potential for future optimization and production of OELD devices.

The iridium complex of the invention may be utilized in fabrication of OLED devices. These OLED devices include two electrodes, both the carrier transporting layers and a light-emitting layer disposed between the two electrodes, while the light-emitting layer includes at least one iridium complex of the invention. For example, the iridium complex of the invention is used as a dopant and incorporated into a host material of the light-emitting layer.

In an embodiment, the invention provides an OLED device including two electrodes and a light-emitting layer disposed between the two electrodes, wherein the light-emitting layer includes an iridium complex. The iridium complex may be used as a dopant to form the light-emitting layer. The material of each of the two electrodes may be selected from materials commonly used in the art, and other functional layers (such as electron-transport layer, hole-injection layer, hole-transport layer, or hole-blocking layer, etc.) may also be disposed between the electrode and light-emitting layer by techniques known in the art. The OLED device may be fabricated on planar surface or flexible substrates, such as conductive glass or plastic substrates.

Based on the above description, the invention is dedicated to the development of a high-efficiency phosphorescent material, particularly the blue emitting phosphorescent material, with both high efficiency and short photoluminescence lifetime. In order to meet these requirements, the structure is modified with a complex possessing two bis-tridentate chelate coordinated to the central iridium metal. The ππ* energy gap of carbene chelate is expected to be reduced by introducing the imidazo[4,5-b]pyridin-2-ylidene chelate, similar to the performance shown by the aforementioned carbene complex Ir(pmp)₃. Moreover, the ππ* energy gap and electron donating ability of the second, di-negative charged tridentate chelate are increased at the same time after incorporating the strong electron-donating group (NMe₂ and OMe, etc.) on its pyridine fragment, so as to enhance the MLCT contribution at the excited state, which is beneficial to the reduction of the radiation emission lifetime of the prepared iridium complex.

Moreover, in the invention, the issue that the imidazo[4,5-b]pyridin-2-ylidene complex itself is relatively unstable and may not be sublimated is also addressed. After introducing the alkyl or perfluoroalkyl substituents at the ortho-position of the pyridinyl appendage of imidazo[4,5-b]pyridin-2-ylidene, steric encumbrance may be introduced to the nitrogen atoms to reduce its reactivity against the external perturbation and improve the thermal stability of the prepared bis-tridentate iridium complex. Therefore, in the disclosure, light-emitting efficiency is optimized, operational lifetime of the OLED device is enhanced, and the novel blue phosphorescent material is further developed.

Although the invention has been described with reference to the above embodiments, it will be apparent to one having ordinary skill in the art that modifications to the described embodiments may be made without departing from the spirit of the invention. Accordingly, the scope of the invention is defined by the attached claims not by the above detailed descriptions.

What is claimed is:

1. An iridium complex, having a structure represented by general formula (I): 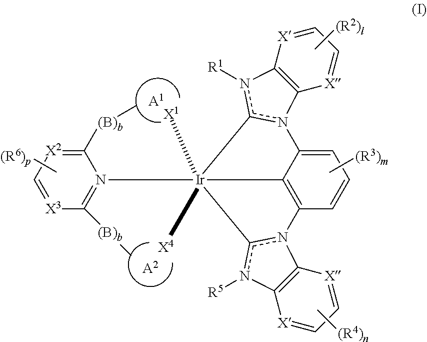
wherein
one of X′ and X″ is nitrogen and the other is carbon;
X¹, X², X³, and X⁴ are each independently carbon or nitrogen;
R¹ and R⁵ are each independently substituted or unsubstituted C₁-C₁₂ alkyl;
R², R³, and R⁴ are each independently hydrogen, substituted or unsubstituted C₁-C₁₂ alkyl, substituted or unsubstituted C₆-C₁₂ aryl, or —CₘF₂ₘ₊₁, and m is an integer of 1 to 3;
l, m, and n are each independently an integer of 1 to 3;
when l is equal to or greater than 2, two or more R²'s may be connected to each other to form a C₃-C₈ aromatic ring or a nitrogen-containing heteroaromatic ring;
when m is equal to or greater than 2, two or more R³'s may be connected to each other to form a C₃-C₈ aromatic ring or a nitrogen-containing heteroaromatic ring;
when n is equal to or greater than 2, two or more R⁴'s may be connected to each other to form a C₃-C₈ aromatic ring or a nitrogen-containing heteroaromatic ring;
A¹ and A² are each independently an unsaturated 5-membered ring or an unsaturated 6-membered ring;
B is —O—, —NR—, or —CR₂—, R is hydrogen or substituted or unsubstituted C₁-C₁₂ alkyl, and —NR— or —CR₂— may optionally be connected to A² to form a substituted or unsubstituted C₉-C₁₄ aromatic ring or a nitrogen-containing heteroaromatic ring;
b is 0 or 1;
R⁶ is hydrogen, fluorine, substituted or unsubstituted C₁-C₁₂ alkyl, substituted or unsubstituted C₁-C₆ alkoxy, substituted or unsubstituted C₆-C₁₂ aryl, substituted or unsubstituted C₁-C₆ amino, or —CₓF₂ₓ₊₁, and x is an integer of 1 to 3;
p is an integer of 1 to 3; and
when p is equal to or greater than 2, two or more R⁶'s may be connected to each other to form a C₃-C₈ aromatic ring or a nitrogen-containing heteroaromatic ring.

2. The iridium complex of claim 1, having a structure represented by general formula (IA): 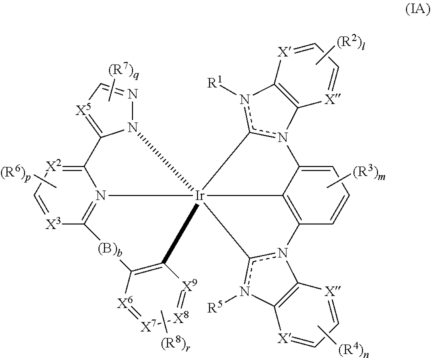
wherein
X⁵, X⁶, X⁷, X⁸, and X⁹ are each independently carbon or nitrogen;
R⁷ and R⁸ are each independently hydrogen, substituted or unsubstituted C₁-C₁₂ alkyl, substituted or unsubstituted C₁-C₆ alkoxy, substituted or unsubstituted C₆-C₁₂ aryl, or —CₓF₂ₓ₊₁, and x is an integer of 1 to 3;
q is an integer of 1 to 2;
r is an integer of 1 to 4;
when q is equal to or greater than 2, two or more R⁶'s may be connected to each other to form a C₃-C₈ aromatic ring or a nitrogen-containing heteroaromatic ring; and
when r is equal to or greater than 2, two or more R⁷'s may be connected to each other to form a C₃-C₈ aromatic ring or a nitrogen-containing heteroaromatic ring.

3. The iridium complex of claim 2, having a structure represented by any one of formula (IA-1) to formula (IA-144):

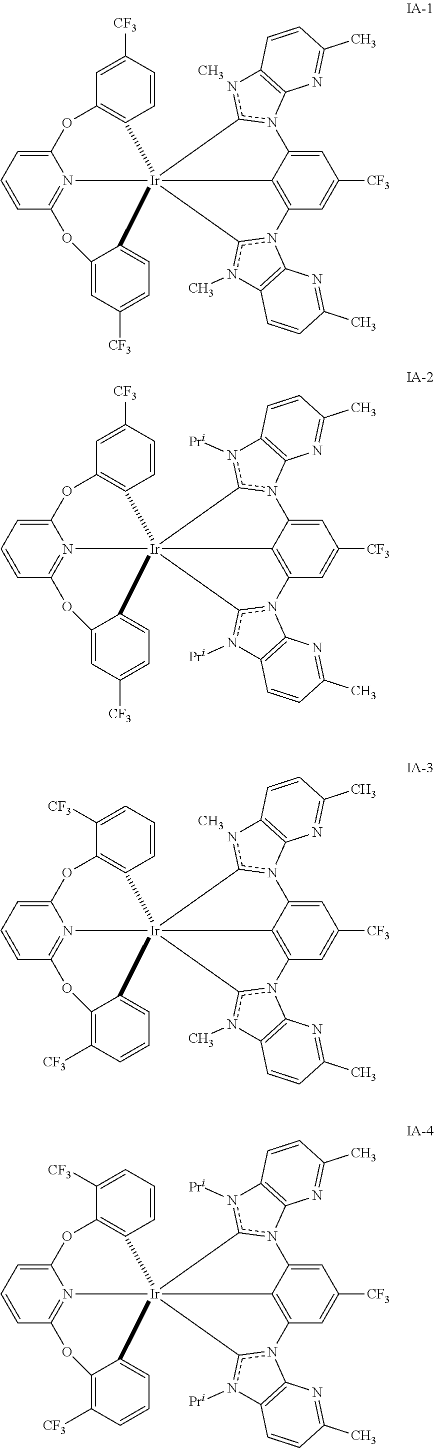

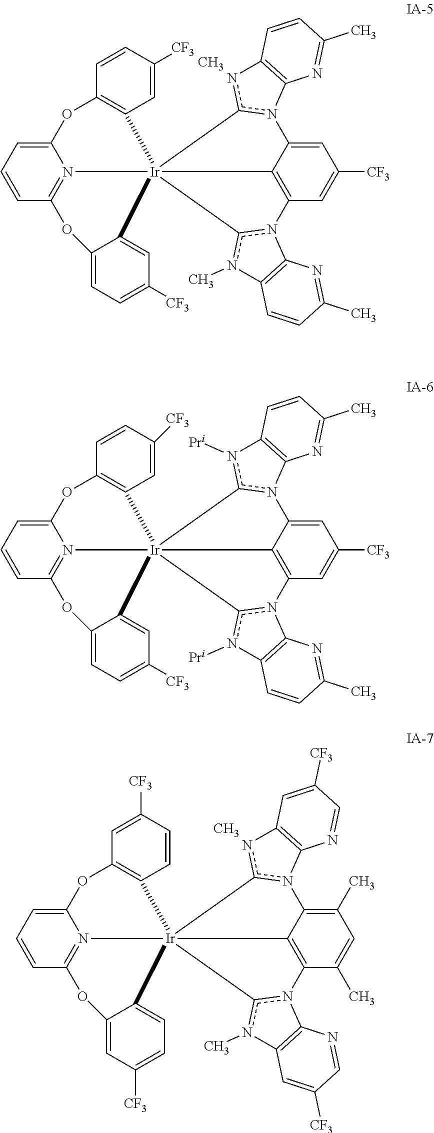

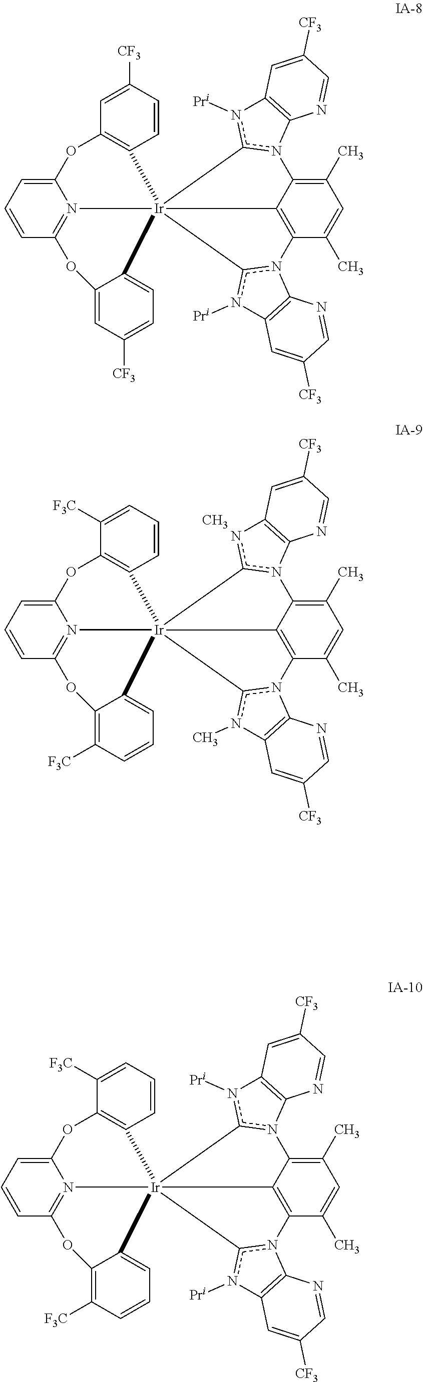

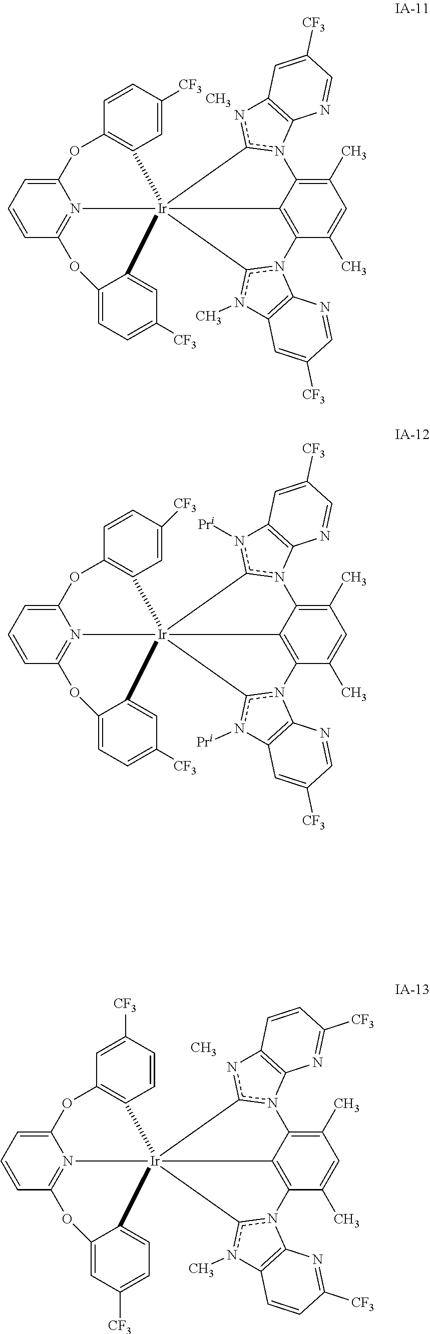

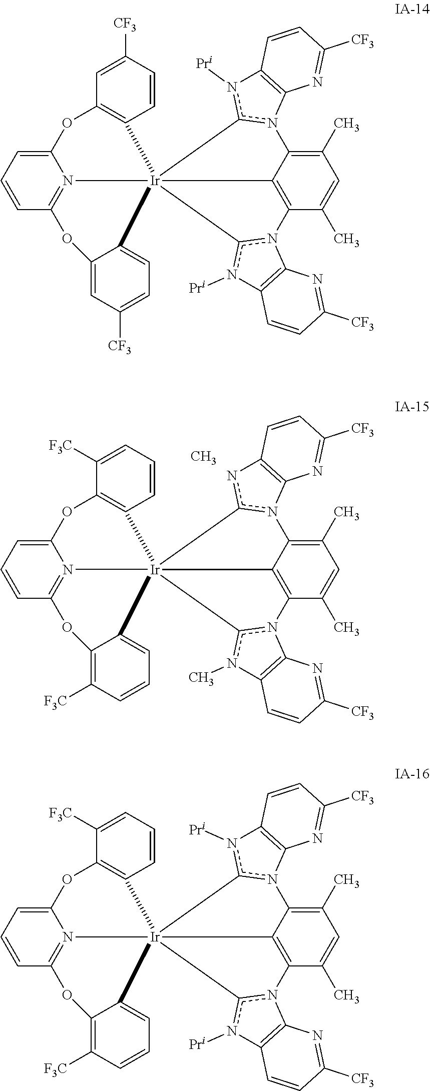

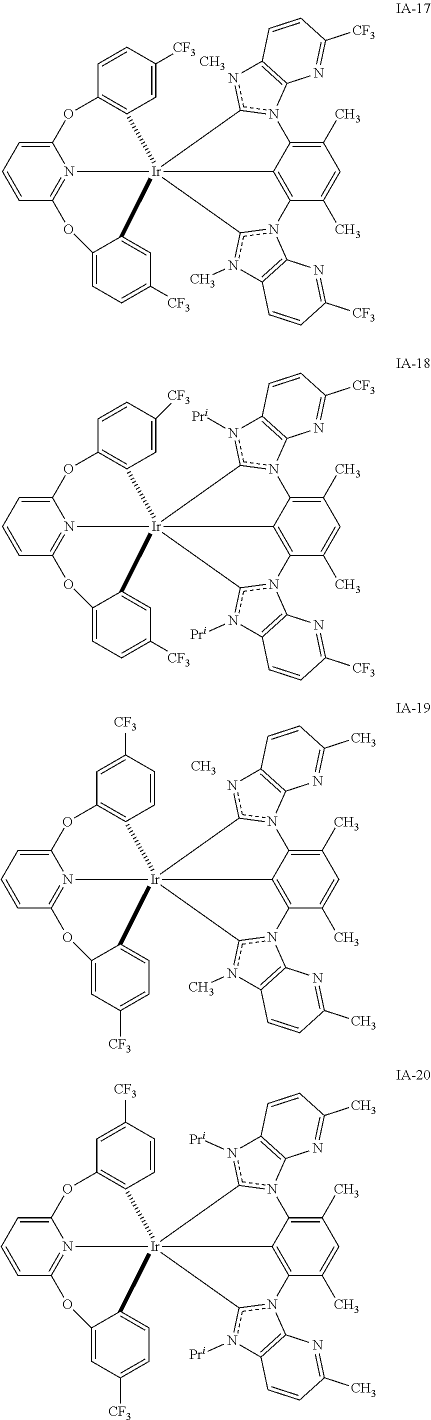

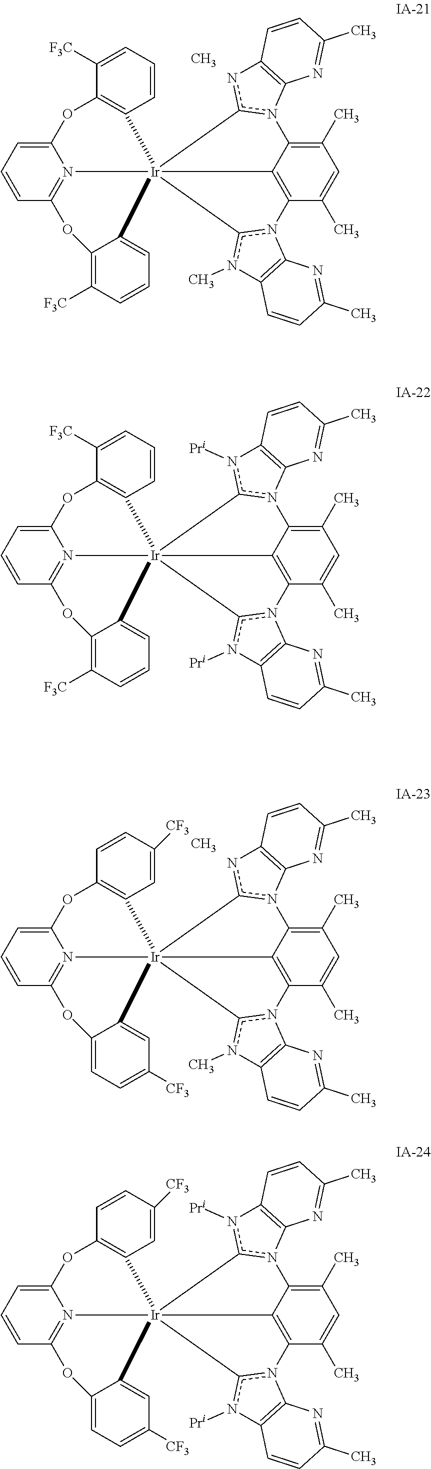

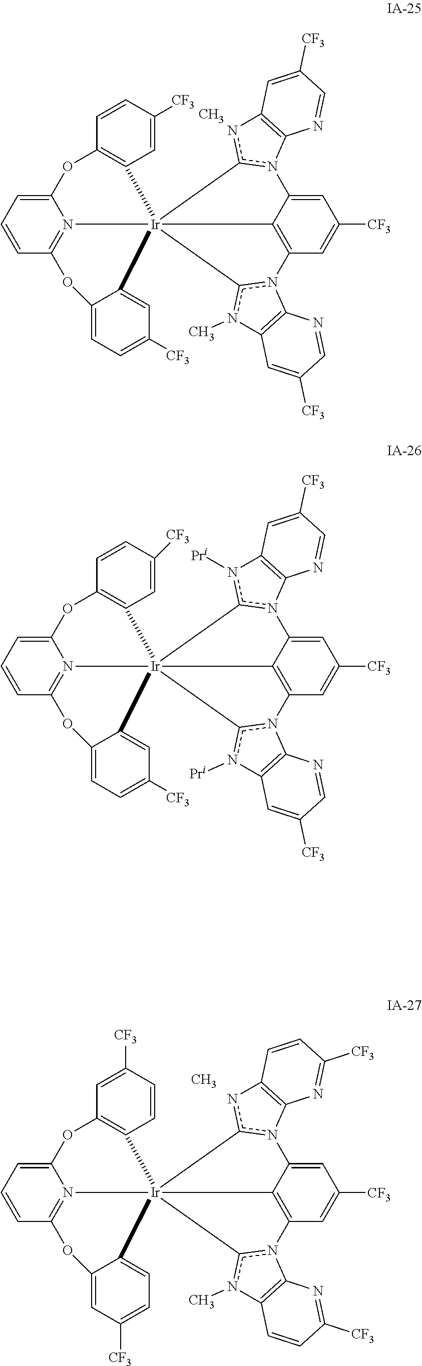

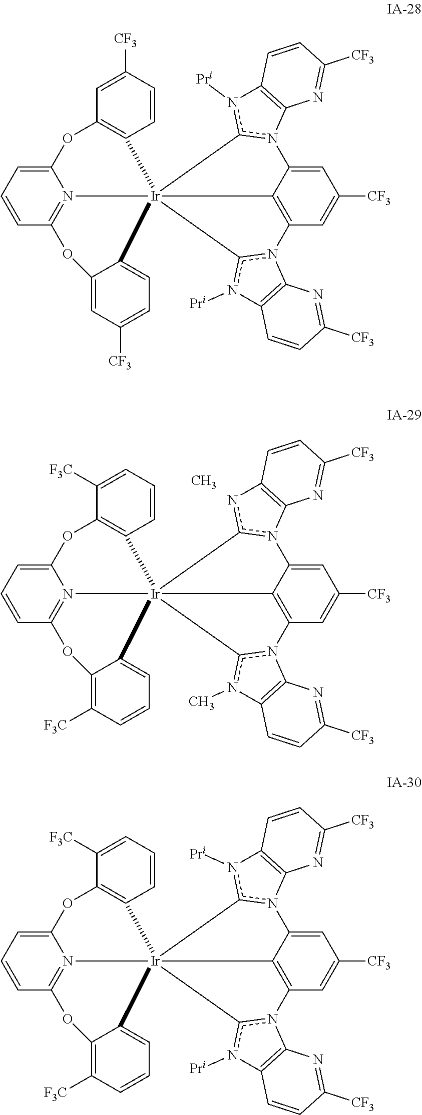

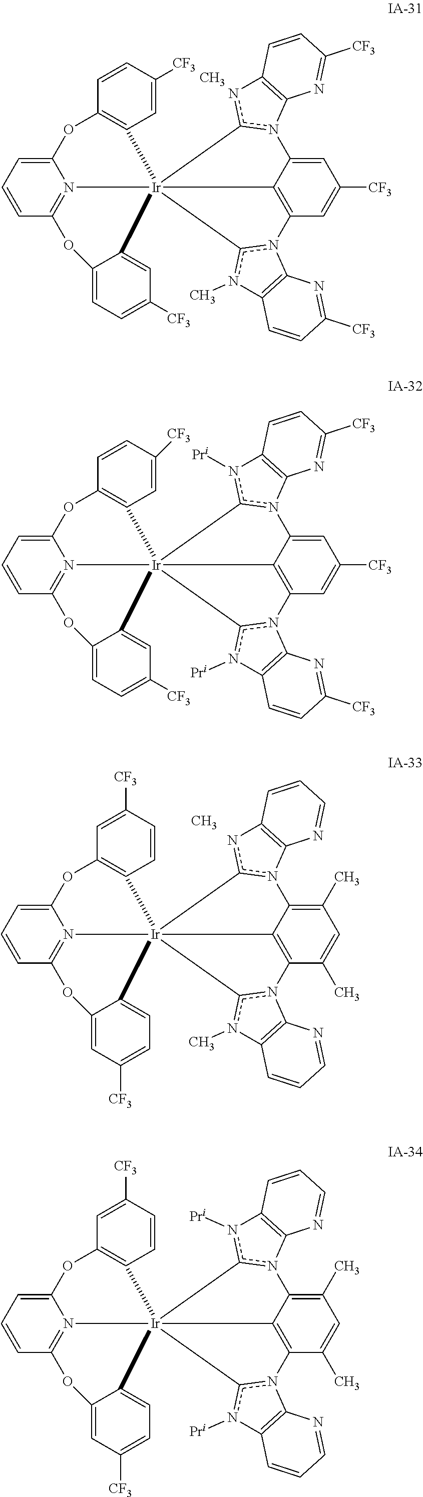

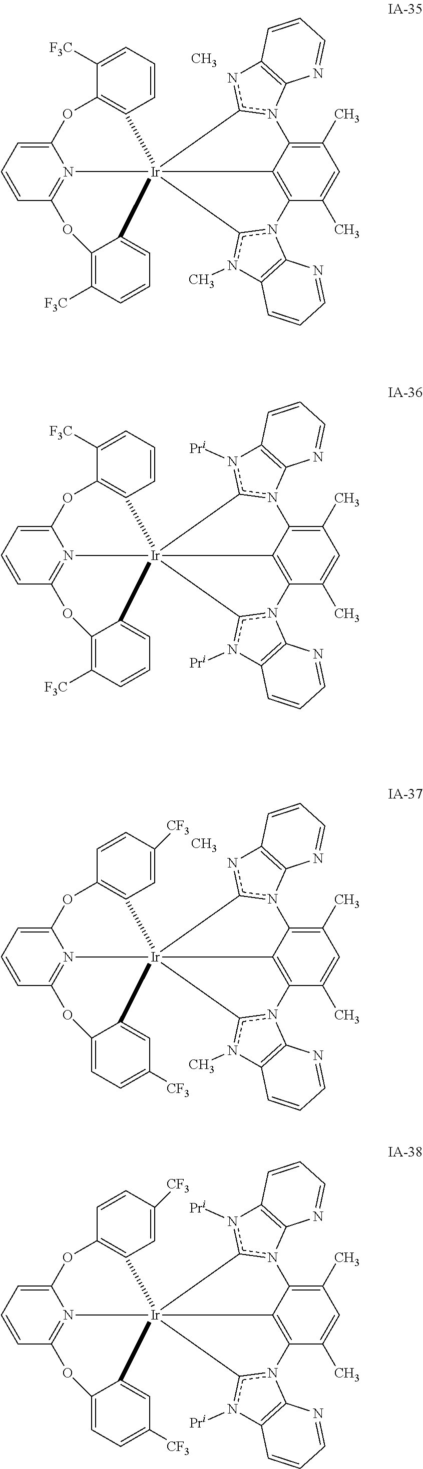

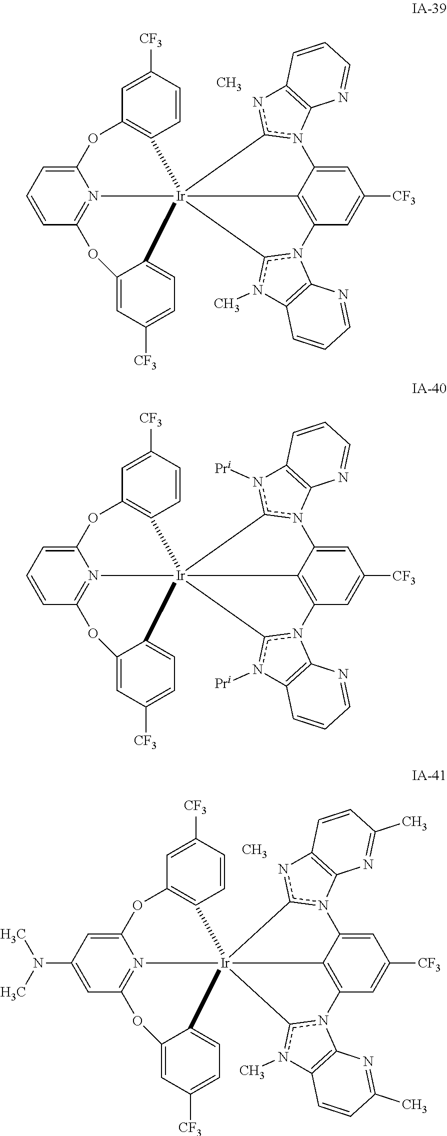

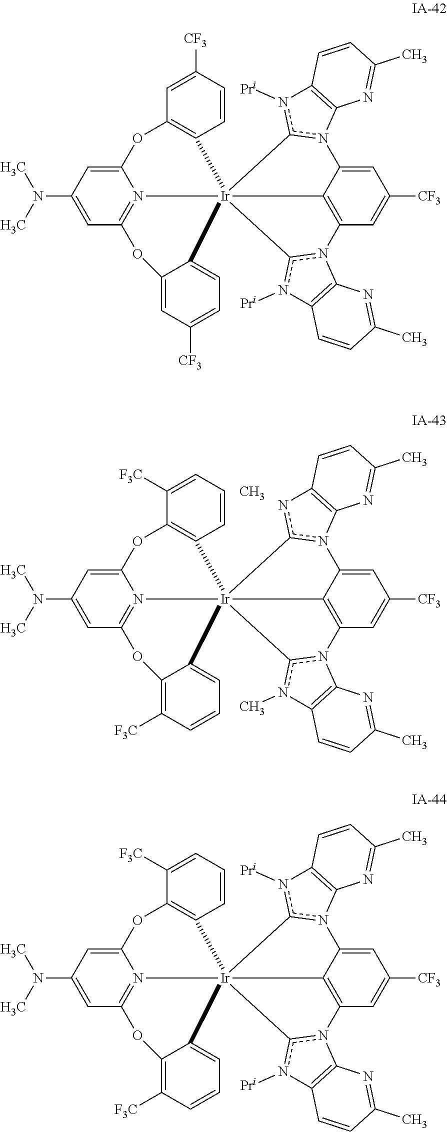

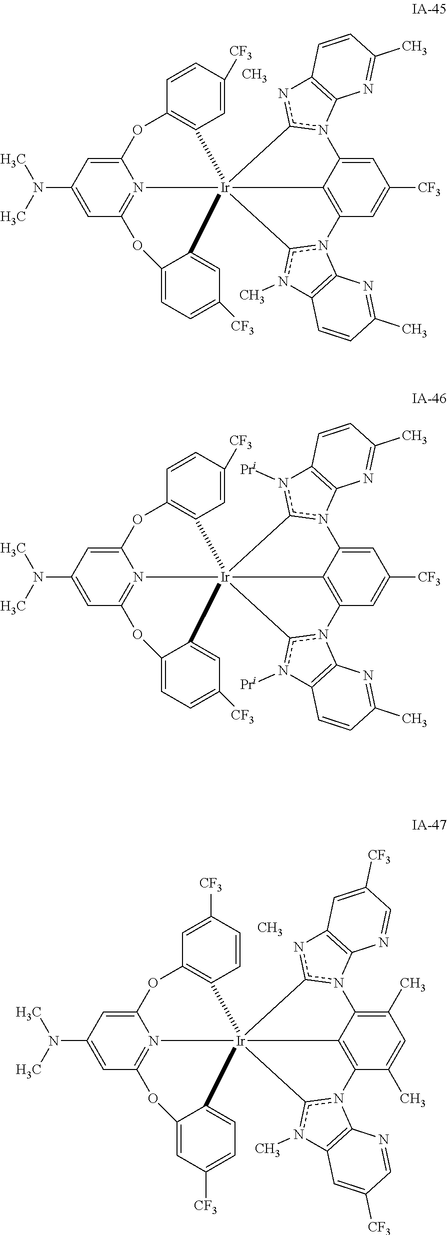

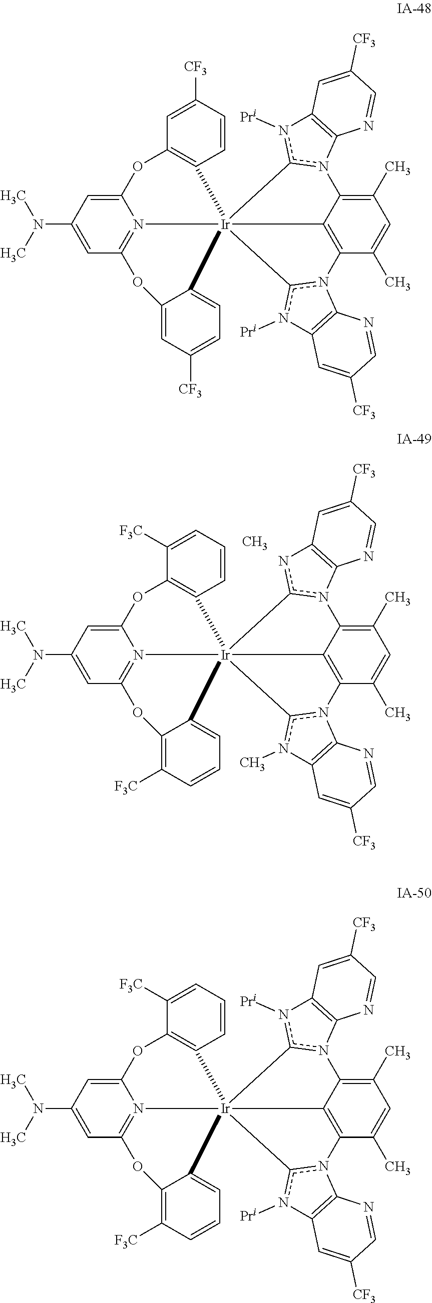

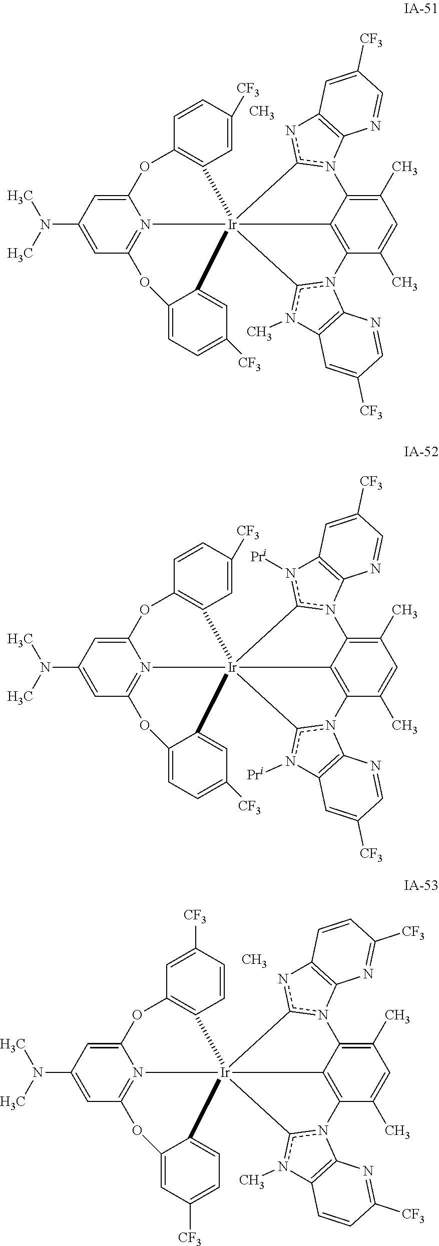

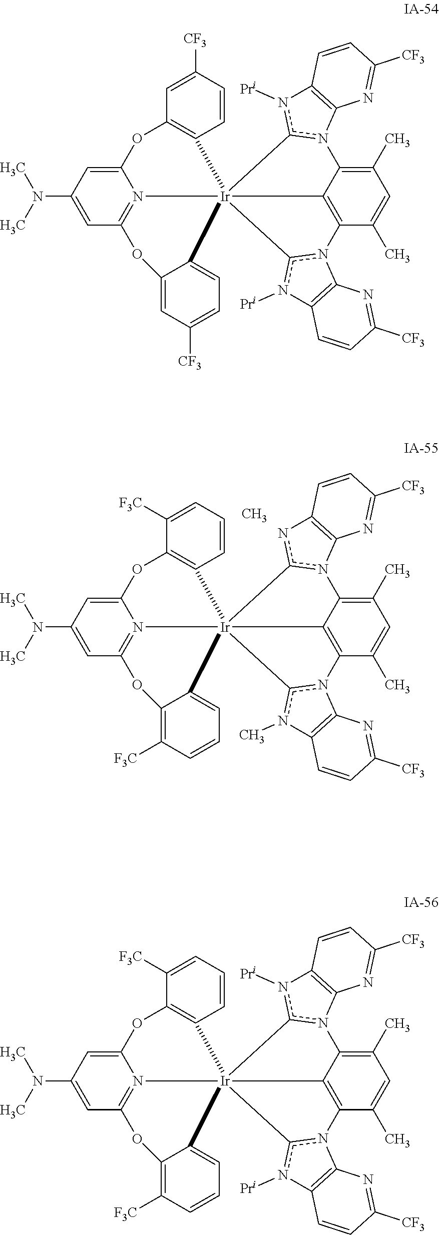

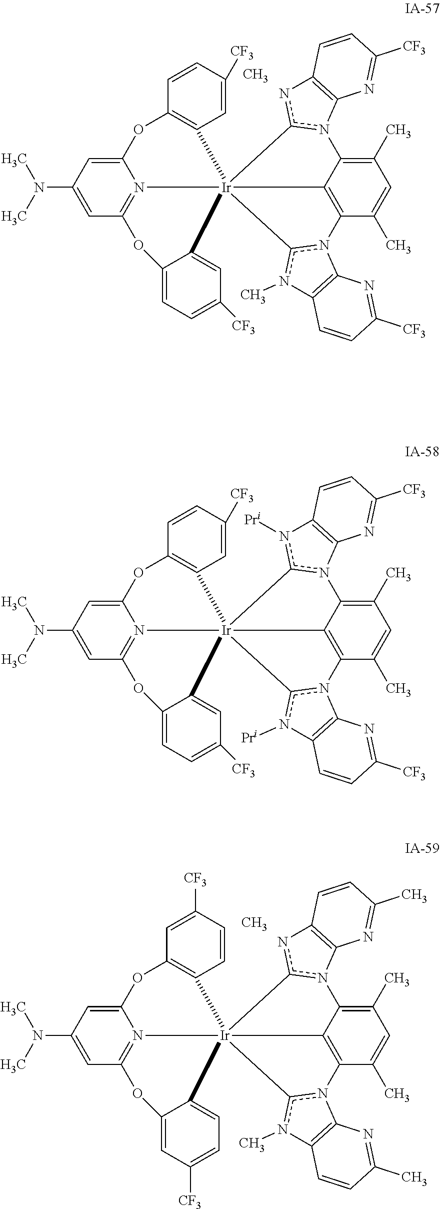

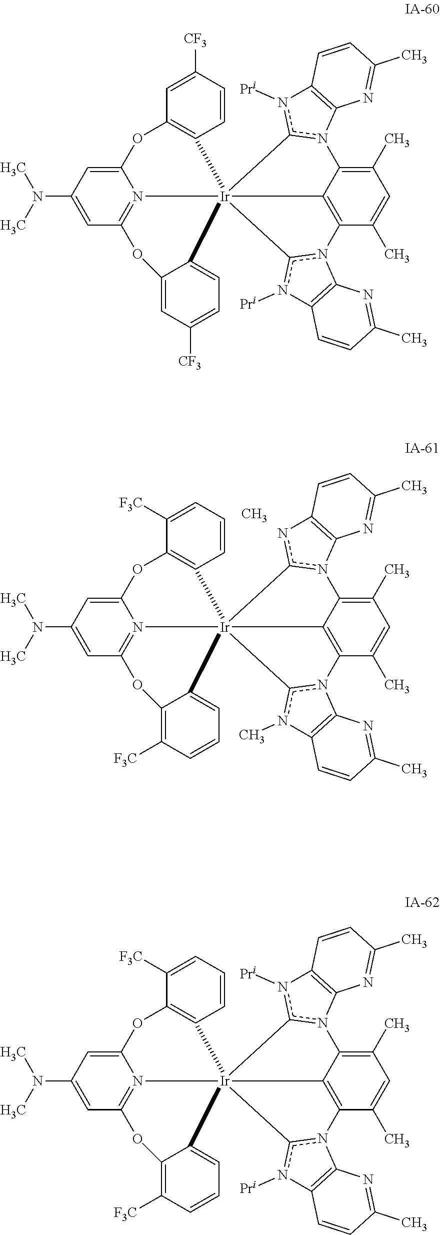

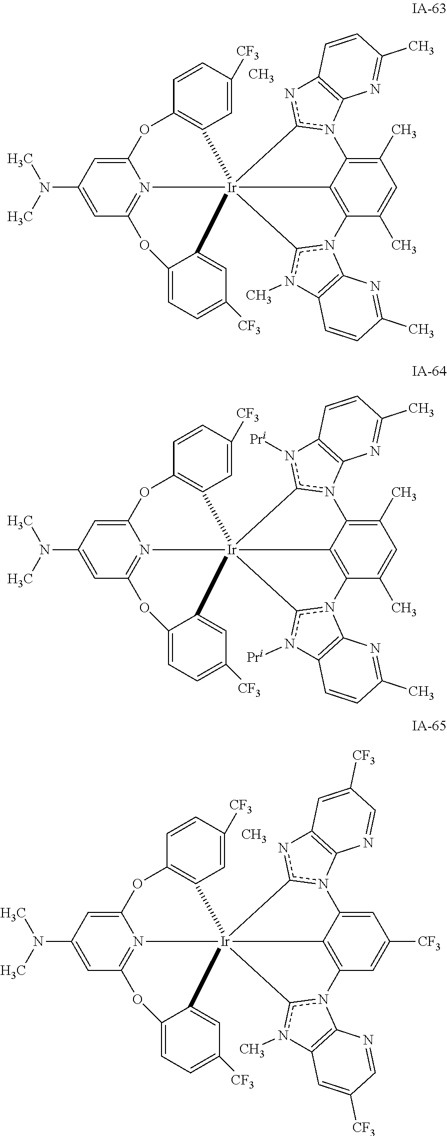

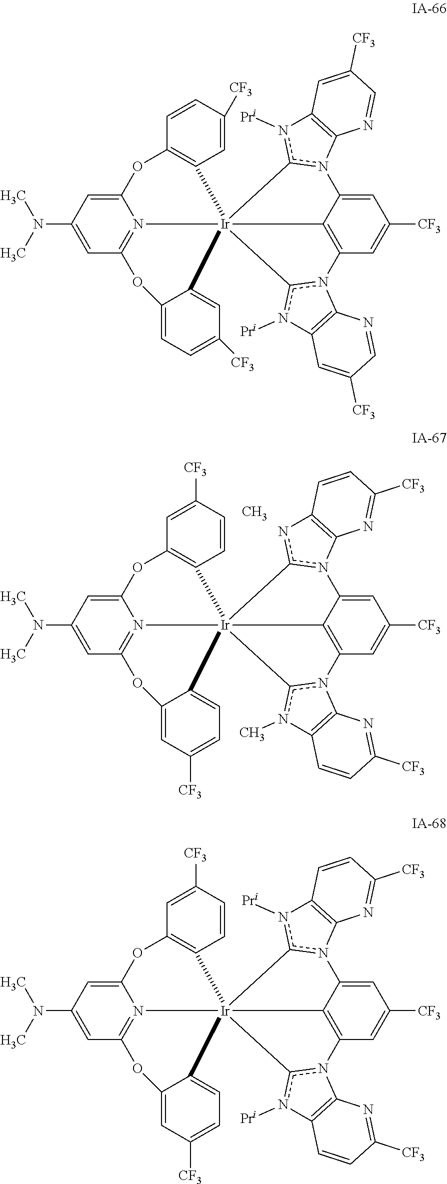

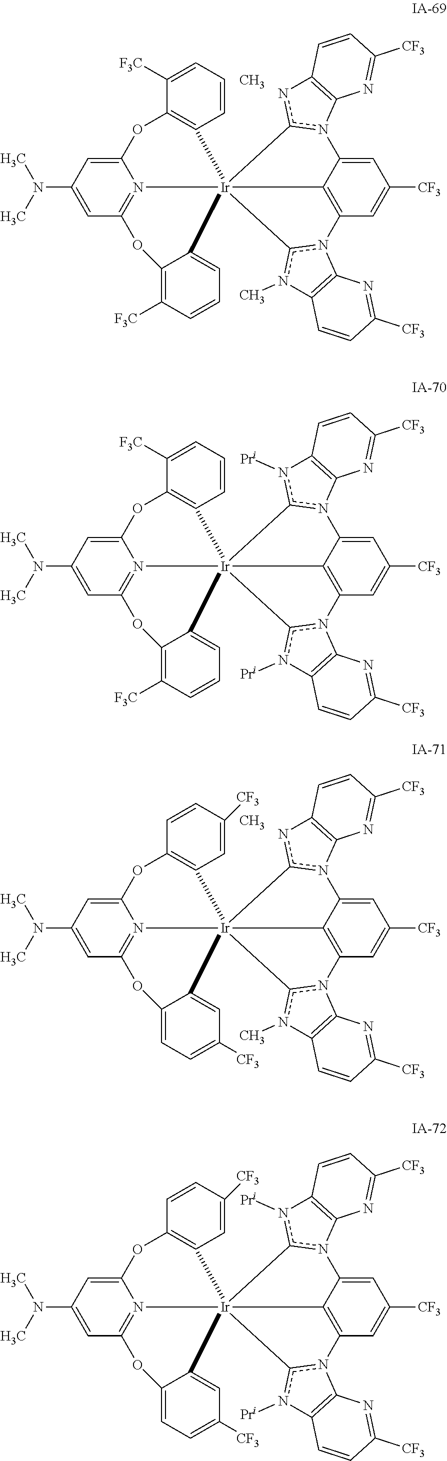

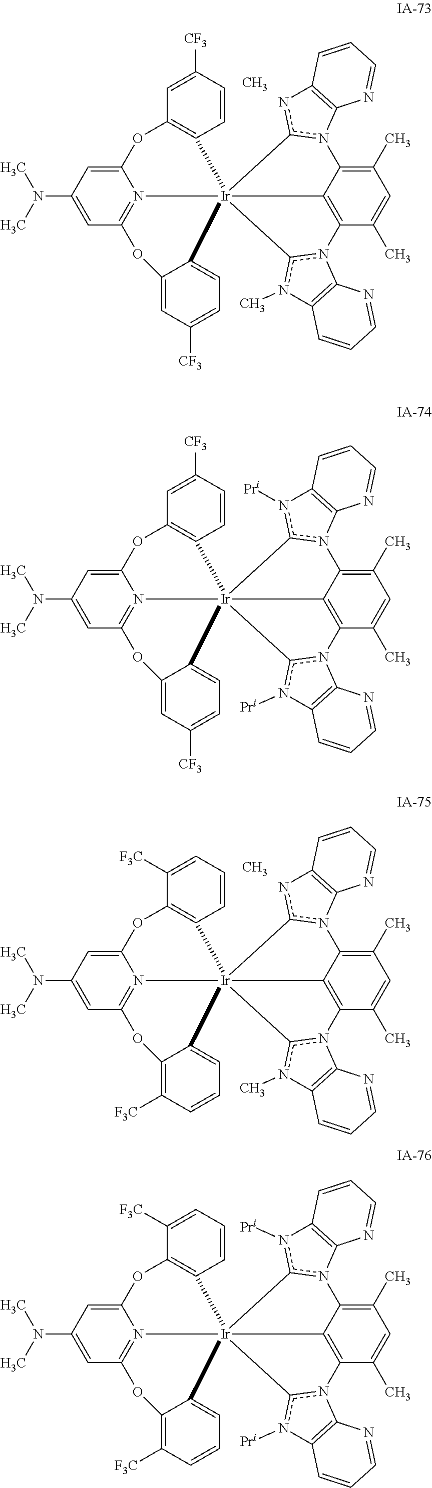

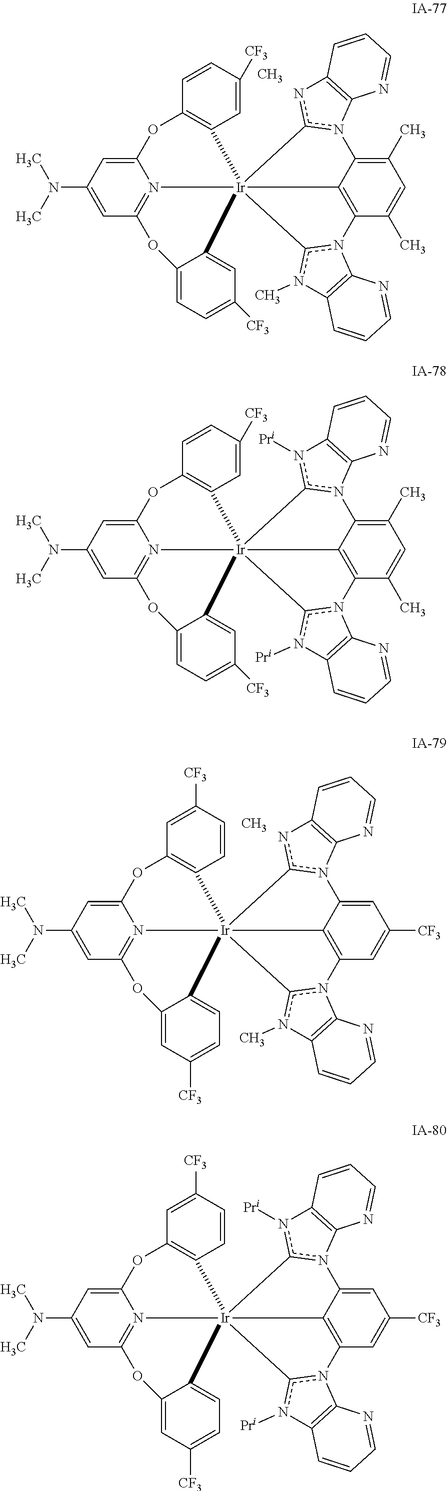

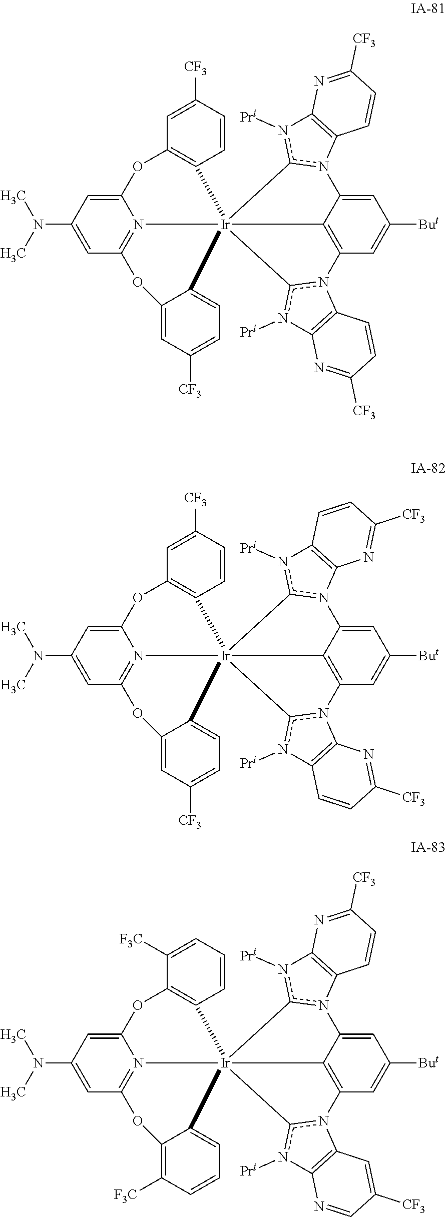

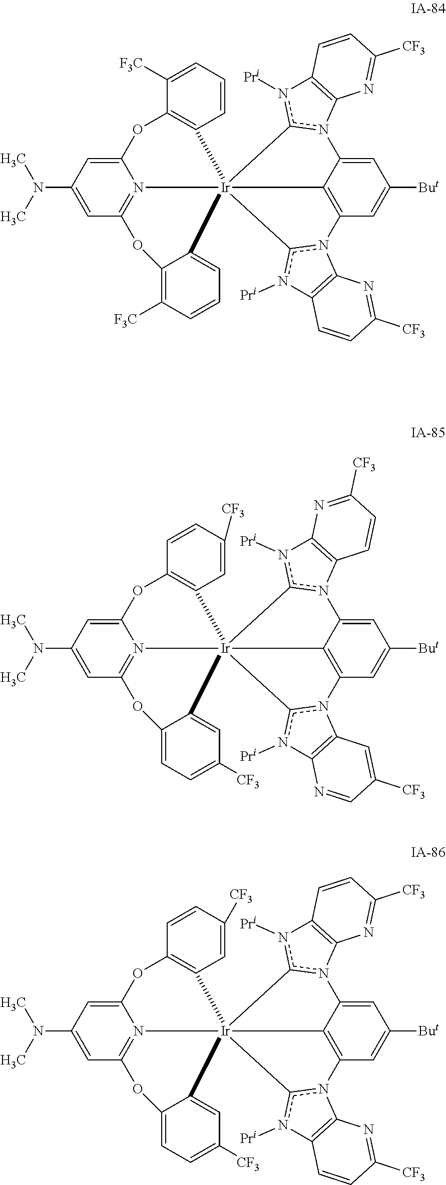

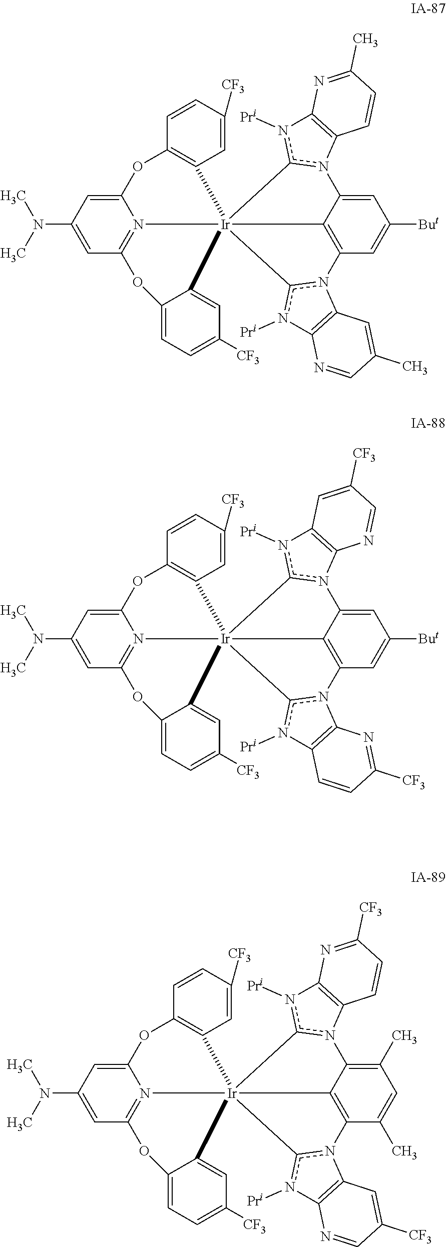

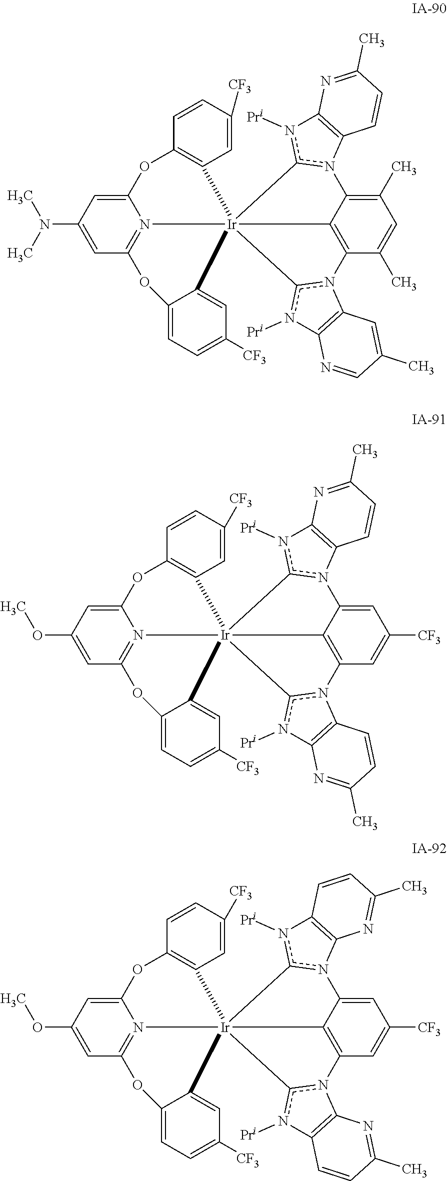

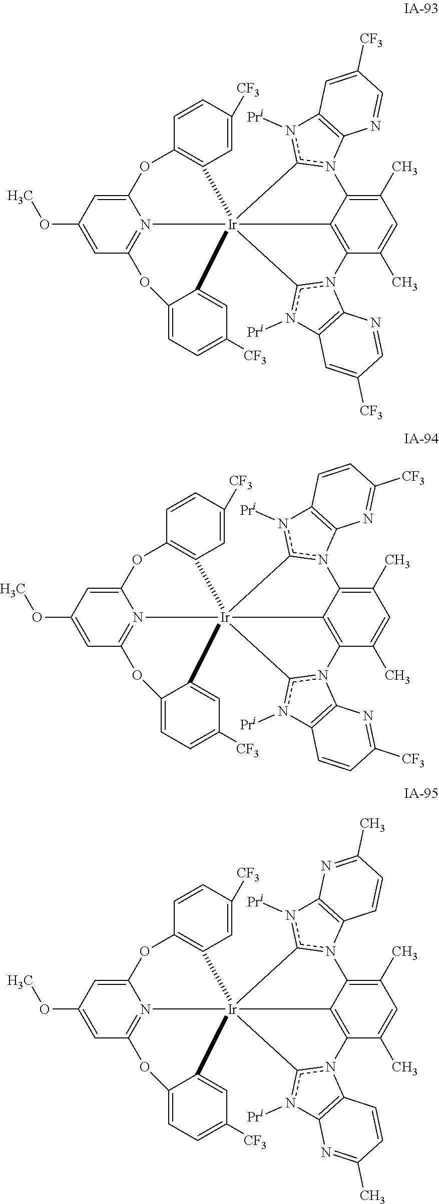

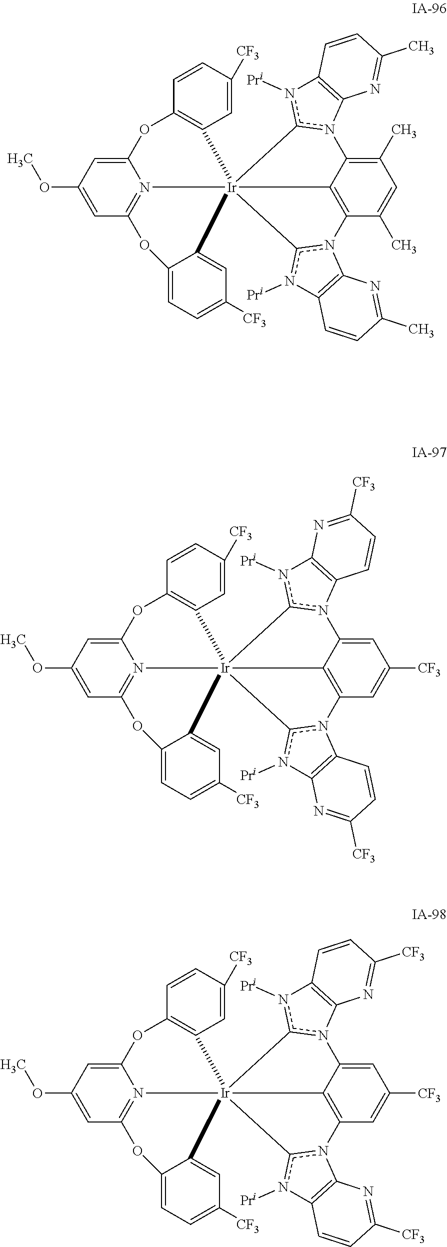

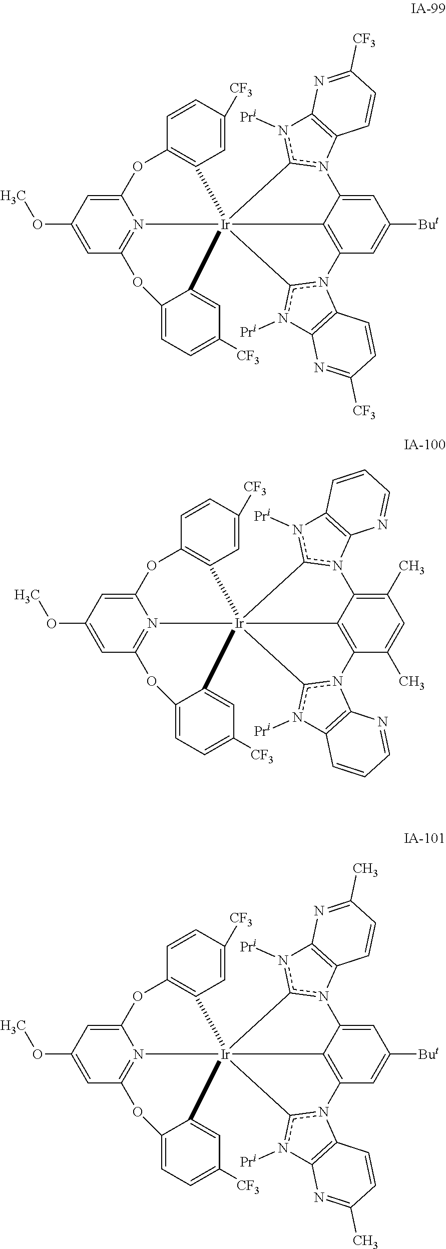

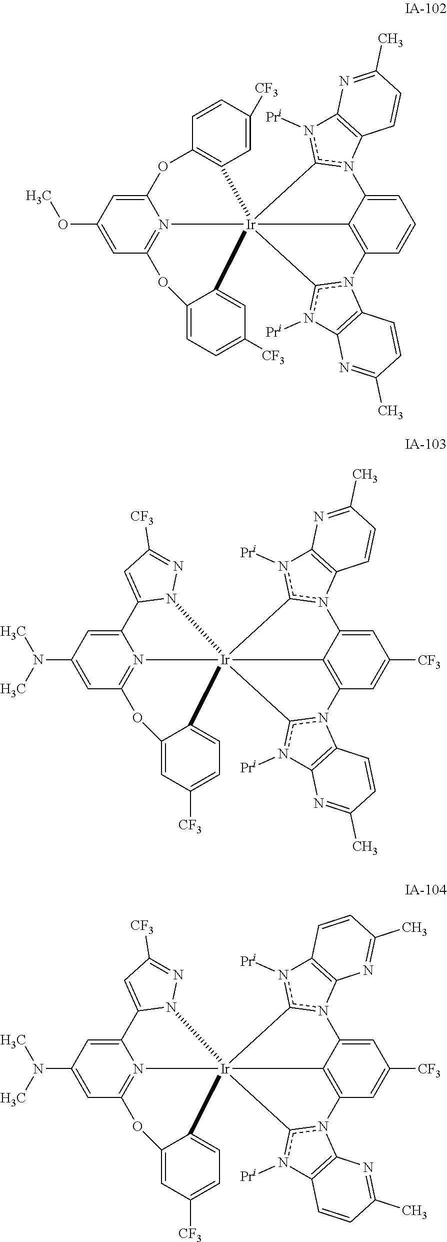

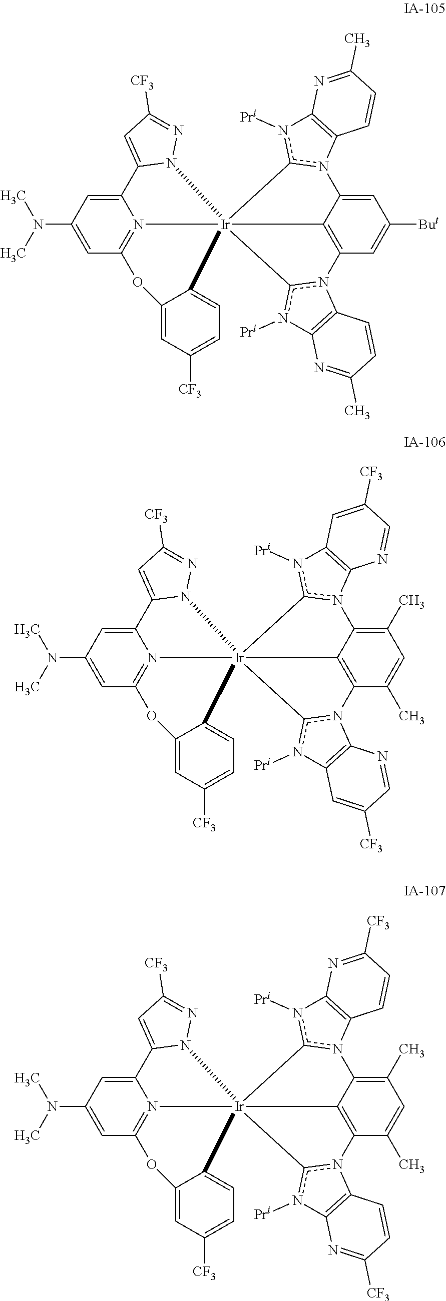

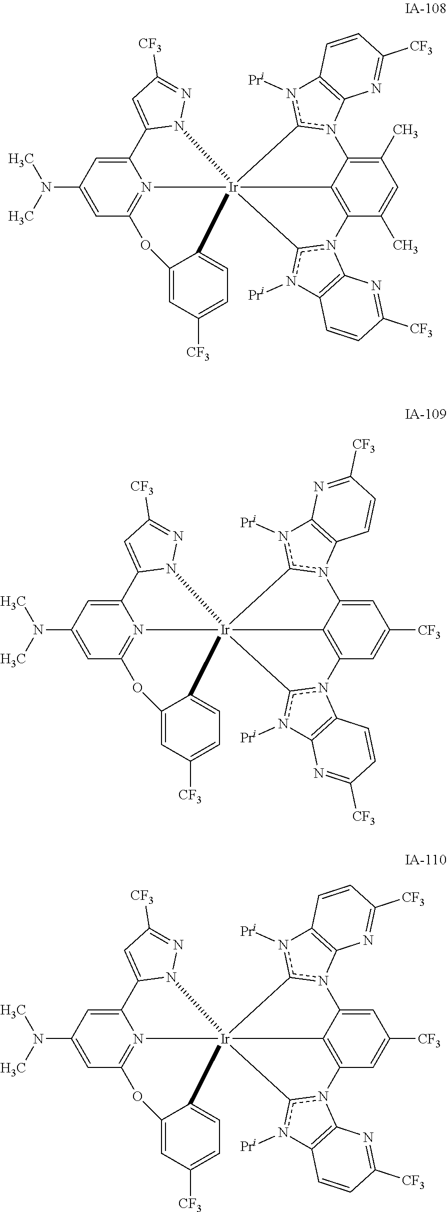

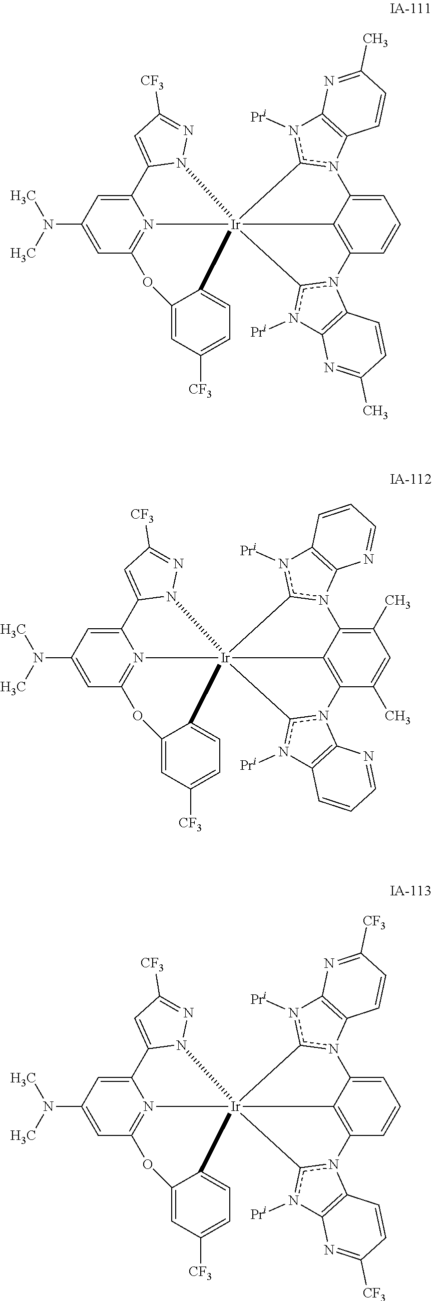

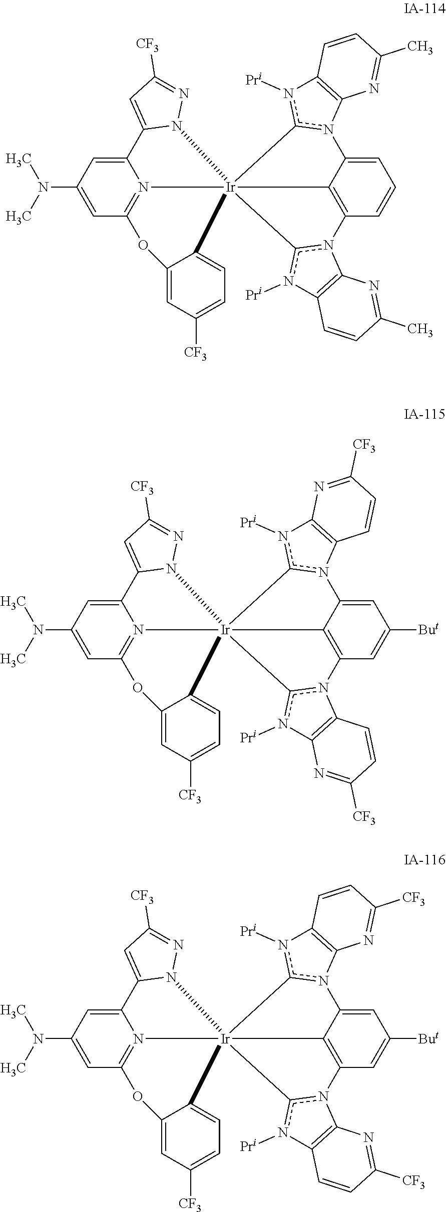

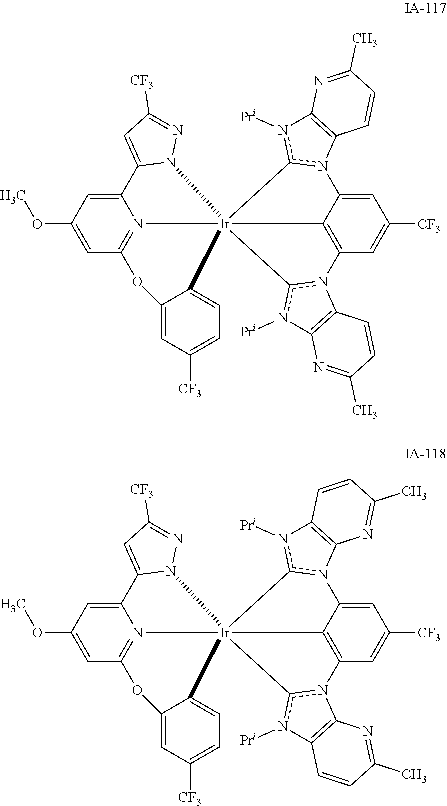

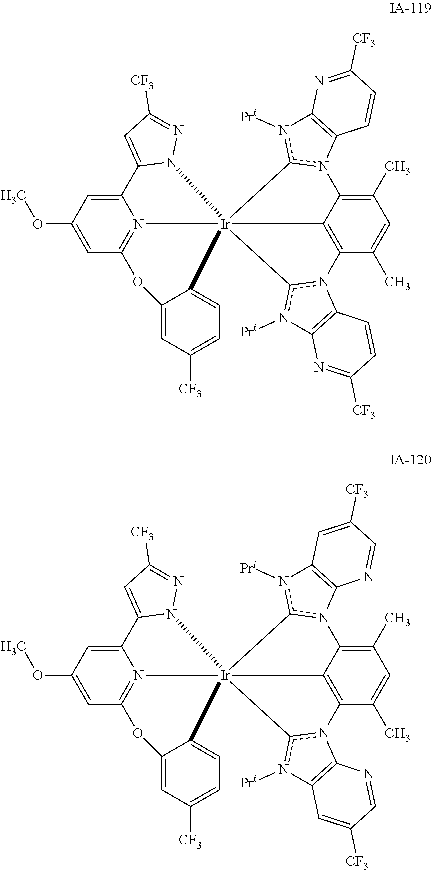

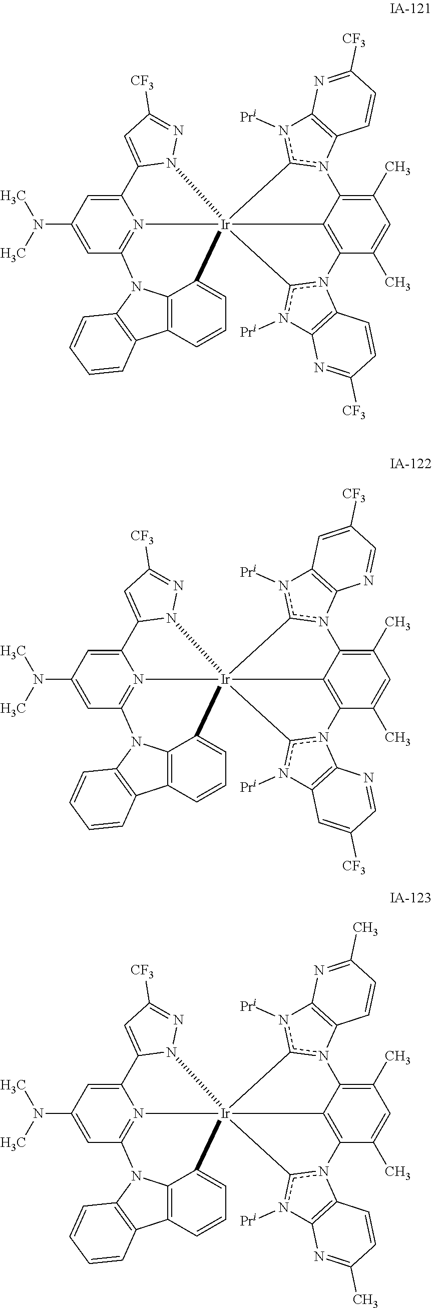

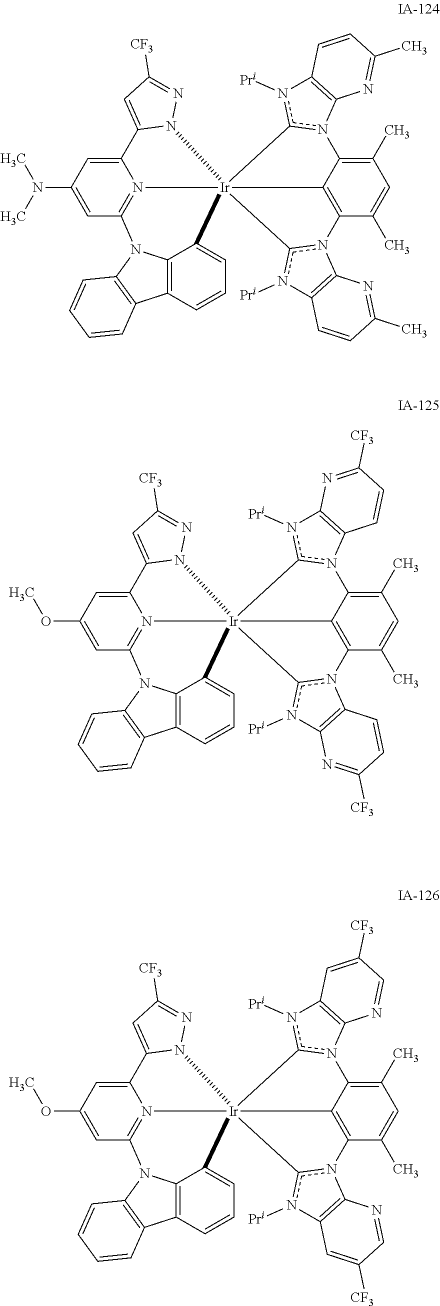

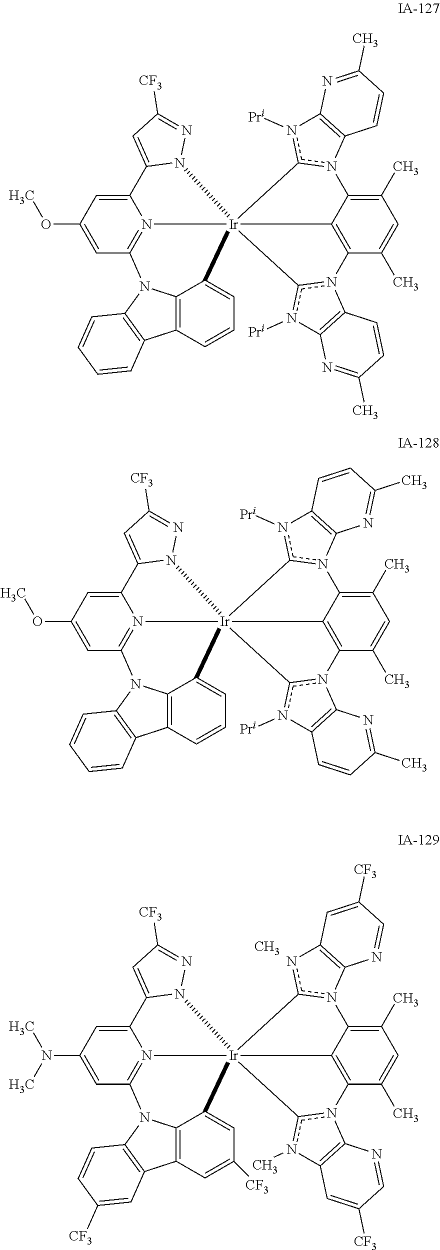

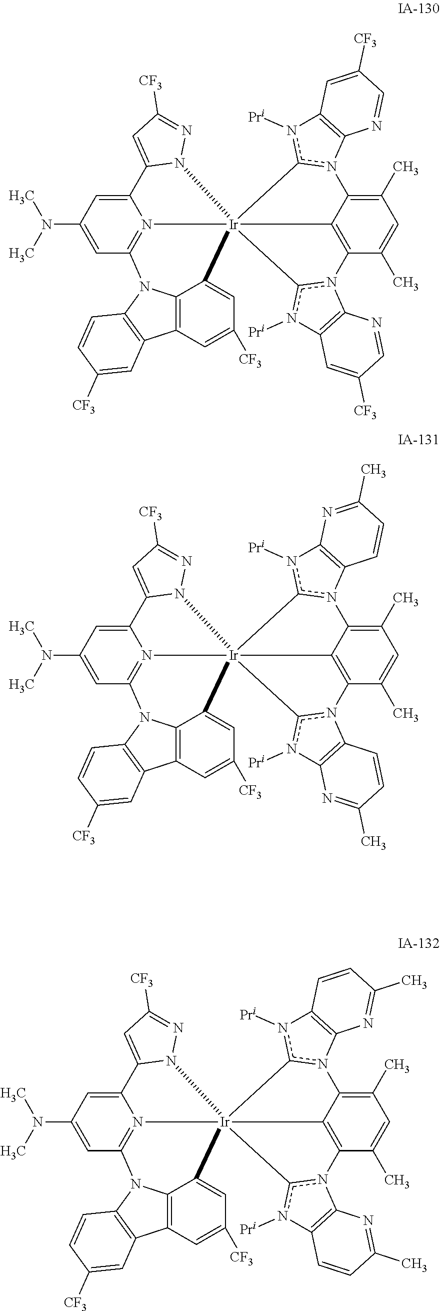

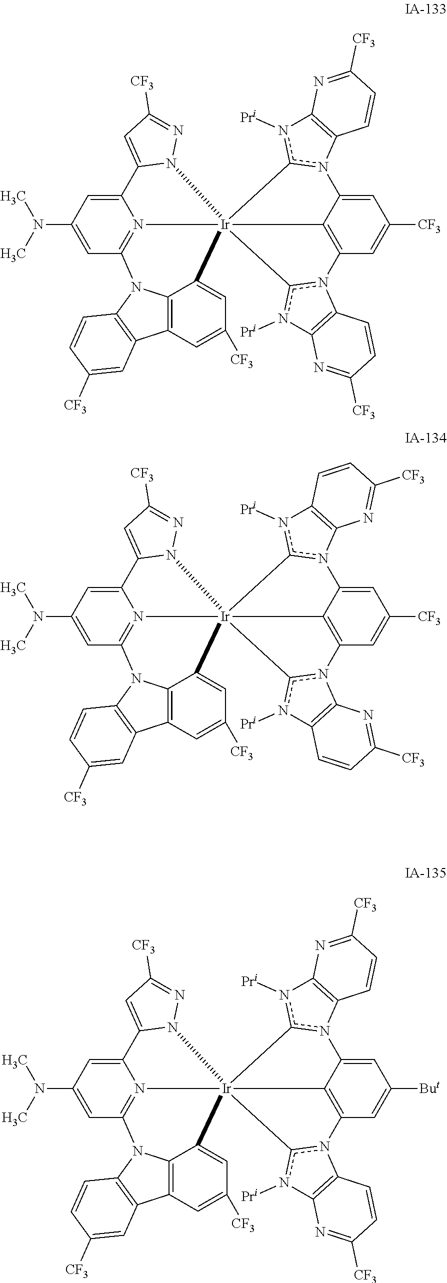

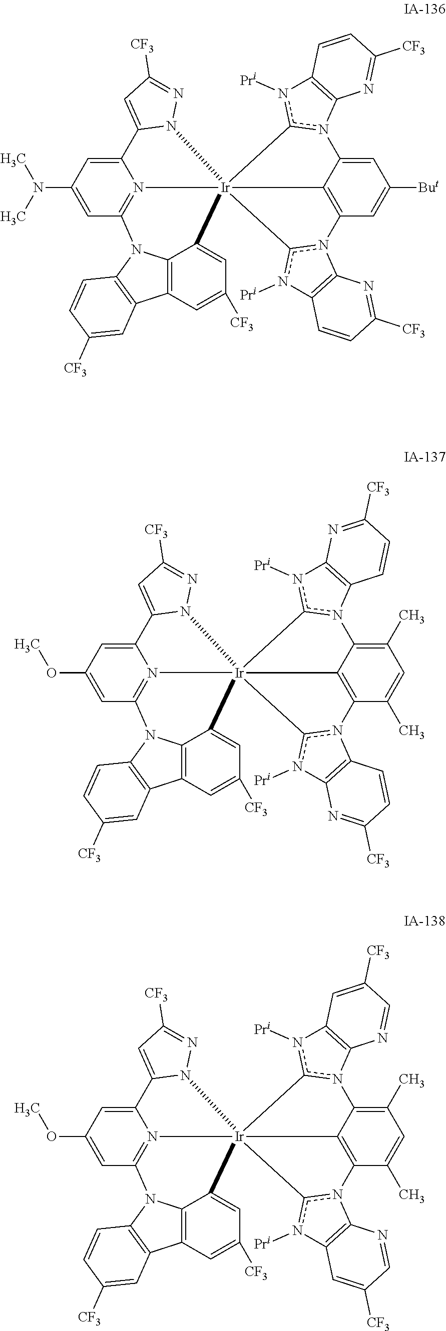

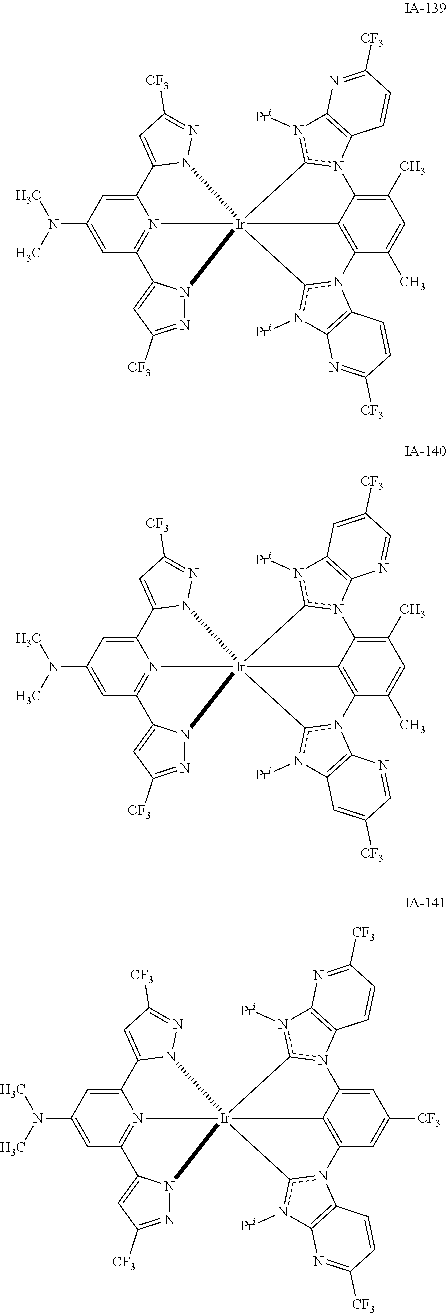

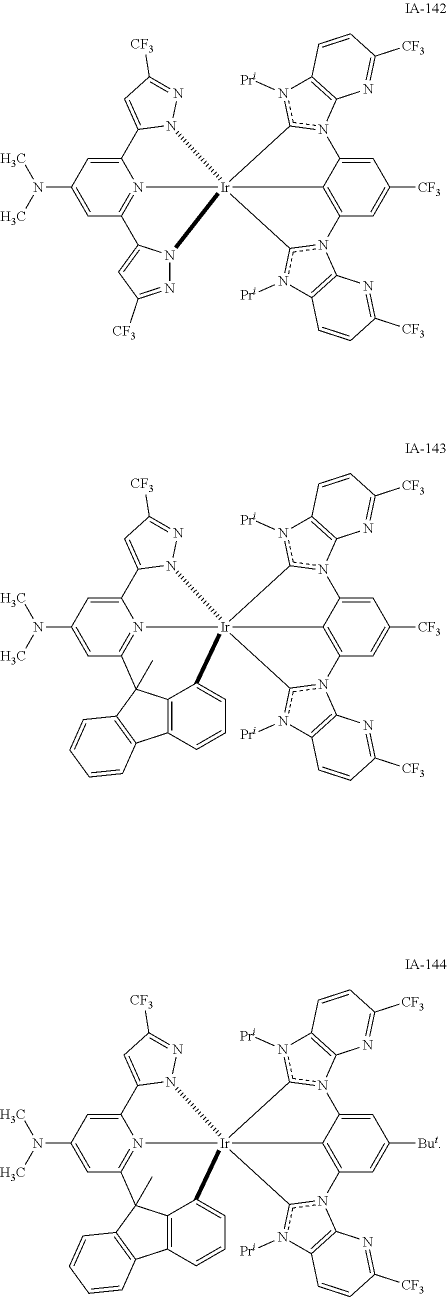

4. The iridium complex of claim 1, wherein a light-emitting peak wavelength of the iridium complex is between 430 nm and 550 nm.

5. An organic light-emitting diode, comprising two electrodes and a light-emitting layer disposed between the two electrodes, wherein the light-emitting layer comprises the iridium complex of claim 1.

6. An iridium complex, having a structure represented by general formula (I-1) or (I-2): 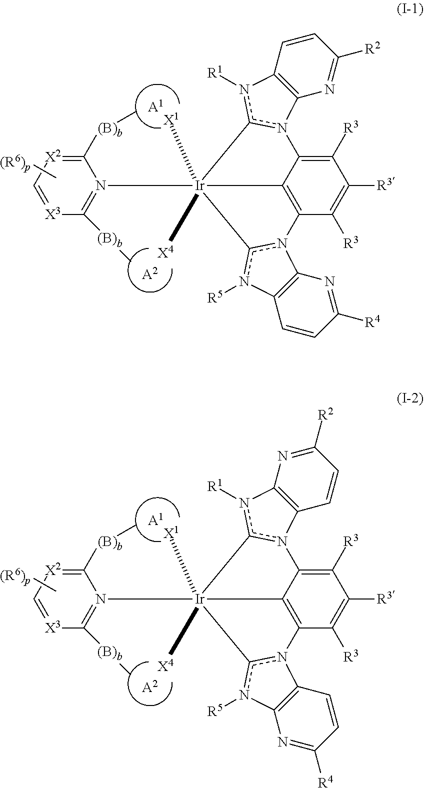
wherein
X¹, X², X³, and X⁴ are each independently carbon or nitrogen;
R¹ and R⁵ are each independently substituted or unsubstituted C₁-C₁₂ alkyl;
R², R³, and R⁴ are each independently hydrogen, substituted or unsubstituted C₁-C₁₂ alkyl, or substituted or unsubstituted C₆-C₁₂ aryl;
R^{3′} is an electron withdrawing group or an electron donating group, wherein the electron withdrawing group comprises —CₘF₂ₘ₊₁, m is an integer of 0 to 3, and the electron donating group comprises methyl, tert-butyl, or C₁-C₆ alkoxy;
A¹ and A² are each independently an unsaturated 5-membered ring or an unsaturated 6-membered ring;
B is —O—, —NR—, or —CR₂—, R is hydrogen or substituted or unsubstituted C₁-C₁₂ alkyl, and —NR— or —CR₂— may optionally be connected to A² to form a substituted or unsubstituted C₉-C₁₄ aromatic ring or a nitrogen-containing heteroaromatic ring;
b is 0 or 1;
R⁶ is hydrogen, fluorine, substituted or unsubstituted C₁-C₁₂ alkyl, substituted or unsubstituted C₁-C₆ alkoxy, substituted or unsubstituted C₆-C₁₂ aryl, substituted or unsubstituted C₁-C₆ amino, or —CₓF₂ₓ₊₁, and x is an integer of 1 to 3;
p is an integer of 1 to 3;
when p is equal to or greater than 2, two or more R⁶'s may be connected to each other to form a C₃-C₈ aromatic ring or a nitrogen-containing heteroaromatic ring;
in general formula (I-1), at least one of R² and R³ is not hydrogen, and at least one of R³ and R⁴ is not hydrogen; and
in general formula (I-2), R² and R⁴ are both not hydrogen.

7. The iridium complex of claim 6, having a structure represented by general formula (I-1A) or (I-2A): 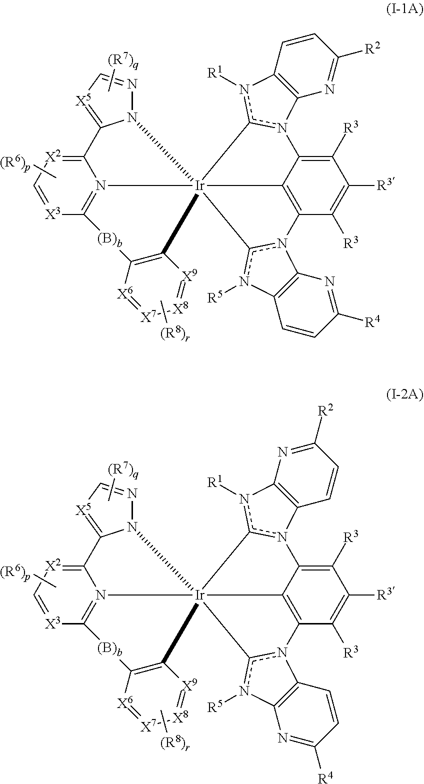
wherein
X⁵, X⁶, X⁷, X⁸, and X⁹ are each independently carbon or nitrogen;
R⁷ and R⁸ are each independently hydrogen, substituted or unsubstituted C₁-C₁₂ alkyl, substituted or unsubstituted C₁-C₆ alkoxy, substituted or unsubstituted C₆-C₁₂ aryl, or —CₓF₂ₓ₊₁, and x is an integer of 1 to 3;
q is an integer of 1 to 2;
r is an integer of 1 to 4;
when q is equal to or greater than 2, two or more R⁶'s may be connected to each other to form a C₃-C₈ aromatic ring or a nitrogen-containing heteroaromatic ring; and
when r is equal to or greater than 2, two or more R⁷'s may be connected to each other to form a C₃-C₈ aromatic ring or a nitrogen-containing heteroaromatic ring.

8. A nitrogen-containing tridentate carbene chelate, having a structure represented by general formula (II): 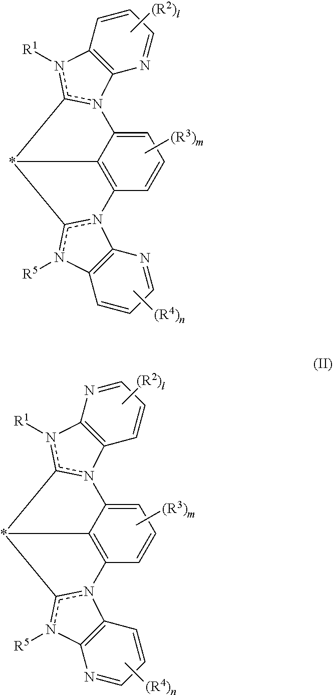
wherein
* represents a bonding site;
one of X′ and X″ is nitrogen, and the other is carbon;
R¹ and R⁵ are each independently substituted or unsubstituted C₁-C₁₂ alkyl;
R², R³, and R⁴ are each interpedently hydrogen, substituted or unsubstituted C₁-C₁₂ alkyl, substituted or unsubstituted C₆-C₁₂ aryl, or —CₘF₂ₘ₊₁, and m is an integer of 0 to 3;
l, m, and n are each independently an integer of 1 to 3;
when l is equal to or greater than 2, two or more R²'s may be connected to each other to form a C₃-C₈ aromatic ring or a nitrogen-containing heteroaromatic ring;
when m is equal to or greater than 2, two or more R³'s may be connected to each other to form a C₃-C₈ aromatic ring or a nitrogen-containing heteroaromatic ring; and
when n is equal to or greater than 2, two or more R⁴'s may be connected to each other to form a C₃-C₈ aromatic ring or a nitrogen-containing heteroaromatic ring.

9. The nitrogen-containing tridentate chelate of claim 8, having a structure represented by general formula (II-1) or (II-2): 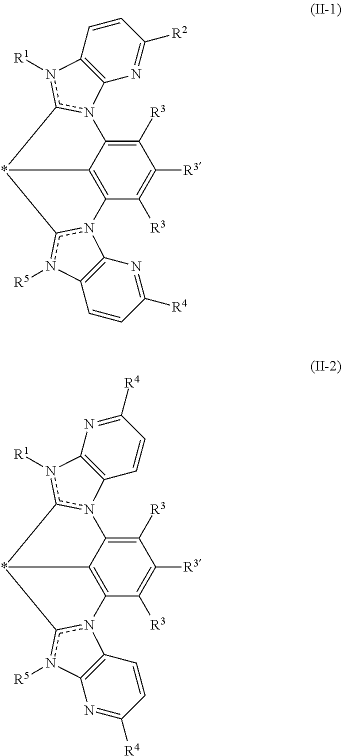
wherein
R², R³, and R⁴ are each independently hydrogen, substituted or unsubstituted C₁-C₁₂ alkyl, or substituted or unsubstituted C₆-C₁₂ aryl;
R^{3′} is an electron withdrawing group or an electron donating group, the electron withdrawing group comprises —CₘF₂ₘ₊₁, m is an integer of 0 to 3, and the electron donating group comprises methyl, tert-butyl, or C₁-C₆ alkoxy;
in general formula (II-1), at least one of R² and R³ is not hydrogen, and at least one of R³ and R⁴ is not hydrogen; and
in general formula (II-2), R² and R⁴ are both not hydrogen.
